(19) United States
(12) Patent Application Publication
(10) Pub. No.: US 2012/0022510 A1
(43) Pub. Date: Jan. 26, 2012

(54) THERMAL SURGERY SAFETY APPARATUS AND METHOD

(75) Inventors: Richard Shaun Welches, Manchester, NH (US); James Henry Boll, Newton, MA (US); Mirko Mirkov, Chelmsford, MA (US); Rafael Armando Sierra, Palmer, MA (US)

(73) Assignee: CYNOSURE, INC., Westford, MA (US)

(21) Appl. No.: 13/254,478

(22) PCT Filed: Mar. 5, 2010
(86) PCT No.: PCT/US10/26415

§ 371 Date: Oct. 6, 2011

Related U.S. Application Data
(60) Provisional application No. 61/157,862, filed on Mar. 5, 2009.

(51) Int. Cl. A61B 18/24; A61B 18/20; A61B 18/22; A61B 18/18

(52) U.S. Cl. 606/3; 606/14; 606/15

(57) ABSTRACT

A laser surgical method is disclosed including: providing a laser surgical device including a handpiece including: an optical delivery component that transmits laser energy from a source to a treatment volume; and an accelerometer configured to provide information indicative of the position of the handpiece. The method includes using the handpiece to transmit laser energy from the source to a plurality of positions within the treatment volume; using the accelerometer, providing information indicative of the position of the handpiece; determining information indicative of an amount of energy delivered at each of the plurality of positions within the treatment volume based on the information indicative of the position of the handpiece, and displaying a graphical representation indicative of the amount of energy delivered at each of the plurality of positions within the treatment volume.

### CROSS REFERENCE TO RELATED APPLICATIONS

The present application claims benefit of U.S. Provisional Patent Application Ser. No. 61/157,862 filed Mar. 5, 2009, the entire contents of which is incorporated by reference herein in its entirety.

The present application claims benefit of U.S. Provisional Application Ser. No. 60/987,596, filed Nov. 13, 2007, U.S. Provisional Application Ser. No. 60/987,617, filed Nov. 13, 2007, U.S. Provisional Application Ser. No. 60/987,819, filed Nov. 14, 2007, U.S. Provisional Application Ser. No. 60/987,821, filed Nov. 14, 2007, U.S. Provisional Application Ser. No. 61/018,727, filed Jan. 3, 2008, U.S. Provisional Application Ser. No. 61/018,729, filed Jan. 3, 2008, and U.S. Provisional Application Ser. No. 60/933,736, filed Jun. 8, 2007, the contents of each of which are incorporated by reference herein in their entirety

### BACKGROUND

To improve one's health or shape, patients have resorted to surgical methods for removing undesirable tissue from areas of their body. For example, to remove fat tissue, some patients have preferred liposuction, a procedure in which fat is removed by suction mechanism because despite strenuous dieting and exercise, some of the patients cannot lose fat, particularly in certain areas. Alternatively, laser or other light sources has been applied for heating, removal, destruction (for example, killing), photocoagulation, eradication or otherwise treating (hereinafter collectively referred as “treating” or “treatment”) the tissue.

Because the treatment mechanism are implemented beneath the skin of the patient, a clinician cannot assess the extent of the treatment or the condition of the treated portions of the treatment area by, for example, a type of visual aid. As such, the clinician has no other means to determine the extent of the treatment or to guide the instrument(s) to the untreated portions of the treatment area except by the means of feel. In turn, it is not uncommon during the procedure to result uneven removal of the undesired tissue which may leave an esthetically unattractive patterning on the patient's skin.

Further, in typical applications, there is no direct method to ascertain the tissue type in front of the laser delivery fiber during procedures such as laser lipolysis. The physician relies on his knowledge of anatomy and human physiology to position the fiber tip in the unwanted fat layer. The physician is aided by a visible aiming beam carrying a single or multitude of wavelengths through the delivery fiber. A skillful physician can correlate the aiming beam visibility with the fiber tip position and depth under the skin. However, even for a skillful physician is very hard (nearly impossible) to determine the type of tissue in front of the fiber tip.

Furthermore, while the tissue can be treated using laser or light energy source as a result of absorption in the tissue of the energy source, the surgical instruments lack a mechanism that accounts the amount of power absorbed by the treated portions of the treatment area. As such, the clinician can under-treat or over-treat, resulting an incomplete removal of the tissue or charring thereof due to overexposure.

### SUMMARY OF THE INVENTION

The inventors have realized that by providing one or more sensors for use in a medical environment where energy in directed to target tissue (e.g. laser surgical procedure), increased safety and ease of use may be obtained. By combining different types of sensor inputs, a wealth of information can be provided characterizing an ongoing medical procedure.

For example, the inventors describe herein methods and devices that include mechanisms to detect the motion of a surgical device used during a procedure for removing undesired tissue or body parts.

Application of power into tissue results in a local temperature rise according to absorbance of constituent tissues. Propagation distance is dependent to, for example, wavelength/tissue type. Further, each tissue type has an associated time constant and thermal conductivity. Thus, in principle, tissue temperature rise in vivo can be determined from knowledge of the constituent tissues, the wavelength and power directed thereto as long as the position of the energy delivery component of the device, which is inserted into the treatment area is known.

According to one aspect of the present invention, the position of the energy delivery component can be determined by processing the acceleration of the device, which is integrated to provide a speed feedback. Accounting the speed feedback, the device can control the amount of the power directed to a treatment area in relation to the value of the speed feedback. For example, the device can stop emitting the energy directed to the treatment area when the device is not moving or moving at a speed below a predetermined value to prevent excessive in vivo thermal effect. The speed feedback may also be used to control the applied dose of energy, e.g. to maintain a fixed energy deposited in the tissue per unit traveled.

According to another aspect of the present invention, the position of the energy delivery component can be determined by taking the first integration of speed (or the 2″ integration of acceleration) to provide a position feedback of the energy delivery component within the treatment area. Power controlling for the position feedback application is done with a power vs. difference-in-position algorithm. For example, each energy discharge/shot into tissue in the treatment area is assigned a 3-D Cartesian point on an 8 quadrant place. Each point on the Cartesian reference place represents a “heat container”. The heat containers accumulate the bleed off counts according to energy applied or energy-in (Eᵢₙ), absorbance vs. propagation distance, baseline temperature, and the time constant and conductivity associated with the tissue type. Additional sensor inputs such as tissue type measurement and or direct or indirect temperature measurement can be used in conjunction with the positional information to augment or confirm the spatial energy distribution information.

In one aspect, a laser surgical apparatus is disclosed including: a handpiece including an optical delivery component that transmits laser energy from a source to a treatment volume; and an accelerometer configured to provide information indicative of the position of the handpiece. The apparatus includes a processor coupled to the accelerometer and the source and controlling the laser energy transmitted to the treatment volume; and a display. The processor is configured to determine information indicative of an amount of energy delivered at each of a plurality of positions within the treatment volume based on the information indicative of the position of the handpiece. The display is configured to display a graphical representation indicative of the amount of energy delivered at each of the plurality of positions within the treatment volume.

In some embodiments, the processor is configured to control the amount of energy delivered to the treatment volume based on feedback from the accelerometer.

In some embodiments, the accelerometer measures acceleration along three axes.

In some embodiments, the accelerometer is a gyro compensated accelerometer.

In some embodiments, the graphical representation includes a map of the treatment volume, where a plurality of points on the map correspond to the plurality of positions within the treatment volume, and where the a graphical quality of each of the points depends on the amount of energy delivered at the position within the treatment volume.

In some embodiments, the graphical representation is a three dimensional representation.

In some embodiments, the handpiece further includes a temperature sensor configured to provide information indicative of the temperature of tissue at positions within the treatment volume. The processor is coupled to the temperature sensor and is configured to determine information indicative of the temperature of each of a plurality of positions within the treatment volume based on the information indicative of the position of the handpiece and the information indicative of the temperature of tissue at positions within the treatment volume. The display is configured to display a graphical representation indicative of the amount of energy delivered at each of the plurality of positions within the treatment volume.

In one aspect, a laser surgical method is disclosed including: providing a laser surgical device including a handpiece including: an optical delivery component that transmits laser energy from a source to a treatment volume; and an accelerometer configured to provide information indicative of the position of the handpiece. The method includes using the handpiece to transmit laser energy from the source to a plurality of positions within the treatment volume; using the accelerometer, providing information indicative of the position of the handpiece; determining information indicative of an amount of energy delivered at each of the plurality of positions within the treatment volume based on the information indicative of the position of the handpiece, and displaying a graphical representation indicative of the amount of energy delivered at each of the plurality of positions within the treatment volume.

Some embodiments include including controlling the amount of energy delivered to the plurality of positions within the treatment volume based on feedback from the accelerometer.

In some embodiments, accelerometer measures acceleration along three axes.

In some embodiments, the accelerometer is a gyro compensated accelerometer.

In some embodiments, the graphical representation includes a map of the treatment volume, where a plurality of points on the map correspond to the plurality of positions within the treatment volume, and where the a graphical quality of each of the points depends on the amount of energy delivered at the position within the treatment volume.

In some embodiments, the graphical representation is a three dimensional representation.

In some embodiments, the handpiece further includes a temperature sensor configured to provide information indicative of the temperature of tissue at positions within the treatment volume, and the processor is coupled to the temperature sensor. Such embodiments include using the temperature sensor, determining information indicative of the temperature of each of a plurality of positions within the treatment volume based on the information indicative of the position of the handpiece and the information indicative of the temperature of tissue at positions within the treatment volume, and displaying a graphical representation indicative of the amount of energy delivered at each of the plurality of positions within the treatment volume.

In another aspect, a laser surgical apparatus is disclosed including: a handpiece including: an optical delivery component that transmits laser energy from a source to a treatment volume; and an accelerometer configured to provide information indicative of acceleration of the handpiece along three axes. The apparatus includes a processor coupled to the accelerometer and the source and controlling the laser energy transmitted to the treatment volume based on feedback from the accelerometer.

Some embodiments include a gyroscope configured to provide information indicative of the spatial orientation of the handpiece, and where the processor is coupled to the gyroscope and is configured to control the laser energy transmitted to the treatment volume based on feedback from the accelerometer and the gyroscope.

In some embodiments, the processor is configured to determine information indicative of an absolute position of the handpiece based on the information indicative of acceleration of the handpiece along three axes, and the information indicative of the spatial orientation of the handpiece.

In some embodiments, the processor is configured to determine information indicative of a speed of the handpiece based on the information indicative of acceleration of the handpiece along three axes; and control the laser energy transmitted to the treatment volume based on feedback using the information indicative of the speed of the handpiece.

In some embodiments, the information indicative of acceleration of the handpiece along three axes includes, for at least one axis, a signal having an amplitude which depends on the acceleration of the handpiece along the axis.

In some embodiments, the processor is configured to selectively block low frequency components of the signal prior to integrating the signal to determine information indicative of a speed of the handpiece along the respective axis. In some embodiments, the processor is configured to determine the speed of the handpiece along each of the three axes based one information indicative of acceleration of the handpiece along three axes; determine a weighted average speed of the handpiece by calculating a weighted average of the speeds of the handpiece along each of the three axes; and control the laser energy transmitted to the treatment volume based on feedback using the weighted average speed of the handpiece.

In some embodiments, the handpiece includes a probe member for insertion into the treatment volume, the probe member extending along a probe member axis, the accelerometer is configured to provide information indicative of acceleration along each of the three axes, one of the three axes being substantially parallel to the probe member axis; and the processor is configured to determined the weighted average speed of the handpiece by calculating a weighted average of the speeds of the handpiece along each of the three axes, where the axis substantially parallel to the probe member axis is given greater weight that the other axes.

In another aspect, a laser surgical method is disclosed including: providing a handpiece including: an optical delivery component that transmits laser energy from a source to a treatment volume; and an accelerometer configured to provide information indicative of acceleration of the handpiece along three axes; using the handpiece to transmit laser energy from the source to the treatment volume; using the accelerometer, providing information indicative of acceleration of the handpiece along three axes; and controlling the laser energy transmitted to the treatment volume based on feedback from the accelerometer.

In some embodiments, the handpiece further includes a gyroscope, and the method includes using the gyroscope, providing information indicative of the spatial orientation of the handpiece, and further including; and controlling the laser energy transmitted to the treatment volume based on feedback from the accelerometer and the gyroscope.

Some embodiments include: determining information indicative of an absolute position of the handpiece based on the information indicative of acceleration of the handpiece along three axes, and the information indicative of the spatial orientation of the handpiece.

Some embodiments include: determining information indicative of a speed of the handpiece based on the information indicative of acceleration of the handpiece along three axes; and controlling the laser energy transmitted to the treatment volume based on feedback using the information indicative of the speed of the handpiece.

Some embodiments include determining the speed of the handpiece along each of the three axes based one information indicative of acceleration of the handpiece along three axes; determining a weighted average speed of the handpiece by calculating a weighted average of the speeds of the handpiece along each of the three axes; and controlling the laser energy transmitted to the treatment volume based on feedback using the weighted average speed of the handpiece.

In some embodiments, the handpiece includes a probe member extending along a probe member axis. The method further includes:

inserting the probe member into the treatment volume; repetitively advancing and withdrawing the probe member within the treatment volume; using the accelerometer to provide information indicative of acceleration along each of the three axes, one of the three axes being substantially parallel to the probe member axis; and determining the weighted average speed of the handpiece by calculating a weighted average of the speeds of the handpiece along each of the three axes, where the axis substantially parallel to the probe member axis is given greater weight that the other axes.

In another aspect, a laser surgical apparatus is disclosed including: a handpiece including: a probe member including an optical delivery component that transmits laser energy from a source to a treatment volume, the probe member adapted for insertion into a treatment volume through an incision in a patient; and an accelerometer configured to provide information indicative of the position of the handpiece relative to the incision; a processor coupled to the accelerometer and the source and controlling the laser energy transmitted to the treatment volume based on the information indicative of the position of the handpiece relative to the incision.

In some embodiments, the accelerometer is configured to provide information indicative of a speed of the handpiece and the processor is configured to controlling the laser energy transmitted to the treatment volume based on the information indicative of the speed of the handpiece.

In another aspect, a method is disclosed including providing a handpiece including: a probe member including an optical delivery component that transmits laser energy from a source to a treatment volume, the probe member adapted for insertion into a treatment volume through an incision in a patient; and an accelerometer configured to provide information indicative of the position of the handpiece relative to the incision. The method includes inserting the probe member into the treatment volume through the incision; repetitively advancing and withdrawing the probe member within the treatment volume; transmitting laser energy to the treatment volume; using the accelerometer to provide information indicative of the position of the handpiece relative to the incision; and controlling the laser energy transmitted to the treatment volume based on the information indicative of the position of the handpiece relative to the incision.

Some embodiments include: using the accelerometer to provide information indicative of a speed of the handpiece; and controlling the laser energy transmitted to the treatment volume based on the information indicative of the speed of the handpiece.

In another aspect, a laser surgical apparatus is disclosed including: a handpiece including: an optical delivery component that transmits laser energy from a source to a treatment volume; an accelerometer configured to provide acceleration information indicative of an acceleration of the handpiece; and a temperature sensor configured to provide temperature information indicative of a temperature of tissue within the treatment volume. The apparatus includes a processor coupled to the accelerometer, the temperature sensor, and the source and configured to control the laser energy transmitted to the treatment volume based on the acceleration information and the temperature information.

In some embodiments, the handpiece includes a probe member adapted for insertion into the treatment volume through an incision in a patient, the probe member including at least a portion of the optical delivery component.

In some embodiments, the processor is configured to determine speed information indicative of the speed of the handpiece based on the acceleration information; and control the laser energy transmitted to the treatment volume based on the speed information and the temperature information.

In some embodiments, the processor is configured to determine position information indicative of the position of the handpiece based on the acceleration information; and control the laser energy transmitted to the treatment volume based on the position information and the temperature information.

In some embodiments, e the temperature sensor includes at least one selected from the group consisting of: a thermocouple and a thermister.

In some embodiments, the temperature sensor includes an infrared sensor. In some embodiments, the handpiece includes a optical sensing element configured to transmit infrared light from the treatment volume to the infrared sensor.

In some embodiments, the processor is configured to compare the speed of the handpiece to a threshold value, and inhibit the transmittal of laser energy to the treatment volume when the speed is below the threshold value.

In some embodiments, the temperature sensor is configured to measure the temperature of the tissue when the processor inhibits the transmittal of laser energy to the treatment volume or when the processor determines that the speed of the handpiece is below a measurement threshold speed.

In some embodiments, the processor is configured to compare the temperature of the tissue to a threshold value, and inhibit the transmittal of laser energy to the treatment volume when the temperature is above a threshold value.

In some embodiments, the processor is configured to repetitively, at a first repetition rate, compare the speed of the handpiece to a speed threshold value, and inhibit the transmittal of laser energy to the treatment volume when the speed is below the speed threshold value; and repetitively, at a second repetition rate, compare the temperature of the tissue to a temperature threshold value, and inhibit the transmittal of laser energy to the treatment volume when the temperature is above the temperature threshold value.

In some embodiments, the first repetition rate is greater than the second repetition rate.

In some embodiments, the processor is configured to determine information indicative of the temperature of tissue at each of a plurality of positions within the treatment volume.

In some embodiments, processor is configured to control the laser energy transmitted to the treatment volume based on information indicative of the temperature of tissue at each of a plurality of positions within the treatment volume.

Some embodiments including a display configured to show a graphical depiction indicative of the temperature of tissue at each of a plurality of positions within the treatment volume.

In some embodiments, the information indicative of the temperature of tissue at each of a plurality of positions within the treatment volume includes, for each position, a series of temperatures measured at a plurality of times.

In some embodiments, the processor is configured to, for each of the positions, calculate a running average of the series of temperatures.

In some embodiments, the display is configured to display, in real time, a graphical representation of the running averages at each of the positions.

In some embodiments, the accelerometer includes a MEMs device.

In some embodiments, the accelerometer measures accelerations along three axes.

In some embodiments, the accelerometer is a gyro compensated accelerometer.

In some embodiments, controlling the laser energy includes controlling at least one selected from the group consisting of: wavelength, pulse rate, pulse duty cycle, intensity, and fluence.

In another aspect, a laser surgical method is disclosed including: providing a handpiece including: an optical delivery component that transmits laser energy from a source to a treatment volume; an accelerometer configured to provide acceleration information indicative of an acceleration of the handpiece; and a temperature sensor configured to provide temperature information indicative of a temperature of tissue within the treatment volume. The method includes transmitting laser energy to the treatment volume; using the accelerometer to provide acceleration information indicative of an acceleration of the handpiece; using the temperature sensor to provide temperature information indicative of a temperature of tissue within the treatment volume; and controlling the laser energy transmitted to the treatment volume based on the acceleration information and the temperature information.

In some embodiments, e the handpiece includes a probe member and the method includes: inserting the probe member through an incision in a patient into the treatment volume; and delivering laser energy to the treatment area from the probe member.

Some embodiments include: determining speed information indicative of the speed of the handpiece based on the acceleration information; and controlling the laser energy transmitted to the treatment volume based on the speed information and the temperature information.

In some embodiments, the processor is configured to determine position information indicative of the position of the handpiece based on the acceleration information; and control the laser energy transmitted to the treatment volume based on the position information and the temperature information.

Some embodiments include: comparing the speed of the handpiece to a threshold value, and inhibiting the transmittal of laser energy to the treatment volume when the speed is below the threshold value.

Some embodiments include: using the temperature sensor to measure the temperature of the tissue when the processor inhibits the transmittal of laser energy to the treatment volume or when the processor determines that the speed of the handpiece is below a measurement threshold speed.

Some embodiments include: comparing the temperature of the tissue to a threshold value, and inhibit the transmittal of laser energy to the treatment volume when the temperature is above a threshold value.

Some embodiments include: determining information indicative of the temperature of tissue at each of a plurality of positions within the treatment volume; and controlling the laser energy transmitted to the treatment volume based on information indicative of the temperature of tissue at each of a plurality of positions within the treatment volume.

Some embodiments include displaying a graphical depiction indicative of the temperature of tissue at each of a plurality of positions within the treatment volume.

In some embodiments, the information indicative of the temperature of tissue at each of a plurality of positions within the treatment volume includes, for each position, a series of temperatures measured at a plurality of times. The method includes, for each of the positions, calculating a running average of the series of temperatures; and displaying, in real time, a graphical representation of the running averages at each of the positions.

In one aspect, a method is disclosed of treating cellulite in a patient. The method includes inserting an optical delivery device into the patient such that a light emitting portion of the device is located below the interface between the dermis and the hypodermis of the patient; and delivering therapeutic light from the light emitting portion of the delivery device to heat a target region located proximal to the interface to cause thermal damage in the target region without causing substantial thermal damage to dermal and epidermal tissue located above the target region. In one embodiment, the step of delivering therapeutic light from the light emitting portion of the delivery device to heat a target region located proximal to the interface comprises substantially localizing the heating of the dermis to within a desired distance above the interface. In some embodiments, the desired distance is about 0.5 mm, 1.0 mm, or less. In one embodiment, the method includes heating the target region proximal the interface to a temperature of about 50° C. or more while maintaining the upper dermal and epidermal tissue located above the target region at a temperature of about 42° C. or less. In another embodiment, the target region includes at least one adipocyte extending through the interface into the dermis, and where the thermal damage includes thermal denaturing of the adipocyte. In yet another embodiment, the target region includes connective tissue which connects the dermis to underlying hypodermal tissue, and where the thermal damage includes damage to the connective tissue. In one embodiment, the method further includes inserting a tip of a cannula into the target region; and moving the tip of the cannula within the target region to cause mechanical damage to tissue in the region. In another embodiment, the target region includes connective tissue which connects the dermis to underlying hypodermal tissue, and where the mechanical damage includes damage to the connective tissue. In one embodiment, the optical delivery device includes an optical fiber having at least a portion housed in the cannula. In another embodiment, the optical delivery device includes a side firing optical fiber which extends along a longitudinal axis from a first end to a second end, and where the step of delivering therapeutic light from the light emitting portion of the delivery device includes: receiving therapeutic light at the first end of the fiber; transmitting the therapeutic light to the second end of the fiber; and emitting at a first portion of the therapeutic light from the second end of the fiber along a direction transverse to the longitudinal axis of the fiber. In one embodiment, the step of delivering therapeutic light from the light emitting portion of the delivery device further includes emitting a second portion of the therapeutic light from the second end of the fiber along a direction substantially parallel to the longitudinal axis of the fiber. In one embodiment, the method further includes: directing the first portion of therapeutic light towards the interface; and directing the second portion of light into the hypodermis. In another embodiment, the therapeutic light includes laser light. In yet another embodiment, the therapeutic light includes light having a wavelength in the visible or near-infrared. In one embodiment, the treatment light has a wavelength of about 1440 nm. In another embodiment, the delivered therapeutic light has a total power in the range of 4 W to 20 W. In even another embodiment, the delivered therapeutic light has a total power of about 8 W. In yet another embodiment, the delivered therapeutic light has a power density in the range of about 200 W/cm̂2 to about 20,000 W/cm̂2 at the target region. In one embodiment, the step of delivering therapeutic light from the light emitting portion of the delivery device includes delivering a series of light pulses. In some embodiments, the series of pulses includes a pulse having a duration of about 0.5 ms, or in the range of about 0.1 ms to about 1.0 ms. In some other embodiments, the series of pulses has a repetition rate of about 40 Hz, or in the range of about 10 to about 100 Hz. In one embodiment, the optical delivery device includes at least one sensor, and further including: using the at least one sensor, generating a signal indicative of at least one property of the delivery device or the target region; and controlling the delivery of therapeutic light based on the sensor signal. In another embodiment, the property of the delivery device or the target region includes at least one selected from the list consisting of: a position of the optical delivery device, a movement of the optical delivery device, temperature of the optical delivery device, a tissue type in the vicinity of the optical delivery device, an amount of energy delivered by the optical delivery device, and a temperature of tissue in the target region. In yet another embodiment, the sensor includes at least one selected from the list consisting of: a thermister, an accelerometer, and a color sensor. In one embodiment, the method further includes generating a display based on signal indicative of at least one property of the delivery device or the target region. In another embodiment, the display includes a temperature map of a region of the patient undergoing treatment.

In another aspect, an apparatus is disclosed for treating cellulite in a patient. The apparatus includes an optical delivery device having a light emitting portion configured to be inserted into the patient such that the light emitting portion of the device is located below the interface between the dermis and the hypodermis of the patient; and a controller to control the delivery of therapeutic light from the light emitting portion of the delivery device to heat a target region located proximal to the interface to cause thermal damage in the target region without causing substantial thermal damage to dermal and epidermal tissue located above the target region.

In another aspect, a method is disclosed for treating an area of skin located on or near the face or neck of a patient. The method includes inserting an optical delivery device into the patient such that a light emitting portion of the device is proximal to an interface between the dermis of the skin and the underlying fascia of the patient; and delivering therapeutic light from the light emitting portion of the delivery device to heat a target region located proximal to the interface to cause thermal damage in the target region without causing substantial thermal damage to dermal and epidermal tissue located above the target region. In one embodiment, the step of delivering therapeutic light from the light emitting portion of the delivery device to heat a target region located proximal to the interface includes substantially localizing the heating of the dermis to within a desired distance above the interface. In another embodiment, the desired distance is about 0.5 mm, 1.0 mm, or less. In one embodiment, the method includes heating the target region proximal the interface to a temperature of about 50° C. or more while maintaining the upper dermal and epidermal tissue located above the target region at a temperature of about 42° C. or less. In another embodiment, the target region extends along the interface, and where delivering therapeutic light from the light emitting portion of the delivery device to heat a target region includes moving the light emitting portion of the optical delivery device along the interface while delivering the therapeutic light. In one embodiment, the method further includes modulating the delivery of therapeutic light while moving the light emitting portion of the optical delivery device along the interface to form localized sub regions of thermal damage within the target region. In another embodiment, the method further includes inserting a tip of a cannula into the target region; and moving the tip of the cannula within the target region to cause mechanical damage to tissue in the region. In one embodiment, the target region includes connective tissue which connects the dermis to underlying fascia, and where the mechanical damage includes damage to the connective tissue. In another embodiment, the optical delivery device includes an optical fiber having at least a portion housed in the cannula. In yet another embodiment, the optical delivery device includes a side firing optical fiber which extends along a longitudinal axis from a first end to a second end, and where the step of delivering therapeutic light from the light emitting portion of the delivery device includes: receiving therapeutic light at the first end of the fiber; transmitting the therapeutic light to the second end of the fiber; and emitting at a first portion of the therapeutic light from the second end of the fiber along a direction transverse to the longitudinal axis of the fiber. In even another embodiment, the step of delivering therapeutic light from the light emitting portion of the delivery device further includes emitting a second portion of the therapeutic light from the second end of the fiber along a direction substantially parallel to the longitudinal axis of the fiber. In one embodiment, the method further includes directing the first portion of therapeutic light towards the interface; and directing the second portion of light into the underlying fascia. In another embodiment, the therapeutic light includes laser light. In yet another embodiment, the therapeutic light includes light having a wavelength in the visible or near-infrared. In one embodiment, the treatment light has a wavelength of about 1440 nm. In another embodiment, the delivered therapeutic light has a total power in the range of 4 W to 20 W. In yet another embodiment, the delivered therapeutic light has a total power of about 8 W. In one embodiment, the delivered therapeutic light has a power density in the range of 200 W/cm̂2 to 20,000 W/cm̂2 at the target region. In another embodiment, the step of delivering therapeutic light from the light emitting portion of the delivery device includes delivering a series of light pulses. In some embodiments, the series of pulses includes a pulse having a duration of about 0.5 ms, or in the range of about 0.1 ms to about 1.0 ms. In some other embodiments, the series of pulses has a repetition rate of about 40 Hz, or in the range of about 10 to about 100 Hz. In some embodiments, the optical delivery device includes at least one sensor, and further including: using the at least one sensor, generating a signal indicative of at least one property of the delivery device or the target region; and controlling the delivery of therapeutic light based on the sensor signal. In one embodiment, the property of the delivery device or the target region includes at least one selected from the list consisting of: a position of the optical delivery device, a movement of the optical delivery device, temperature of the optical delivery device, a tissue type in the vicinity of the optical delivery device, an amount of energy delivered by the optical delivery device, and a temperature of tissue in the target region. In another embodiment, the sensor includes at least one selected from the list consisting of: a thermister, an accelerometer, and a color sensor. In one embodiment, the method further includes generating a display based on signal indicative of at least one property of the delivery device or the target region. In another embodiment, the display includes a temperature map of a region of the patient undergoing treatment.

In another aspect, an apparatus is disclosed for treating an area of skin located on or near the face or neck of a patient. The apparatus includes an optical delivery device having a light emitting portion configured to be inserted into the patient such that a light emitting portion of the device is proximal to an interface between the dermis of the skin and the underlying fascia of the patient; and a controller to control the delivery of therapeutic light from the light emitting portion of the delivery device to heat a target region located proximal to the interface to cause thermal damage in the target region without causing substantial thermal damage to dermal and epidermal tissue located above the target region. In one embodiment, the apparatus further includes a temperature map display.

In another aspect, a thermal surgical apparatus is disclosed. The apparatus includes a handpiece comprising a hollow cannula extending from the handpiece to a distal end, the distal end of the cannula having an outer surface comprising a recess; an optical fiber extending at least partially along the hollow cannula to the distal end and configured to deliver therapeutic light from a therapeutic light source to a treatment region located proximal the distal end of the cannula; and a temperature sensor located at least partially within the in the recess. In one embodiment, the apparatus further includes a thermally non-conductive inner material layer disposed between the thermister and the outer surface of the cannula. In another embodiment, the thermally non-conductive material layer substantially thermally insulates the temperature sensor from the outer surface of the cannula. In yet another embodiment, the insulating material includes at least one material from the list consisting of: a plastic, a polymer, polystyrene, and an adhesive material. In one embodiment, the apparatus further includes an outer material layer disposed on the outer surface of the cannula to secure the temperature sensor within the recess. In another embodiment, the outer material layer includes a sleeve disposed about at least a portion of the outer layer of the cannula to secure the temperature sensor within the recess. In even another embodiment, the outer material layer includes a thermally conductive material. In yet another embodiment, the thermally conductive material includes at least one material from the list consisting of: a metal, a metal foil, a thermally conductive polymer, a thermally conductive plastic, and a thermally conductive silicone. In one embodiment, the outer material layer has higher thermal conductivity than an inner material layer disposed between the thermister and the outer surface of the cannula. In another embodiment, the temperature sensor is a thermister. In even another embodiment, the thermister has a characteristic size of about 1 mm or less. In yet another embodiment, the thermister is characterized by a response time of about 250 ms or less. In one embodiment, the apparatus further includes a processor in communication with the temperature sensor to receive a signal from the sensor indicative of a temperature in the treatment region and control the delivery of therapeutic light from the therapeutic light source through the optical fiber. In another embodiment, the handpiece includes at least one additional sensor configured to in communication with the processor, and where: the additional sensor is configured to generate a signal indicative of at least one property of the handpiece or the treatment region; and the processor is configured to control the delivery of therapeutic light to the treatment region based on the sensor signal. In one embodiment, the property of the hanpiece or the target region includes at least one selected from the list consisting of: a position of the handpiece, a movement of the handpiece, a temperature of the handpiece, a tissue type in the vicinity of the distal end of the cannula, an amount of energy delivered to the target region, and a temperature of tissue in the target region. In another embodiment, the sensor includes at least one selected from the list consisting of: a thermister, an inertial sensor, an accelerometer, a gyroscope, and a color sensor. In yet another embodiment, the distal end of the cannula includes at least one suction port. In yet another embodiment, the recess includes a slot in the cannula. In even another embodiment, substantially the entire temperature sensor is housed within the recess. In one embodiment, at least a portion of the optical fiber is located within the hollow cannula. In another embodiment, the hollow cannula includes a suction cannula, and further comprising a treatment cannula housing at least a portion of the optical fiber.

### BRIEF DESCRIPTION OF THE DRAWINGS

The foregoing will be apparent from the following more particular description of example embodiments of the invention, as illustrated in the accompanying drawings in which like reference characters refer to the same parts throughout the different views. The drawings are not necessarily to scale, emphasis instead being placed upon illustrating embodiments of the present invention.

FIG. 1 is a schematic of a laser surgical system

FIG. 1A is an exploded view an embodiment of the accelerometer in a device of the present invention;

FIG. 2 illustrates a device of the present invention applied to a treatment area during a treatment;

FIG. 3A shows a feature in an embodiment of the device translating acceleration in one, two, or three axes and FIG. 3B shows an embodiment of the accelerometer mounted on to a device of the present invention;

FIG. 4 is a schematic illustration of a filter and an input amplifier in an embodiment of a translator processing circuit in the present invention;

FIG. 5 is a schematic diagram for total speed estimation in the speed vs. power application;

FIGS. 6A and 6B illustrate a mode of power output to reduce thermal shock to a portion of the treatment area and to provide a more even energy deposition throughout the treatment area;

FIG. 7 is a graph illustrating minimum speed vs. power curve;

FIG. 8 is a graph illustrating the speed of the device in terms of power output and repetition rate of pulses by the device;

FIG. 9 is a graph illustrating offset speed vs. power curve;

FIG. 10 illustrates a mode of plotting three-axis positions in a three-dimensional Cartesian plane in the power vs. different-in-position application;

FIG. 11 illustrates a two-dimensional map of a treatment area that represents the treated and untreated portions thereof;

FIGS. 12A-12D illustrate overlapping pulses and the mode of accounting such overlapping pulses for the map of the treatment area;

FIGS. 13A-13C graphs illustrating adiabatic temperature rise in the treatment area by 1064 nm, 1320 nm, and 1400 nm sources, respectively;

FIG. 14 illustrates a three-dimensional coordinate including a physical node within an interstitial target and a plot of Eᵢₙ, vs. propagation distance.

FIG. 15 illustrates an embodiment of a surgical system that includes embodiments of a device and a photodetector sensor pad.

FIG. 16 is a graph illustrating multiple wavelengths used in the doping beam.

FIG. 17 illustrates an embodiment of a user interface display that is in communication with a photodetector sensor pad.

FIG. 18 shows a surgical device featuring a thermal sensor.

FIG. 19 shows a surgical device featuring a thermal sensor.

FIG. 20 shows embodiments of surgical devices featuring a thermal sensor.

FIG. 21 shows a feedback loop for controlling a surgical device.

FIG. 22 shows a feedback loop for controlling a surgical device.

FIG. 23 shows a schematic illustrating temperature-position mapping for a surgical device.

FIG. 24 shows a surgical device featuring an IR thermal sensor.

FIG. 25 shows a surgical device featuring an IR thermal sensor.

FIG. 26 shows a graph of transmission properties of anti-reflection coated ZnSe.

FIG. 27 shows a tissue type sensor.

FIG. 28 shows a tissue type sensor.

FIG. 29 shows a tissue type sensor featuring a sense waveguide.

FIG. 30 shows a dual color tissue type sensor.

FIG. 31 shows an electronic circuit for use in a tissue type sensor.

FIG. 32 shows an electronic circuit for use in a tissue type sensor.

FIG. 33 shows a response curve for a color photodetector.

FIG. 34 shows an optical energy delivery device positioned blow the dermal and hypodermal interface.

FIG. 35 shows an optical energy delivery device with a side firing beam positioned blow the dermal and hypodermal interface.

FIG. 36 shows an optical energy delivery device with a side firing beam positioned blow the dermal and hypodermal interface and delivering energy to a target region at the interface.

FIG. 37 shows the same device, when articulated, delivering energy to an expanded target region.

FIG. 38 shows the device emitting pulsed energy, creating discreet target regions of thermal energy at the interface.

FIG. 39 shows the device including a temperature control sensor.

FIG. 40 shows an ultrasound of a patient before and one month following treatment of adipose herniations.

FIG. 41-46 show the effect of temperature sensing and control means incorporated into the device, and their effect on reducing temperature spikes at the treatment site.

### DETAILED DESCRIPTION

A description of example embodiments of the invention follows.

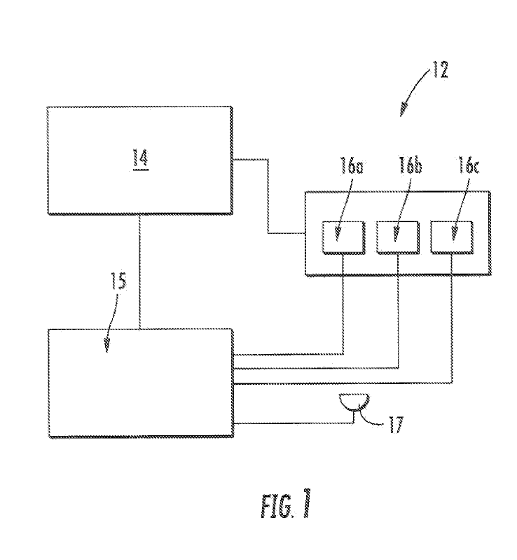

FIG. 1 shows a laser surgical system 10 featuring several safety and control features of the type described herein. System includes a handpiece 12 adapted to be handheld by a clinician or other operation, and to deliver therapeutic laser energy from laser source 14 to a treatment area (e.g. via an optical fiber). Controller 15 operates to control the delivery of therapeutic laser energy, e.g. by allowing or inhibiting the transmittal of light from source 14 to the treatment area or by controlling one or more laser parameters such as intensity, wavelength, pulse rate, etc. Handpiece 12 includes multiple sensors 16a, 16b, and 16c of differing types. For example, in the embodiment show sensor 16a is an accelerometer, sensor 16b is a temperature sensor, and sensor 16c is a tissue type sensor.

Sensors 16a-c are coupled to controller 15, which can process the outputs of the signals to determine information about the ongoing treatment. Controller 15 can process information measured by the sensors 16a-c and control laser 15 based on the processed information. Information from each of the sensors 16a-c may be used separately, or combined to provide a wealth of real time information about the area undergoing treatment. This information can be displayed to the clinician, or used to automatically control laser 15 to, for example, provide a desired dose profile across the treatment area or to inhibit laser 15 in the event that a dangerous condition (e.g. overheating or a portion of the treatment area) is detected. In some embodiments, information from the sensors 16a-c may be used to confirm each other, thereby providing enhanced reliability and safety

In some embodiments, an additional sensor 17, located external to handpiece 12 also provides information about the area of tissue undergoing treatment. For example, sensor 17 may be an infrared camera or other type or IR sensor which measures the temperature of the tissue undergoing treatment, or adjacent/related tissue (e.g. the outer surface of the skin overlaying the tissue undergoing treatment.).

FIG. 1 describes the device 100 for in vivo surgical applications. The device 100 comprises an apparatus 115. The apparatus 115 can be adapted to be handheld by a clinician (e.g., a surgeon) and includes an energy source 105. An energy delivery component 110 can be coupled to the energy source 105 and the apparatus 115 to deliver energy to a treatment area (not shown). The term “treatment area” can include any portion of a patient's body. Examples of a treatment area can include interstitial targets situated within a patient's body but also portions of the skin surface. In one embodiment, the energy delivery component 110 is an optical fiber. The energy delivery component 115, is threaded through the apparatus 115 and a sleeve 130, reaching to the tip 135. During a procedure, the portion of the energy delivery component 110 covered by the sleeve 130 is applied to the treatment area. The device 100 can further include an accelerometer 120 that is coupled to the apparatus 115 for measuring inertial acceleration. In one embodiment, the energy delivery component 110 can be an optical fiber.

The energy source 105 can be configured to provide least one of a suction energy, a light energy, a radiofrequency energy, sonic 9 e.g. ultrasound) energyand an electromagnetic radiation. In one embodiment, the energy source comprises a laser light. The laser light can comprise laser radiation. Yet in another embodiment, the laser radiation comprises a laser pulse (e.g., Nd:YAG laser). In this embodiment, the energy source comprises a laser. In one embodiment, the radiofrequency energy can comprise a radiofrequency (RF) pulse. Yet in another embodiment, the electromagnetic radiation comprises ultraviolet (UV) light.

When a pulse is delivered to the treatment area, the wavelength of a pulse also plays a factor to the amount of power applied to the target. For example, a 1440 nm wavelength pulse is more highly absorbed by, for example, fat tissue than an equivalent power 1320 nm wavelength pulse.

In certain embodiments, the device 100 can include an accelerometer 120 secured to the energy delivery component 110. The accelerometer 120 can be mounted to or within the apparatus 115 in fixed relation with respect to the energy delivery component 110. The accelerometer 120 generates an electrical signal indicative of the motion of the energy delivery component 110 in at least one direction and as many as three orthogonal directions. The electrical signal from the accelerometer 120 can be sent to a processor 125 for controlling the energy source 105, such that the operation of the energy source 105 is controlled, at least in part, by the movement of the apparatus 115.

In certain embodiments, the processor 125 can be programmed such that the energy delivery component 110 only operates when the apparatus 115 (and thus the energy delivery component 110) is in motion. When the accelerometer 120 indicates that the apparatus 115 and the energy delivery component 110 are stationary, the output of the energy source 105 ceases. This provides a safety function because it would prevent the energy delivery component 110 from delivering more than the optimal amount of the energy in rapid succession to the same portion of the treatment area, thereby preventing undesirable thermal damage. Furthermore, in one embodiment, the safety function of the device 100 can include at least a control that provides a warning feedback when the apparatus 115 is moving below a critical minimum speed. Alternatively or in combination with the safety function, the device 100 can include a control for stopping the function of the energy source 105 when the energy delivery component 110 is moving below a critical minimum speed.

In certain embodiments, the energy source emits a beam, which can be pulsed. For example, if the energy source delivers a laser light, the energy source is enabled to control the rate of a laser pulse. The energy source is configured to manipulate one or more parameters to control the amount of the total energy directed to the treatment area. In one embodiment, the energy source can control a power per pulse, a pulse duration, a pulse repetition rate, or a combination thereof. While keeping the total power directed to the treatment area constant in a time duration, the energy source is configured to increase or decrease the power per pulse, the pulse duration, the pulse rate or a combination thereof. In one embodiment, the energy source further includes a control system that is configured to control the rate at which the energy source generates pulses of each energy pulse in response to the feedback provided by the accelerometer. Thus, a device (and thus an energy delivery component) moving at a slow speed would deliver less energy directed to the treatment area. Conversely, a device moving at a higher speed would deliver more power. In one embodiment, the control system can be configured to emit energy pulses only when the device is in motion, and at a power that is modulated in accordance with the device motion in all three axes. In another embodiment, the energy source is enabled to control the rate of the energy pulse in relation to: the wavelength of a pulse, a speed of the energy delivery component, a tissue of the treatment area, fluence setting, propagation distance, or a combination thereof. The fluence setting referred herein determined whether 100% of the power is applied. The term “fluence” herein refers to a laser term meaning Joules/cm².

In certain embodiments, the device comprises a detector that is coupled to the energy delivery component for detecting the reaction by the treatment area in response to the treatment. In one embodiment, a sensor can be coupled to the energy delivery component to measure the physical change of the treatment area, in response to the energy directed thereto. In another embodiment, a detector can be coupled to the energy delivery component for detecting radiation transmitted back through the energy delivery component from the treatment area. For example, the detector detects near infrared radiation that travels down the energy delivery component from the treatment area, in the reverse direction of the energy pulses. The detected near infrared radiation can be used to monitor the temperature of the tissue in the treatment area and to regulate the operation of the energy source. Yet in another embodiment, the device can be programmed to provide a warning when the detected radiation indicates that the temperature of the tissue exceeds a pre-determined temperature. The device can further be programmed to prohibit operation of the energy source when the detected radiation indicates that the temperature of the tissue exceeds a predetermined temperature. For example, the energy source operates in a pulsed mode, and the near infrared radiation from the treatment area is detected during the delay period between successive treatment pulses. Even for a continuous wave source, the treatment beam and diagnostic beam could be modulated, such that the duty cycle of the continuous wave treatment beam was close to unity.

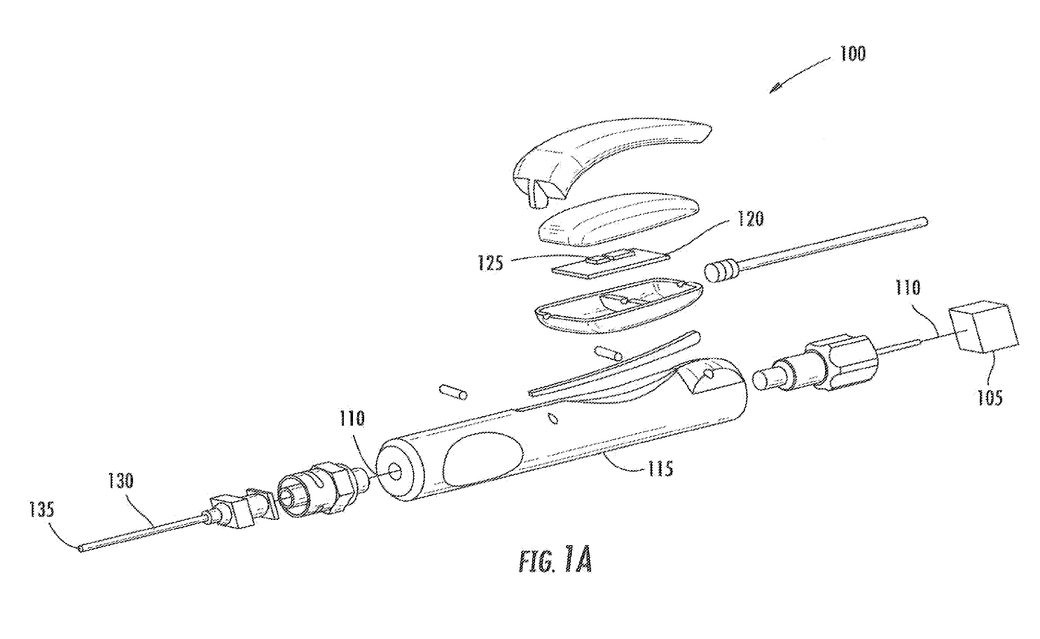

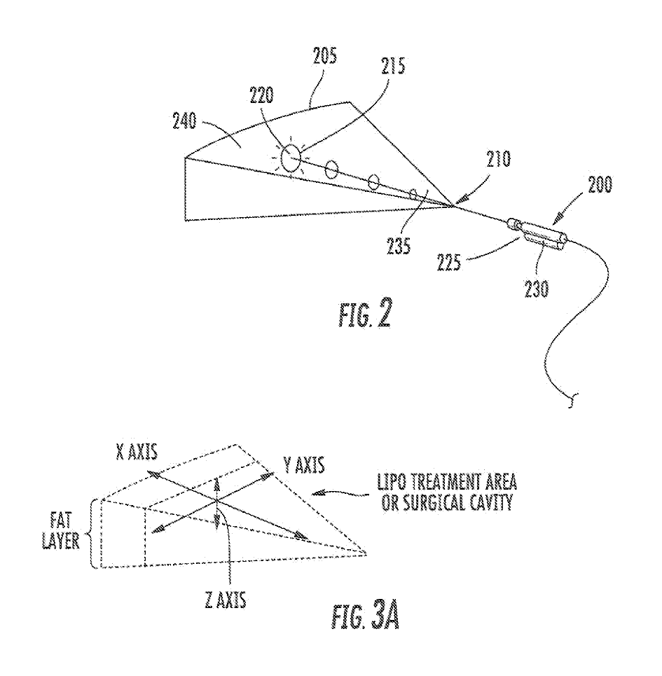

FIG. 2 shows a method how a device of the present invention can be applied. The device 200 is inserted in to a treatment area 205 (e.g., fat tissue) through an incision 210 made on the skin of a patient. As the energy delivery component 215 is inserted and moved further into the treatment area 205, the energy delivery component 215 is configured to direct one or more sequential pulses in a predetermined rate to the treatment area 205. During the procedure, much of the absorption and heating occurs in tissue immediately adjacent to the tip 220 of the energy delivery component 215. As the clinician moves back and forth, the device 200, and, thus, the energy delivery component device 215 in the treatment area 205, the energy source (not shown) provides the energy by emitting the one or more sequential pulses, distributing and breaking up tissue cells (e.g., fat cells).

In certain embodiments, the energy source is configured to modulate the amount of the energy directed to the treatment area 205 in relation to the position of the energy delivery component 215. In another embodiment, the energy source is configured to modulate the amount of the energy directed to the treatment area 205 in relation to a feedback provided by the accelerometer 230 regarding the amount of the energy delivered to a physical location within the treatment area 205.

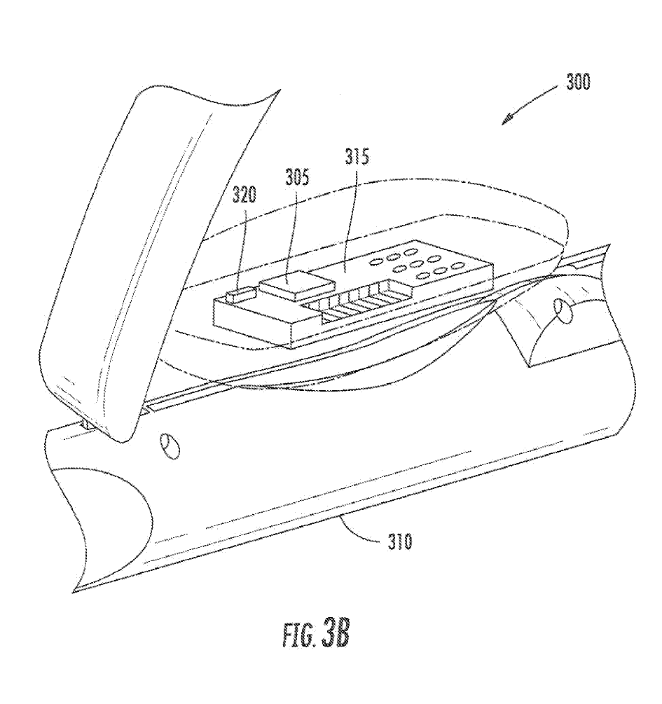

In one embodiment, as shown in FIGS. 3A and 3B, the device 300 includes a three-axis accelerometer 305 located in the laser/surgical hand piece 310 and a translator processing circuit 315, which translates acceleration into speed and/or position feedback to the operator, configured with algorithms for manipulating power or the amount of the energy output to be directed to the treatment area. The processing circuit 315 is coupled to the accelerometer 305 and determines dosimetry of the energy directed to the treatment area (not shown). The term “dosimetry” refers to the calculation of the energy dose in matter or tissue resulting from the exposure to the energy. As such, in relation to the speed and/or position feedback, the device 300 can control the power, and the amount of the energy directed to the treatment area.

In certain embodiments, the device of the present invention includes a processor coupled to an accelerometer for processing a feedback from the accelerometer and for controlling the amount of energy directed to the treatment area. In one embodiment, the device includes a power vs. speed application. In this application, the power directed to the treatment area is controlled in relation to the speed feedback. The accelerometer provides outputs, which are filtered, scaled and integrated to obtain one, two or three axes speed feedback. When the speed feedback is provided for two or three axes, the direct current (DC) component of the accelerometer 305 output can be configured to be blocked for correcting the drifts by the energy delivery component. As such, when the DC component of the accelerometer 305 is blocked, the processing circuit 315 accumulates dynamic accelerations to provide overall value for the speed, including either + or − magnitude of the speed.

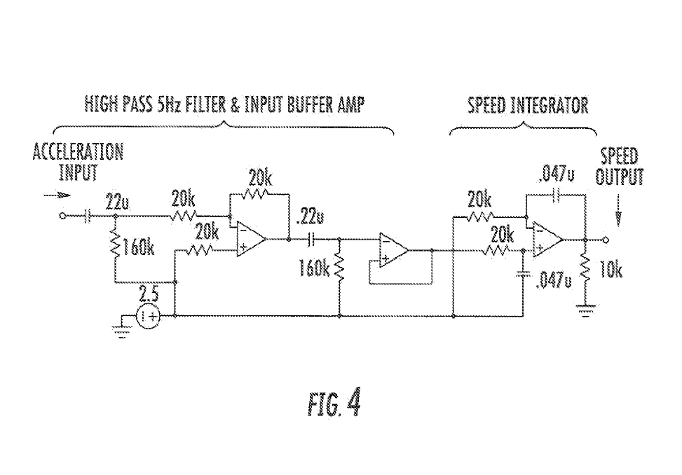

The translator processing circuit 315 includes both analog and digital elements. The three channels of the speed feedback by the accelerometer 305 are provided to the translator processing circuit 315 via a filter such as a DC blocking pass filter (with, for example, ˜5 Hz cutoff) followed by an adjustable gain input amplifier as show in FIG. 4. The input amplifier can be also offset the acceleration signals to allow for bi-directional acceleration. Through these means, the constant or static DC acceleration due to the gravity is blocked, and dynamic or changing accelerations are passed to the accelerometer to be scaled and integrated to obtain the speed feedback. Furthermore, changes in orientation of the device such as the one indicated as 300 in FIG. 3 or angles will cause the static gravity acceleration vector to be re-distributed amongst all three axes and thus to the acceleration signals because the signals are dependent on the angles of the three axis reference frame with respect to gravity.

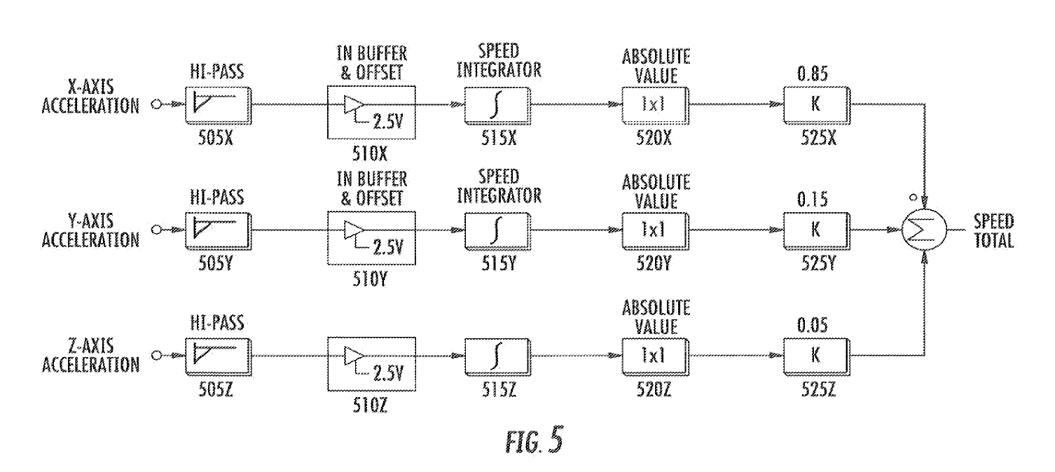

In certain embodiments of the power vs. speed application, the accelerometer is configured to provide a combined three-axis composite speed feedback. Based on the combined speed feedback, the power output directed to the treatment area can be then throttled or adjusted. Because each speed signal represents velocity along a different axis, it is not possible to simply sum the speed values from the three axes. For example, a negative speed value in the X-axis direction would subtract from a positive speed value in the Y-axis or in the Z-axis. As such, the accelerometers of the present invention can be configured to provide a quasi speed total value by taking the absolute speed value in each axis independently and then summing the absolute values from all the axes as shown in FIG. 5. FIG. 5 demonstrates one example how the devices of the present invention can provide the combined three-axis composite speed feedback. In step 505 x, y, z, the acceleration signal from each axis is measured. In steps 510 x, y, z and 515 x, y, z, the input amplifier and the acceleration signals are offset and subsequently integrated, generating speed values. The speed values from each axis are then converted to absolute values in step 520 x, y, z. In step 525 x, y, z, each of the absolute values for the speed is then weighted and summed to provide the combined three axis composite speed feedback, respectively. For example, the absolute value for the speed value for the X-axis is given the most weight, contributing 85% to the combined three axis composite speed feedback while the values of the Y and Z axes are weighted 15% and 5%, respectively. Each axis may be amplified differently to bias or emphasize the primary axis of movement for the device in the given procedure. Thus, in one embodiment, the X-axis tracks the main stroke of a procedure such as lipolysis while lateral and depth acceleration from Y and Z axis sensors by the accelerometer contribute less to the combined three axis composite speed feedback. For lipolysis, the speed in the X axis can contribute up to 80%, the Y axis up to 15%, and the Z axis up to 5% of the combined three axis composite speed feedback. To achieve 100% of the selected fluence (power out), the absolute value of the speed in all three axes are added together, the sum then must exceed the 100% fluence vs. speed threshold. If the combined three-axis composite speed feedback is less than the 100% threshold, the power out is reduced linearly in relation to the speed.

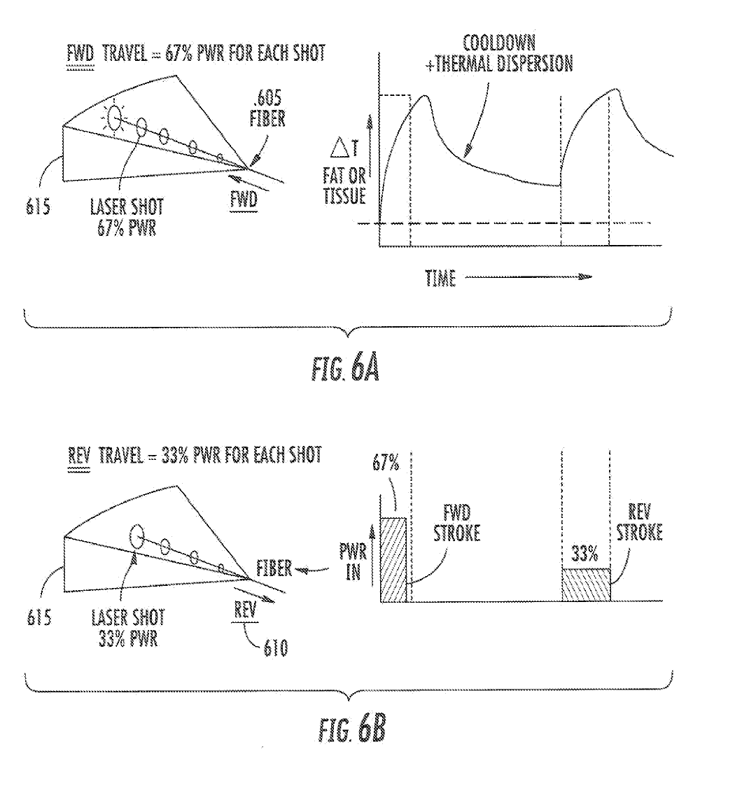

In certain embodiments, the power vs. speed feedback application can include a processor that control an energy source (e.g., the component labeled as 215 in FIG. 2) to deliver the energy to the treatment area with a direction-based power output routine. With the direction-based power output route implemented, the energy source emits varied amounts of the energy in relation to the direction which a device of the present invention moves. Such a processor is applied to evenly deliver the energy to portions of the treatment area. For example, during the forward stroke 605, 67% of the total stroke energy is deposited as shown in FIG. 6A. In FIG. 6B, the return stroke 610 deposits the remaining 33% of the total power. The idea is that some cooling/thermal dispersion time is allowed before a subsequent shot. The result is to reduce the thermal shock (fast ΔT) to the treatment area 615 while providing a more even energy deposition throughout the portion of the treatment area. Furthermore, the direction-based power output routine can be applied to side-to-side strokes.

With a power vs. speed application, the clinician can know whether and how fast the energy delivery component is moving but the clinician cannot know where the energy delivery component is moving exactly. For example, the clinician may return to the treated portion of the treatment area repeatedly (e.g., moving along the X-axis back and forth only with no speed in the Y- and Z-axes). In such case, the speed feedback allows maximum power output as long as the X-axis speed exceeds the minimum speed vs. 100% fluence limit. In one embodiment of the processor or the translator processing circuit, the processor or the translator processing is configured with an algorithm that limits the power directed to the treatment area in relation to the speed of the energy delivery component. With such algorithm, safety is greatly enhanced. Injuries due to excessive dwell time are easily prevented, and ease of learning by the operator for the optimum tempo by the device of the present invention with the power vs. speed application is enhanced. In another embodiment for safety measures, the devices of the present invention can be configured with audio feedbacks that indicate various conditions of the device and/or the treatment area. The audio feedbacks can indicate, for example: out of power, excessive temperature increase at a portion of the treatment area, proximity detection of un-targeted tissues (e.g., as determined by probe/doping beam remittance & or reflectance photo-detector) and adverse conditions (e.g., bleeding, charring).

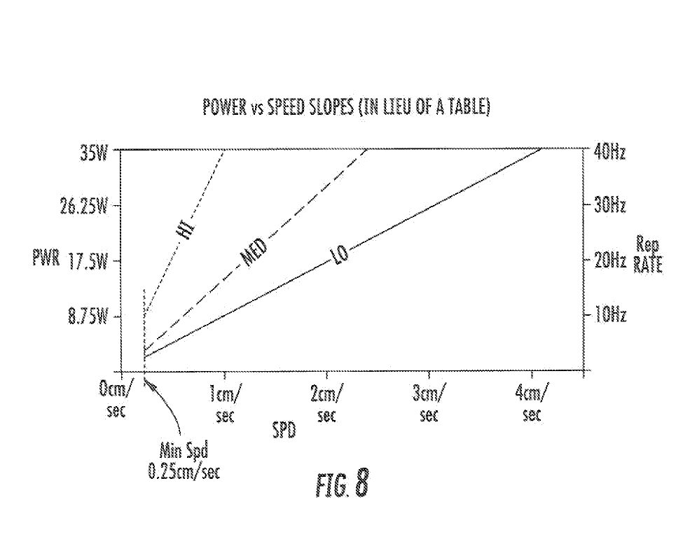

In certain embodiments, the power vs. speed application further includes a processor that implement a power limiting algorithm. The algorithm can limit or throttle power out such that the energy/unit area of the treatment area does not exceed safe thermal limits. Variables for determining how much power is safe in relation to at least one of the following: wavelength, fluence setting, tissue type (e.g., absorbance by the tissue), propagation distance and repetition rate. For example, as described in FIG. 6, a basic curve would require twice the minimum speed for a 2 Hz setting as compared to a 1 Hz setting because a power output is doubled at the 2 Hz setting. A different slope for each repetition rate is indicated in FIG. 8. FIG. 8 illustrates that the power directed to the treatment area and/or the repetition rate of pulses is adjusted in relation to the speed of the device. The minimum speed curve is to prevent excessive tissue temperature rise based on estimates of at least one of the following: applied energy, tissue absorbance, cool down time, and hand piece travel speed. Furthermore, a slope correction factor can be derived from each wavelength and/or each tissue type.

In one embodiment of the power limiting algorithm, the device can include an energy source that is configured to modulate the amount of the energy emitted when the energy delivery component is within a predetermined distance from the point of entry into the treatment area. Referring back to FIG. 2, when the tip 220 revisits the physical location that has been already treated, the energy source (not shown) is configured to modulate the amount of the energy delivered to the respective portions of the treatment area so that the already treated portions are not burned but optimally treated with an appropriate amount of the energy. For example, the tip 220 comes in contact with the portions at the physical location 235 near the incision 210 more frequently than the ones in the physical location 240, which are relatively far from the incision 210. Therefore, if the portions of the physical location 235 get pulsed with the same amount of the energy every time the tip 220 makes contact, these portions would be burned in time. To prevent this type of undesired overexposure of the energy to the portions near the incision 210, the energy source is configured to modulate the amount of the energy delivered to the portions within a predetermined distance from the incision 210 and put a limit on the amount of the energy directed thereto.

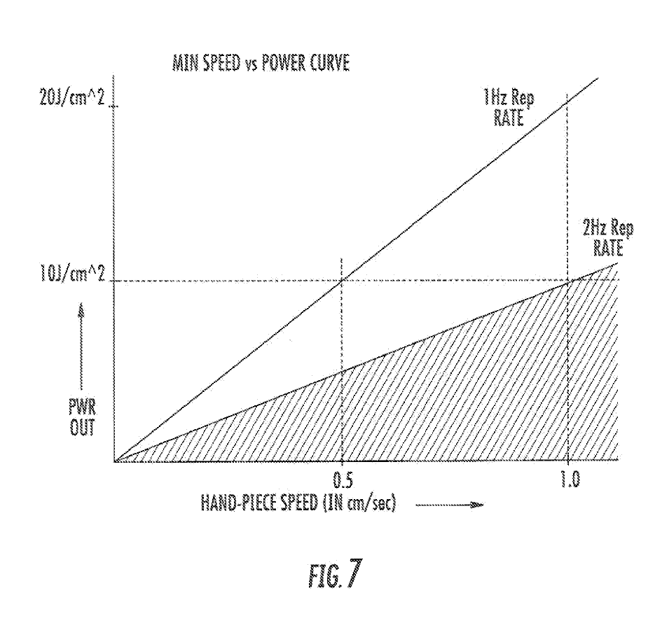

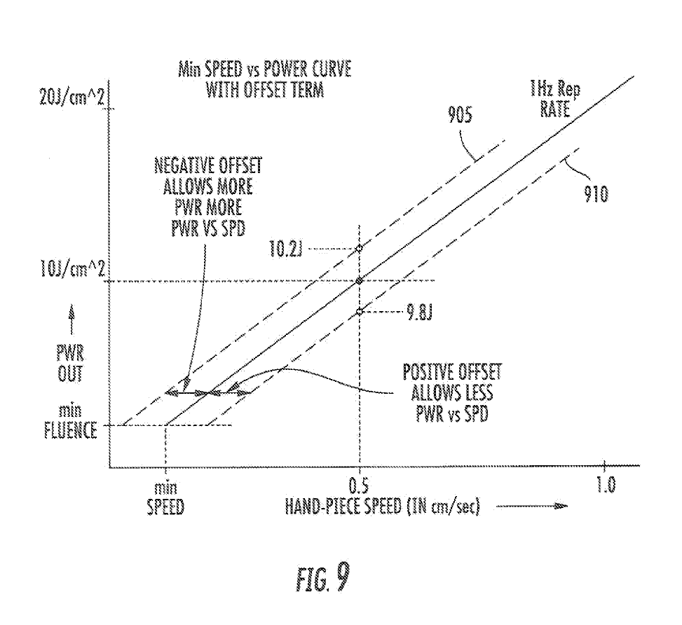

In certain embodiments of the power vs. speed application, the devices of the present invention can further include an offset mechanism, as illustrated in FIG. 7. In one embodiment, the device includes a laser light and the laser light can be throttled directly by the speed of the travel of the device. The offset mechanism allows some deviation from the speed vs. power graph provided in FIG. 9. For example, this provides the clinician the ability to fine tune the energy vs. speed slope within hard-coded safe limits to suit the specific procedure. For example, the devices can be configured to apply a negative offset to increasing power in the power vs. speed application for a 1 Hz repetition rate setting as indicated by the curve 905. Conversely, when a positive offsetting is applied, the devices are configured to emit less power as indicated by the curve 910. The laser then reduces power in relation to the speed so that the speed of travel still determines the percentage of the selected fluence to be allowed. Obviously, the selected fluence would never be exceeded regardless of the device's travel speed.

An alternative to the power vs. speed power limiting algorithm is a power vs. difference-in-position” (Δ-position) application. In this case, translation vectors are calculated from the difference-in-position in all three axes. These translation vectors defines the distance and absolute speed through three-dimensional space.

The power vs. difference-in-position power application allows a more precise control and true energy/unit area temperature rise limitation. Specifically, by tracking the absolute position of the device and simultaneously the wavelength and power out (e.g., fat tissue absorbance) a very good estimate of local temperature rise can be made.

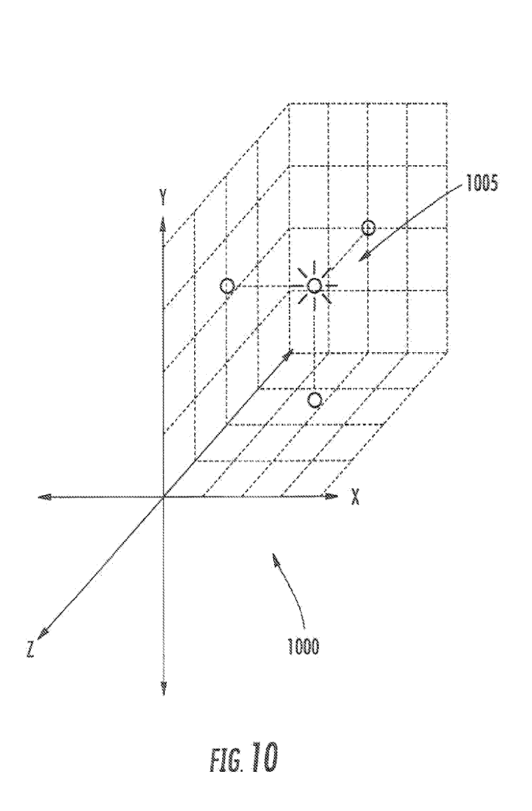

By plotting three separate position tracks, acceleration independently measured in all three axes using an accelerometer is twice integrated to yield the precise position in three-dimensional space of the interstitial target as shown in FIG. 10. The position tracks in the three axes are plotted and placed on a three-dimensional Cartesian plane 1000. The three axes converges on one point, and the plotting of the convergence of the three axes yields the actual position 1005 of the energy delivery component of the device in the present invention in the target area.

Location of each shot locked to an absolute position can be recorded throughout the procedure by creating a map of the treatment area. A simple pixel darkening display to the operator allows quick identification of missed or untreated areas. This feedback allows for a more evenly distributed energy treatment.

In certain embodiments of the power vs. difference-in-position application, the treatment area is surface portions of the patient's skin (e.g., face). Similar to a three-dimensional map of the interstitial target shown in FIG. 10, a three-dimensional topographical map displaying peaks and valleys of the skin surface portions. Prior to the treatment, the three-dimensional topographical map is produced using a two-dimension-to-three-dimension algorithm based on photos of the skin surface portions. Each point on the typographical map represents an accumulation that accounts at least one of the energy applied or Eᵢₙ, absorbance vs. or propagation distance and the time constant and continuity associated with the tissue type. During the treatment, the three dimensional topographical map is configured to indicate: the position of the energy delivery component; the amount of the energy directed to the respective portion; and/or the amount of the energy absorbed by the respective portion.

In certain embodiments of the power vs. difference-in-position application, the power directed to the treatment area is controlled in relation to the position feedback where translation is calculated from the difference in position in all three axes. This translation vector defines the distance and absolute speed in three-dimensional space. The translator processing circuit that is coupled to the accelerometer for the difference-in-position feedback application differs from the speed feedback in that gravity can no longer be disregarded. Rather, the direction of the gravity vector must be determined either mathematically or by use of a gyro (e.g., the component labeled as 320 in FIG. 3) coupled to the accelerometer in the device. The advantage of the gyro is that once aligned, at the start of a procedure, the gyro can provide precise inclination feedback, which allows the translator to subtract gravity and independently account the accelerations from each of the axis to derive speed and position. The gyro also allows for other accelerometer drift and offset compensation.

In one embodiment of the power vs. difference-in-position application, these position feedback values can be charted on a three-dimensional coordinate plane and any change in position of the energy delivery component in the three-axis coordinates. This accounting of the position allows computation of a translation vector that defines distance between points in a three-dimensional coordinate plane, travel time between points or other relevant positional data and provides absolute position as well as actual three-dimensional speed total. Another advantage of a three-dimensional coordinate plane is simplifying complex operations such as allowing for an offset vector and distance, rotation about any axis or mirror image management of position data. An example of the need for mirror image translation is such component as the apparatus 105 in FIG. 1. The component moves in mirror image coordinate plane relative to such component as the energy delivery component 110, which is within the body.

The algorithm configured with a power vs. difference-in-position application can also limit or prevent the discharge of excessive energy into an already treated spot/position. Thus, the clinician can pass over the same tissue sector multiple times while the laser throttles back the power on a pulse by pulse or millisecond basis to prevent excessive thermal rise. The less time the clinician allows for cooling of a previously treated area, correspondingly less energy is then subsequently allowed. This embodiment is illustrated in FIG. 11. While the present invention can operate two- or three-dimensionally, for the sake of explanation, FIG. 11 shows only a two-dimensional sectional map illustrating a treatment area 1100. As the clinician maneuvers within the treatment area 1100, the map records all the portions that are treated with the device 1140 and provide the clinician a view of the treatment area similar to the one shown in FIG. 11. The treatment area 1100 can be charted and divided into different sections 1110, 1115, 1120, 1125, and 1130 representing internal body cavities or treatment areas. The spots/positions 1105, 1106, 1107 are the ones that are already treated with, for example, a laser pulse and the portions 1135, 1136, 1137 are yet to be treated. As the treatment proceeds, the clinician, observing from the position based on the power vs. α-position application, can readily discern the treated portions 1105, 1106, 1107 of the treatment area 1100 from the untreated ones 1135, 1136, 1137. Thus, the clinician would then maneuver the device 1140 and move onto to treat the untreated portions 1135, 1136, 1137 of the treatment area 1100. In addition to the locations of the treated portions 1105, 1106, 1107, the map of the treatment area 1100 also shows the amount of the energy/area directed thereto and/or the amount of the energy absorbed. For example, the section 1130 being treated with, for example, more laser pulses than other sections, the map would provide an feedback indicating that the section 1130 are treated with more power/area for than other sections and that certain portions are already treated optimally. In one embodiment, the map of the treatment area can include color coding. The color coding can indicate the effects of the treatments such as the magnitude of absorbance by the portions of the treatment area. The color coding can also indicate intensity of the emitted pulses, for example, a solid red dot for many shots of pulses at certain wavelength, and a weak red dot for few shots of pulses at another wavelength.

In certain embodiments, the devices configured with a power vs. difference-in-position application discussed herein can include the safety features similar to ones discussed earlier with the speed vs. power application.

In certain embodiments, the devices configured with a power vs. difference-in-position application discussed herein can include one or more of the processors and/or power limiting algorithms that were discussed with respect to the power vs. speed applications, including one for evenly distributing the energy within treatment area, analogous to the speed feedback application as previously discussed.

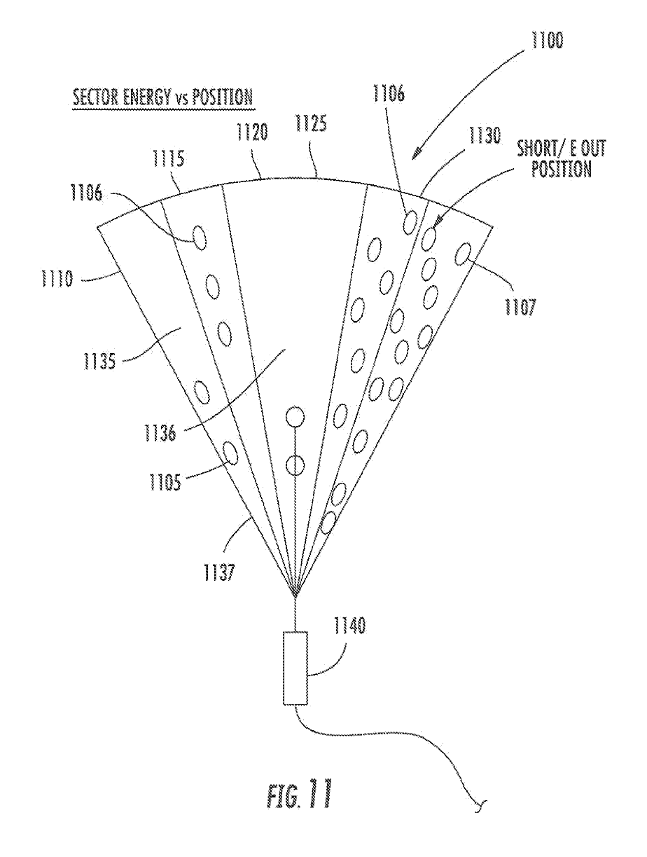

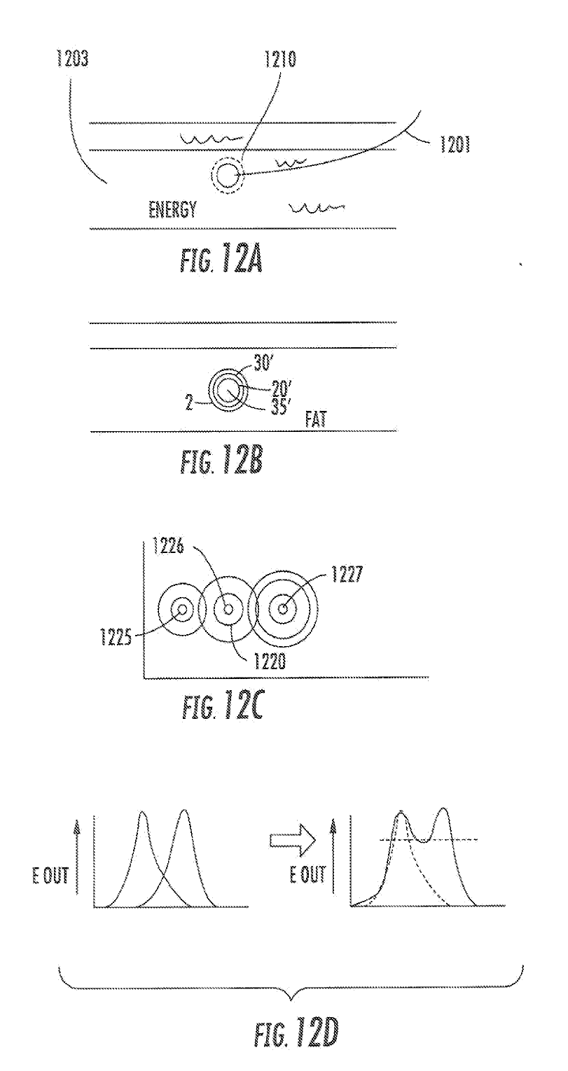

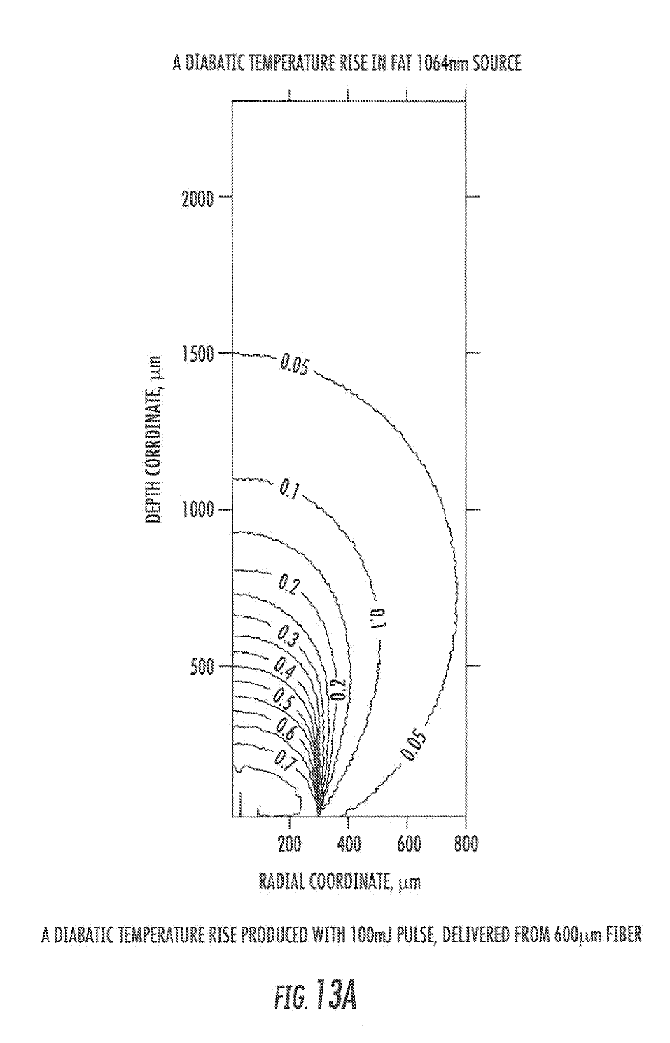

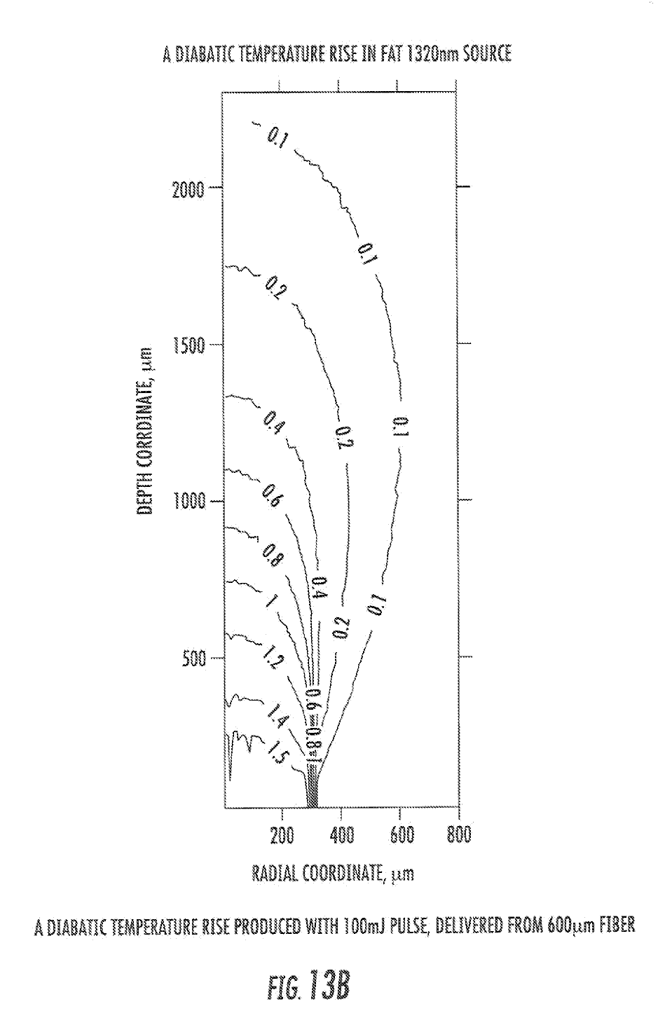

In certain embodiments, the device of the present invention further includes a processor that accounts for overlapping pulses. Each pulse propagates different distances and difference absorbance depending on the wavelength of the pulse. When the series of pulse are emitted, the wavelength absorbance and propagation distance can be overlapped as illustrated in FIGS. 12A-12D. FIG. 12A shows an energy delivery component 1201 inserted under a treatment area 1205 and delivered to an energy (e.g., a laser pulse) 1210. FIG. 12B shows the radial temperature rise from the delivered energy 1210, bringing the nearest circle to the origin of the energy 1210 being hottest to approximately 70° C. to the farthest circle at approximately to 50° C. FIG. 12C shows hotspots 1220, 1221 resulted from a series of overlapping pulses 1225, 1226, 1227. For the purpose of accounting the amount of power delivered to a hot spot, the resultant thermal energy absorbed at the hot spot 1220 can be simply added together. When the series of the pulses are emitted in different wavelengths, the total energy absorbed vs. distance of all constituent wavelengths of the pulses allows precise prediction of tissue temperature rise. FIG. 12D depicts two sequential and closely placed or overlapping shots (laser pulses) with the corresponding temperature rise vs distance. Change in temperature or ΔT for individual shots can be estimated by the adiabatic calculations, wherein the wavelength, power, target tissue absorbance and scattering effects allow calculation of ΔT with respect to distance and direction in the target tissue. Closely placed shot's wherein the resulting tissue ΔT zones overlap, have an additional accumulation of temperature due to preheating from adjacently delivered shots. Further, the ratio between the maximum ΔT and minimum ΔT with respect to distance can be defined as the “differential ΔTmax”. For example, to deposit energy and cause a very even tissue heating the “differential ΔTmax” should be minimized to provide more consistent tissue heating.

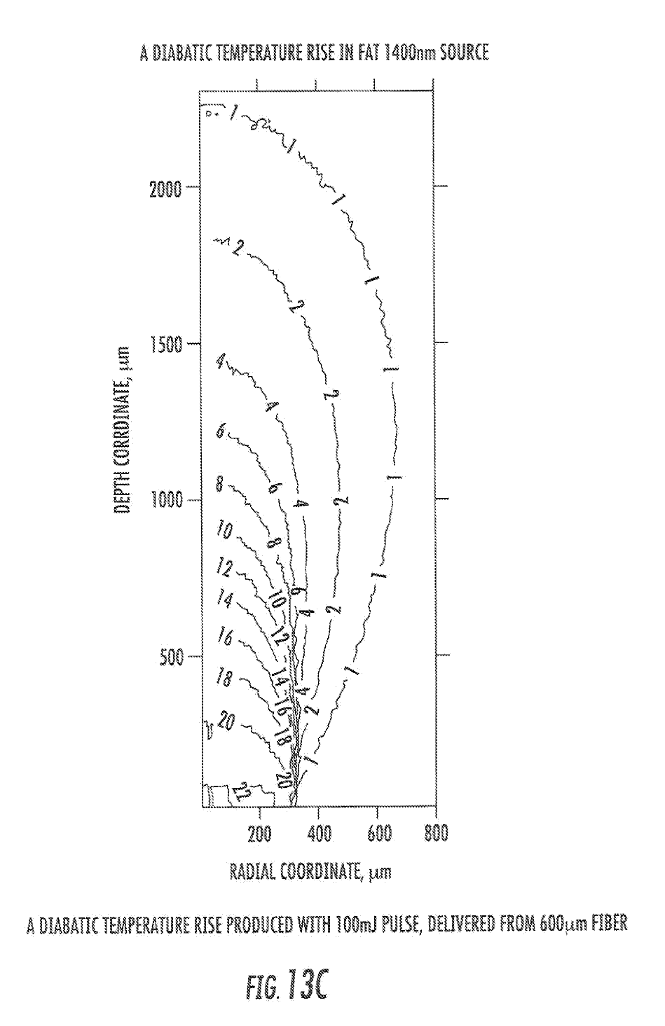

As shown in FIGS. 13A-13C, adiabatic temperature rises when a portion of the treatment area when exposed to 1064 nm, 1320 nm, and 1400 nm energy delivery components (e.g., a 600 μm fiber) delivering 100 mJ are 0.2° C., 0.81° C., and 20° C., respectively, at a 300 μm radial coordinate.

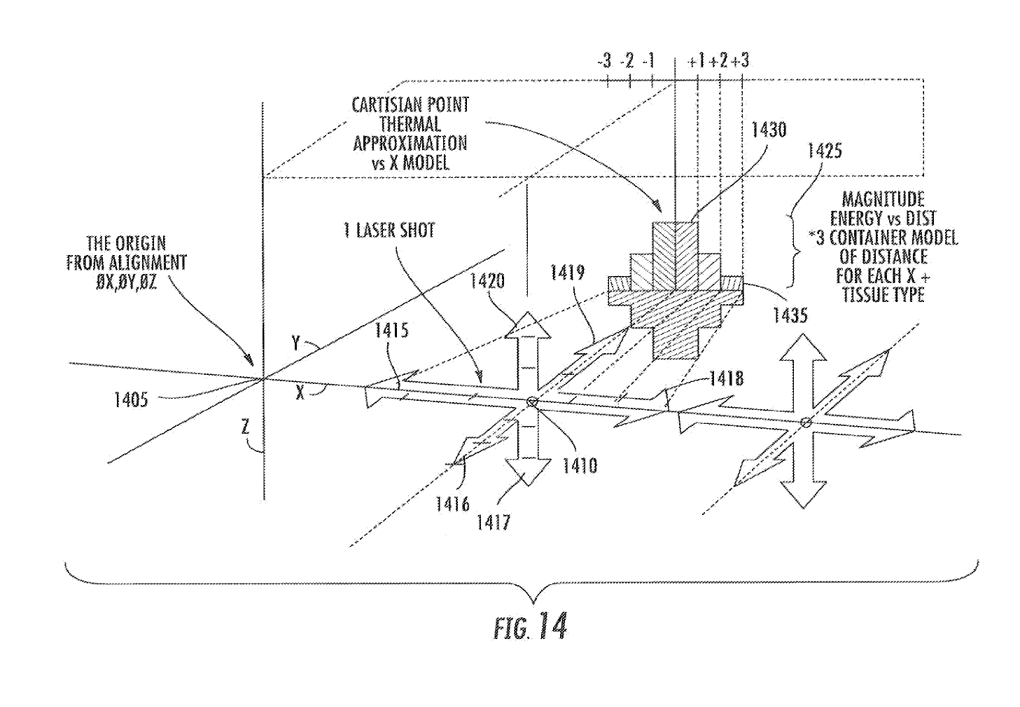

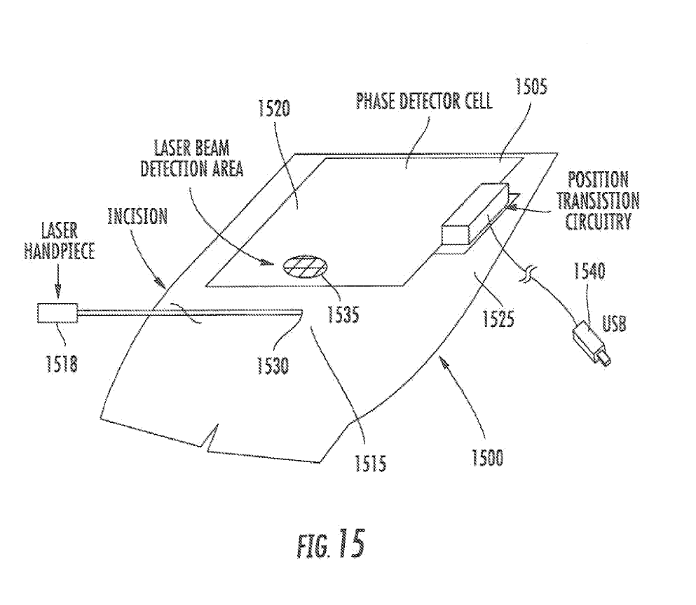

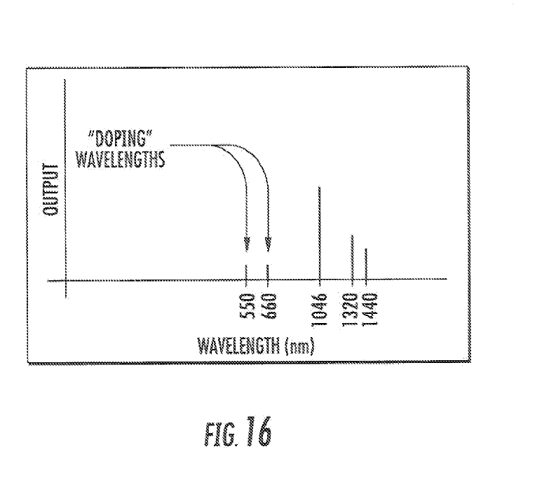

In certain embodiments of the power vs. difference-in-position application, the treatment area is an interstitial target. Using the accelerometer that is coupled to a device of the present invention, an area internal to the body can be mapped, and, thereby enabling the device to navigate the interstitial target. In one embodiment of the three-dimensional map, the point at which the energy is emitted is the origin (0ₓ, 0_{y}, 0_{z}) 1405, as shown in FIG. 14. Each physical point of the three dimensional map includes an accumulator that measures combined effect of absorbed energy within the range of the physical node represented by the accumulator when the energy in (Eᵢₙ)), for example, a laser pulse, is directed to the physical node 1410. The arrows 1415, 1416, 1417, 1418, 1419 and 1420 show the propagation distance of the Eᵢₙ to the interstitial target (the vectors of Eᵢₙ propagation are indicated in three axes to simplify math and translation). Each point on the three dimensional map represents an accumulation that accounts at least one of the energy applied or Eᵢₙ, absorbance vs. or propagation distance and the time constant and continuity associated with the tissue type. The graph shadowing the arrows are a plot 1425 of magnitude energy vs. distance. The numbers +1, +2, +3, −1, −2, and −3 indicate an arbitrary distance from the physical node 1410, +1 and −1 being the nearest. As such, the area under +1 and −1 neared the physical node 1410 provided with or absorbed the most energy, indicated by the highest peak temperature rise 1430. Conversely, the area further from the physical node 1410, for example +3 or −3 show the lowest peak temperature rise 1435.

In certain embodiments, as discussed in more detail below, doping beam or other techniques could be used to determine tissue type. For example, using 2 different wavelength low power light-emitting diode (such as in oximetry devices) allows us to distinguish color specific reflectivity or remittance. The main treatment wavelength may even be one of the doping or probing beams multiplexed into the energy delivery component. Because tissues reflect different wavelengths based on the type, the type of the tissue made up the physical node 1410 can be ascertained by a doping beam during the treatment. As the device of the present invention maneuvers within the interstitial target, the energy source can be adjusted automatically in accordance with the tissue type to provide a predetermined amount of the energy that is suitable for an optimal treatment. Furthermore, in another embodiment, the accumulator also tracks the rate of cooling at the physical node 1410 after one or more shots of the energy. As such, when the device returns to the physical node 1410, the energy source can be adjusted based on the rate of cooling to determine whether any more treatment is necessary and by how much.

The tissue discriminator or doping beam can also ascertain the location of the device in relation to the skin. If fiber approaches too close to the skin (from beneath), a suitable change in reflectivity vs. color is observed thus allowing the algorithm to shut down the laser before causing a burn, or providing a warning to the operator. In one embodiment, the doping beam is located at the tip of an energy delivery component and emits a beam which is then reflected by the tissue and detected by a sensor.

The embodiment of the device described herein are provided with an energy source that related to laser or light energy. However, these energy sources can be substituted with suction energy, as commonly used in lipolysis. In the embodiments with suction energy, an accelerometer is in communication with the suction energy source, and thus, the suction energy source can modulate an amount of the suction energy directed to the treatment area. Instead of having an energy delivery component (i.e., the component 110 in FIG. 1) that threads the apparatus (115 in FIG. 1) and the cannula (130 in FIG. 1), the cannula by itself would be applied to remove tissue or undesired bodily parts from the treatment area.

In certain embodiments of the present invention, a surgical system 1500 includes a device 1510, which is analogous to the apparatus indicated as 100, 200, 300, or the one with suction energy, and a visual display that is in communication with the device. In one embodiment, the visual display indicates the position of the a component that is analogous to the energy delivery component such as ones indicated as 315, in FIG. 3, and/or the amount of energy absorbed by a physical point of the treatment area. An example of the visual display is a photodetector sensor pad 1505 as illustrated in FIG. 14. The photo-detector sensor pad 1505 is a thin sheet containing a matrix of photodetector elements that is placed on the patient over the treatment area. In one embodiment, the sensor pad 1505 comprises a matrix of dye-based solar cells 1520 (DBSC) that can be fabricated using any known means, such as conventional silk-screen printing processes. In another embodiment, the sensor pad 1505 comprises of a matrix of DBSCs (e.g., ˜100 1 cm by 1 cm matrix). The DBSCs are fabricated on a flexible plastic material having metalized electrodes printed onto the plastic material to carry signals back to detection circuitry 1525. As shown in FIG. 15, the sensor pad 1505 is placed on the patient over the area to be treated, and detects the physical point 1535 of the tip 1530 and laser shots fired on a shot-by-shot basis. The sensor pad 1505 communicates the physical point 1535 of the tip 1530 and where shots are fired back to the laser via a data connector 1540, such as a USB connector. This information can then be displayed on a touch-screen display to aide the doctor during the procedure, and can also be used by the laser control system to disable the laser if too many shots have been fired in any one position.

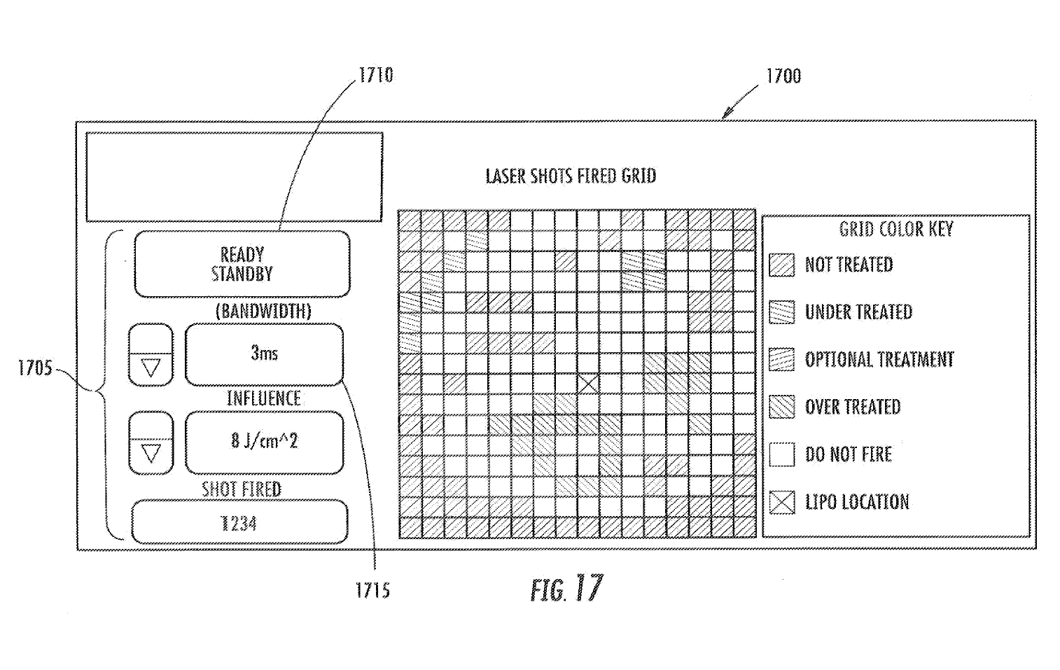

As shown in FIG. 15, as the laser is fired, the location that the laser is fired will be detected by one or a small grouping of the photodetectors 1535 which then send x, y coordinates back to the laser control system for display. The laser beam could also be doped with one or several low power constant light sources, such as light-emitting diodes, to convey the tip position back to the clinician for proper location of the tip during treatment. As shown in FIG. 16, the doping wavelengths could be, for example, 550 nm or 660 nm, or a combination of both. When multiple wavelengths are used in the doping beam, the depth of the laser hand piece tip can be determined by detecting changes in the amplitude, e.g. due to the differential scattering of the two wavelengths, of the doping beams.

The sensor pad 1505 can be a disposable component that is removed from the position translation circuitry 1525 after use and discarded. The translation circuitry 1525 can then be attached to a new sensor pad (not shown) for use in a subsequent lipolysis treatment.

The laser lipolysis system can include a user interface display 1700 as shown in FIG. 17. This display 1700 includes basic laser interface controls 1705, such as pulse width control 1710, fluence display 1715 and controls, etc. In addition, a laser shot location display 1700 can display the current location of the tip (e.g., the component 1530 in FIG. 15) as well as where on the sensor pad (e.g., the component 1505 in FIG. 15) shots have been recorded. The shot location display preferably also indicates the level of treatment that has occurred throughout the grid, such as by a color-coding of the grid. This display can be used to aide the doctor in positioning the device for the next shot and to prevent overtreatment in any one location of the treatment area.

Thermal Sensing

The following describes in greater detail thermal sensing techniques of the type described above, used alone, or in conjunction with other sensor information.

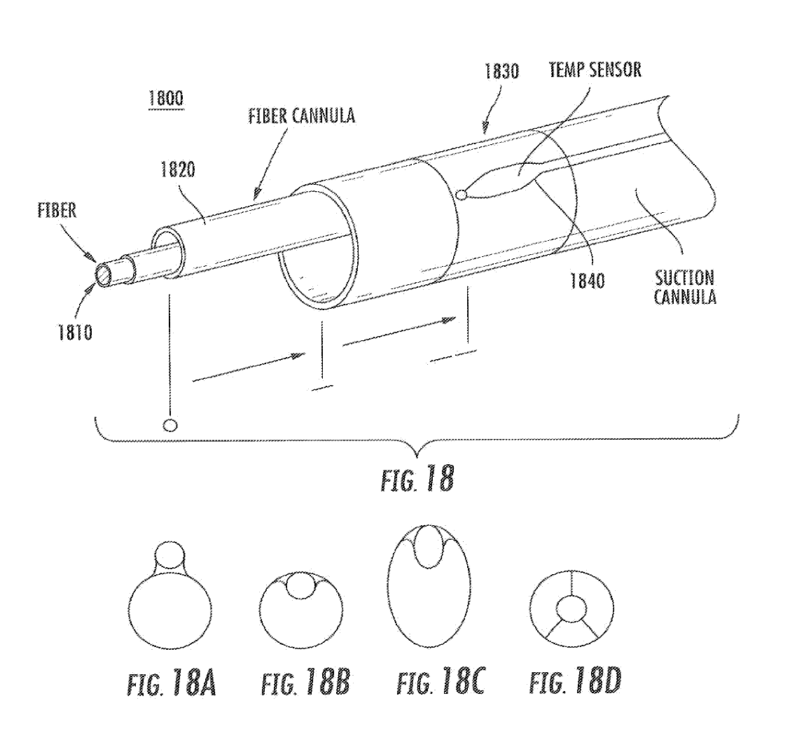

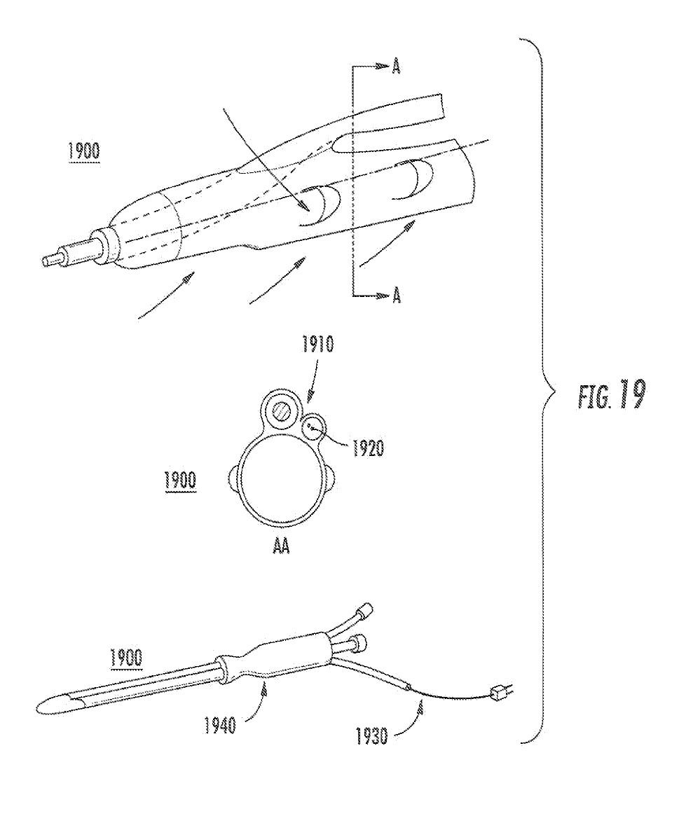

Temperature sensors may be mounted on surgical devices in any suitable fashion. For example, FIG. 18 shows a surgical probe 1800 for laser liposuction which includes an optical fiber 1810 in a fiber cannula 182. The optical fiber 1810 delivers treatment light to tissue (e.g., fat tissue). The probe also includes a suction cannula 1830 for removal of treatment by-product. A feature of this probe is a temperature sensor 1840 integral to the suction cannula. The temperature sensor 1840 is set back from the laser fiber tip. In typical embodiments, this configuration avoids localized heating of the tip of fiber 1810 and cannula 1820 leading to false readings of tissue temperature.

During a surgical procedure, tissue temperature can be read while holding the probe stationary (a short pause) within the lasing field. Based on the reading, more laser energy or cooling effort can be applied to reach the desired internal tissue temperature. In typical applications, temperature readings will fluctuate (e.g. if the probe is being rapidly reciprocated into and out of the tissue). In such cases, the temperature readings may be averaged to indicate a meaningful temperature.

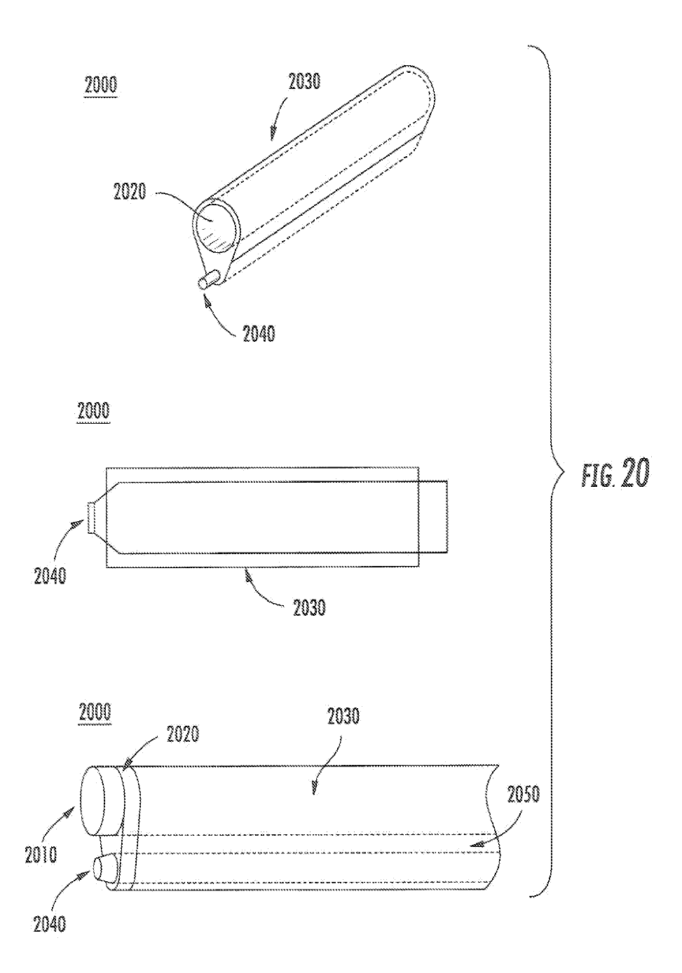

In various embodiments, any suitable temperature sensor may be included with any of a variety of surgical probe types. For example, FIG. 19 shows a surgical probe for laser liposuction featuring a separate, stainless steel cannula 1910 for the temperature sensor 1920. The temperature sensor 1920 resides in the tip of the cannula 1910, and one or more wires 1930 run up through the cannula 1910, into a hand piece 1940. The wires 1930 extend from the end of the hand piece 1940 and can be connected to a monitor or processing unit.

FIG. 20 shows an embodiment of a laser surgical probe 2000 which, unlike the embodiments shown immediately above, does not include a suction cannula. The probe includes 2000 an optical fiber 2010 for delivering treatment light placed in an inner cannula 2020 (e.g. a standard 600 μm cannula). A larger outer cannula 2030 surrounds the inner cannula 2020. A temperature sensor 2040 (e.g. a thermocouple junction) is located near the tip of the outer cannula 2030. The sensor 2040 and connecting wires extending therefrom are thermally and electrically isolated from the inner cannula 2020. For example, as shown in the lower portion of the figure, the sensor 2040 and wires may be surrounded by a thermally and electrically insulating material jacket 2050. In some embodiments, the sensor tip, wires, and insulating jacked may be autoclavable. In one embodiment, the thermister is bonded and housed to the cannula's outer surface by being blanketed in a (autoclavable, biocompatible) heat shrink.

In various embodiments, the use of a thermistor or thermocouple located within or adjacent to the cannula tip provides tissue temperature feedback to the laser. Tissue temperature feedback allows the possibility of closed loop tissue temperature control wherein the laser output (power, pulse rate, wavelength etc.) may be controlled (e.g. modulated) to effect a desired tissue temperature profile for a given procedure. For example, deep “fat busting” procedures typically place the cannula tip well out of range of surface temperature feedback techniques such as an IR camera. It is easy to unintentionally overheat deep tissue layers (e.g. beyond the temperature required for optimum safe lipo disruption). Excessive deep heating is associated with various deleterious side effects such as necrosis of blood vessels, or even thermal damage to adjacent tissue layers (muscle, fascia, etc). By employing a closed loop temperature management system optimum tissue temperatures can be maintained, simplifying the procedure for the clinician and providing improved efficacy with enhanced safety.

Another example of closed loop temperature management benefits is in skin tightening procedures where the cannula tip is placed proximal to the sub dermal layer. In essence the laser heats fat adjacent to these deeper dermal areas and said heat acts on the entire dermis to affect so called collagen remodeling (skin tightening). In some applications, a difficulty is that thermal conduction through dermal layers (to effect skin tightening) varies greatly based on skin type and thickness. Thermal gradients from deep dermis to epidermal layers may vary considerably. Thus it is possible to over heat deeper sub-dermal areas while effecting optimum surface temperatures. This may cause vascular damage and other side effects. With closed loop thermal control of deeper or sub dermal layers, a compromise between optimum epidermal temperature and sub dermal temperatures can be made.

For various applications, the optimum time constant (response rate) of any tissue contact temperature measuring device may vary. A faster response time has the advantage of actively measuring tissue temperature throughout the surgeon's treatment stroke. To accomplish this, the thermal mass of the thermistor or thermocouple should be reduced or minimized. Another possibility is to measure the treatment stroke length (e.g. using an accelerometer to measure a sign change in the velocity of the probe), divide the treatment stroke into near, mid and far “ranges” and then sample average temperature for the period the cannula tip is present in each range. This allows a slower response time thermal couple to generate a relatively precise average temperature feedback signal for each of the near, mid and far range areas. Said feedback can then be used by the laser to adjust or even out temperature accumulation through each “range” of the cannula stroke. This approach compensates for poor clinician technique.

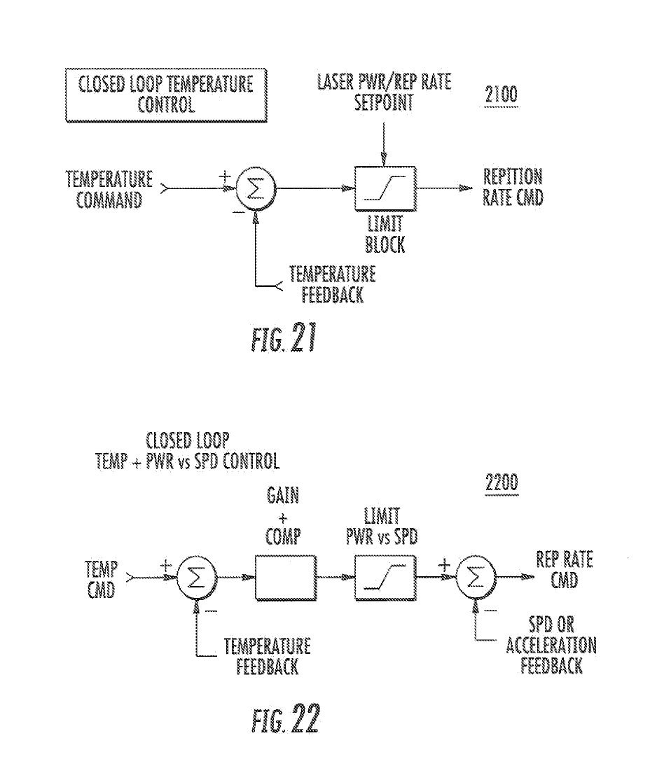

As shown in FIG. 21, in some embodiments, the closed loop control 2100 consists of a temperature control loop where a temperature error signal is derived by a summation block/difference amplifier and laser average power (or, equivalently, for pulsed lasers, variable repetition rate) acts as a limit value. Desired final tissue temperature is selected as “temperature command”. When summed with the temperature feedback from the cannula thermister a temperature error term results. This error is then gained (amplified) and compensated, the result of which is then clamped by the laser power/repetition rate setpoint limiter. The resulting output acts as a laser power, or laser repetition rate command. Operation is such that once the tissue temperature reaches the temperature command, laser output is inhibited. Regardless of temperature, the laser will not exceed the laser power/rep rate limit value.

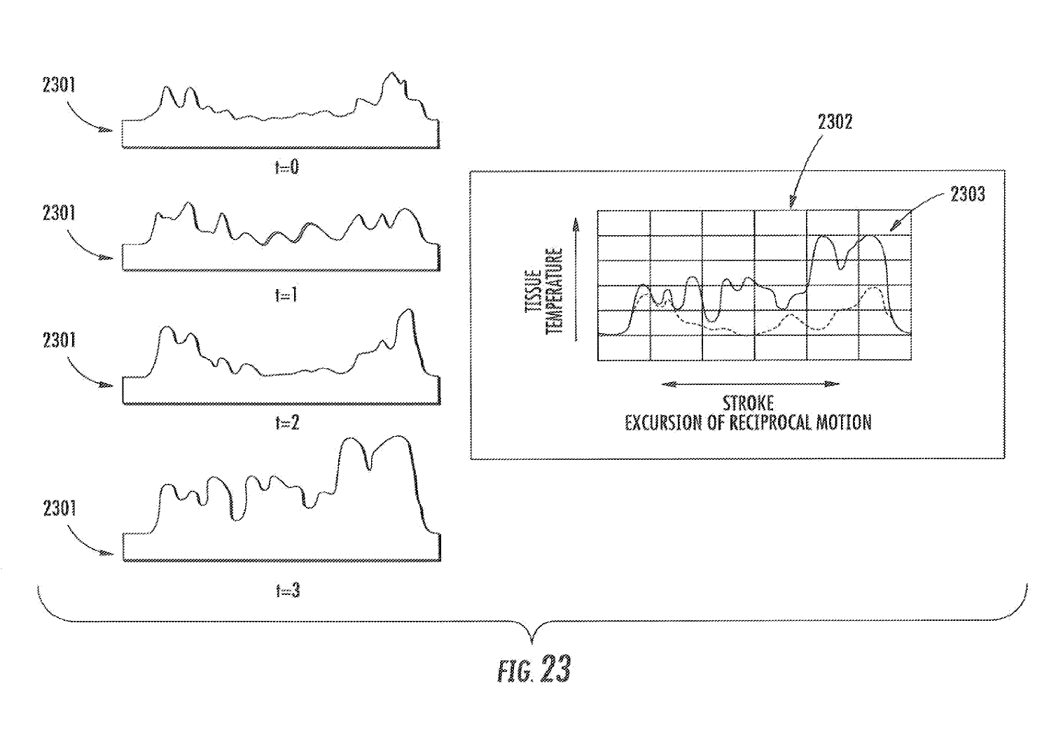

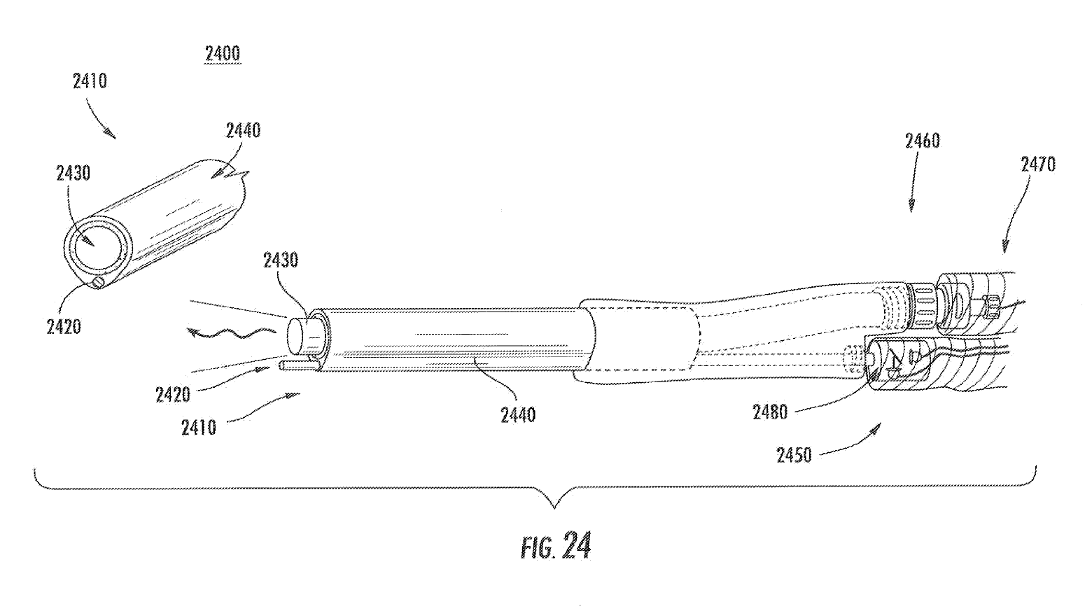

As shown in FIG. 22, in some embodiments, control loop 2200 includes an outer tissue temperature loop combined with an inner laser power vs. speed (or velocity) laser control loop. Using the techniques described in detail above above, speed feedback is provided by an accelerometer, e.g. mounted to the cannula hand piece or otherwise integrated with the surgical probe. The inner speed vs. power loop acts to limit laser power during instantaneous hand piece dwell (motion stoppage) thereby providing a convenient method to inhibit the laser when the hand piece stops moving, such that a more precise tissue temperature measurement may be made by the cannula thermister. Additionally, the speed vs. power or inner control loop prevents very rapid buildup of localized tissue temperature proximal to the fiber tip which could otherwise occur during a dwell period.

In some embodiments, this technique also allows flexibility in the placement of the thermister (relative to the tip and distance to heated tissue), and further reduces the fast time constant thermister requirement. In essence the power vs. speed loop controls very rapid tissue temperature increases (e.g. due to probe dwell), while the thermister more precisely controls the average tissue temperature increases which occur during the treatment process. In some embodiments, the thermister/thermocouple may be triggered to take a temperature measurement when the accelerometer data indicates that the handpiece is moving sufficiently slowly compared to the time constant of the thermister/thermocouple to allow for an accurate measurement.

The adjustable temperature command may by selected based on the type of procedure being performed (skin tightening vs deep lipo disruption), or it may be selected based on the body location being treated (neck/face vs abdomen).

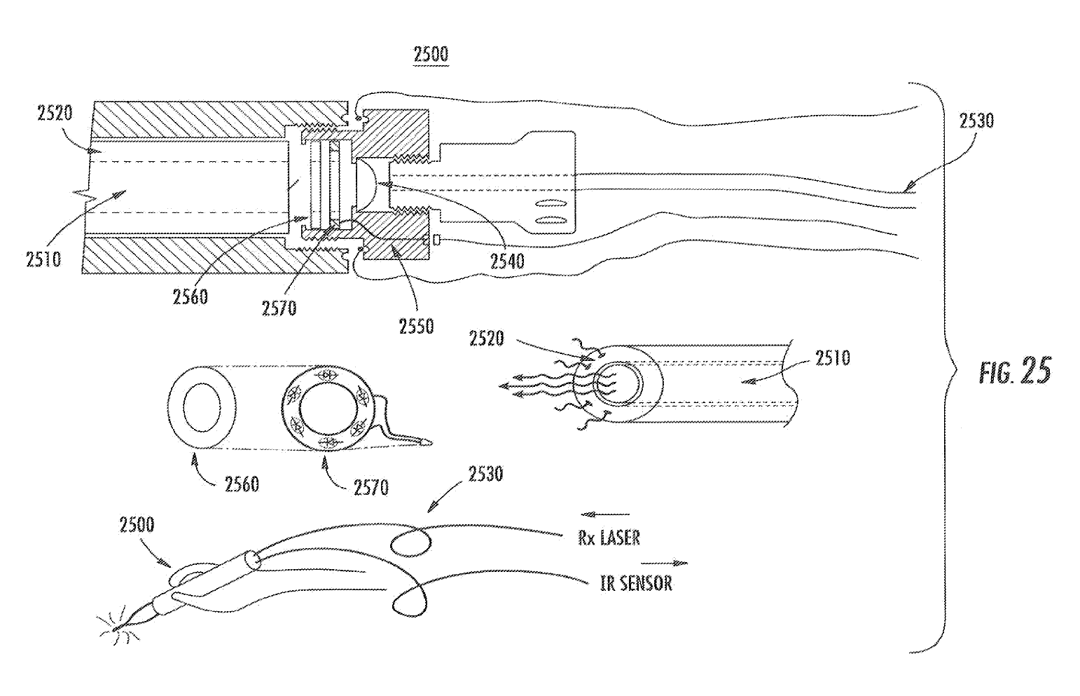

In some embodiments, handpiece position information derived from the accelerometer outputs may be combined with temperature information from the temperature sensor to provide, for example, a temperature map (e.g. a 2D or 3D map) of the treatment area. For example, referring to FIG. 23 A temporary 2D temperature map can be created from the combined data of the accelerometer and the temperature within tissue along a cannula reciprocal stroke path along a given surgical track. This is based on the fact that the reciprocal axis of the handpiece may be fixed in space for several seconds, or strokes. For example, in the embodiment shown, 1 sec/stroke a typical cycle, before a new surgical track is selected. During each one second one second, the temperature can be sampled more than 10 times and the information of probe position and temperature linked (t=0-3 s below) as shown in plots 2301. In typical applications, the information will be too transient and perhaps noisy to be useful to the clinician, but a running average of at least three stroke cycles will create a coarse time/temperature map 2302 of the temperature profile within the current surgical track. In the example shown, a quick glance by the clinician would indicate more accumulated energy/temperature 2303 near the right, incision side of the surgical track.

The change in direction of the handpiece can be sampled since the speed goes to zero. This concept works if the strokes only stop on the extreme ends and not within the stroke.

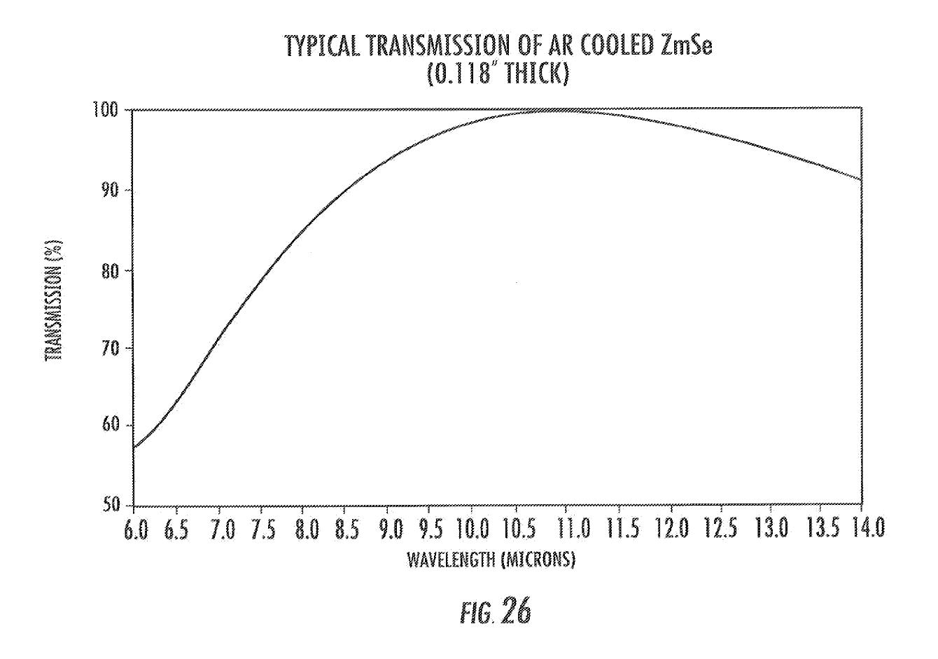

In some embodiments, the thermister or thermocouple may be replaced by other types of temperature sensors. For example, FIG. 24 shows an embodiment of a surgical laser waveguide 2400 which incorporates IR temperature sensing of tissue adjacent to the treatment waveguide/fiber tip 2410. An IR waveguide 2420 (e.g. a ZnSe IR fiber) is bundled with the surgical waveguide 2430 in an over-jacket 2440. In the example shown, a two sensor IR photodetector assembly 2450 is located in the hand piece 2460 adjacent to the treatment beam focus assembly 2470. Portions of light from the IR waveguide at two distinct wavelengths are separated and directed respectively to the two IR sensors using, for example, a dichroic beamsplitter 2480. Signals from the detectors are compared differentially to increase sensitivity and reject errors due to the “sense waveguide” transmission variables or characteristics.

The signals from the IR sensors are processed to obtain temperature information about the tissue under treatment. IR temperature monitoring provides tissue temperature feedback to the laser (which would adjust energy deposition based on observed tissue temperatures. In various embodiments, this could include a simple maximum temperature safety limit, or feedback could allow closed loop temperature control of tissues. In either case the laser takes feedback from the IR sensor and then adjusts laser output power (closed loop) to achieve the selected tissue temperature.

In some embodiments, the surgical waveguide itself can collect IR light from the treatment area during treatment to provide IR tissue temperature sensing. However, for some applications, such a waveguide or fiber would be required to pass high energy lasers in the 532 to 1550 nm wavelengths (treatment wavelengths) and also IR wavelengths of 3-14 μm, e.g. 3-5 μm or 8-12 μm (for temperature sensing and feedback). In some embodiments, this may be an unwanted requirement. FIG. 25 shows an example of a device 2500 which avoids this requirement by employing a dual fiber approach. As with the systems described above, light at a treatment wavelength is delivered via a waveguide 2510 (e.g. a stiffened fiber) suitable for surgical use without a cannula. The treatment waveguide 2510 is surrounded by and coaxial with an IR waveguide 2520 (.e.g. a ZnSe cylinder or tube). As described above, the treatment waveguide 2510 is coupled to a treatment fiber 2530 which delivers light from a treatment source. The coupling is accomplished using a focus assembly 2540 in a connector 2550 connected to the back of a hand piece. As shown, the connector also includes and IR pass filter ring 2560 (to filter out stray treatment light) and IR detector ring 2570 (e.g., an annular array of IR photodetectors), aligned with the IR waveguide tube 2520. The IR sensor ring produces electrical signals in response to incident IR light. These signals are passed to a processor, which operates to determine tissue temperature information and provide feedback to the treatment laser, as described above.

As described above, in various embodiments, IR light from a treatment area is propagated to an IR detector assembly via optics suitable for in vivo temperature monitoring. These optics may include, for example, coated ZnSe or Germanium rods or tubes, or certain IR transmissive plastics or even photonic waveguides (the IR transmission characteristics of AR coated ZnSe are shown in FIG. 26). Although several examples of IR optics are presented, it is to be understood that other suitable materials, geometries, and configurations may be used.

The temperature information acquired using the above described IR sensing techniques may be used in place of the thermister/thermocouple derived information in any of the techniques described above.

In some embodiments, a surgical probe is disclosed with a temperature sensor attached to cannula tip for purpose of measuring cannula temperature and shutting down laser should cannula become overheated. In various embodiments, the temperature sensor may include a negative temperature coefficient NTC or positive temperature coefficient PTC thermister or even IR photodetectors.

Some embodiments employ control method or algorithm where a temperature feedback signal from a temperature sensor is used to adjust laser output power by means of an error amplifier and compensation circuits.

Some embodiments employ a method or control algorithm that limits the temperature measured at the cannula tip for purpose of limiting laser output based on combined tissue and cannula tip temperature rise.

Some embodiments employ a method or control algorithm that, based on the temperature measured at a cannula tip of a laser surgical probe, limits laser output based on combined tissue and cannula tip temperature rise.

Some embodiments employ a method or control algorithm which adjusts the relative power of independent wavelengths of a multiplexed laser treatment pulse to effect a change in tissue temperature rise or treatment area to improve the homogenous deposition of energy and also temperature rise. Since penetrating depths vary for different laser wavelengths, simply adjusting the ratio of composite wavelengths adjusts the dimensions of the treatment space or treatment area.

Tissue Type Discrimination

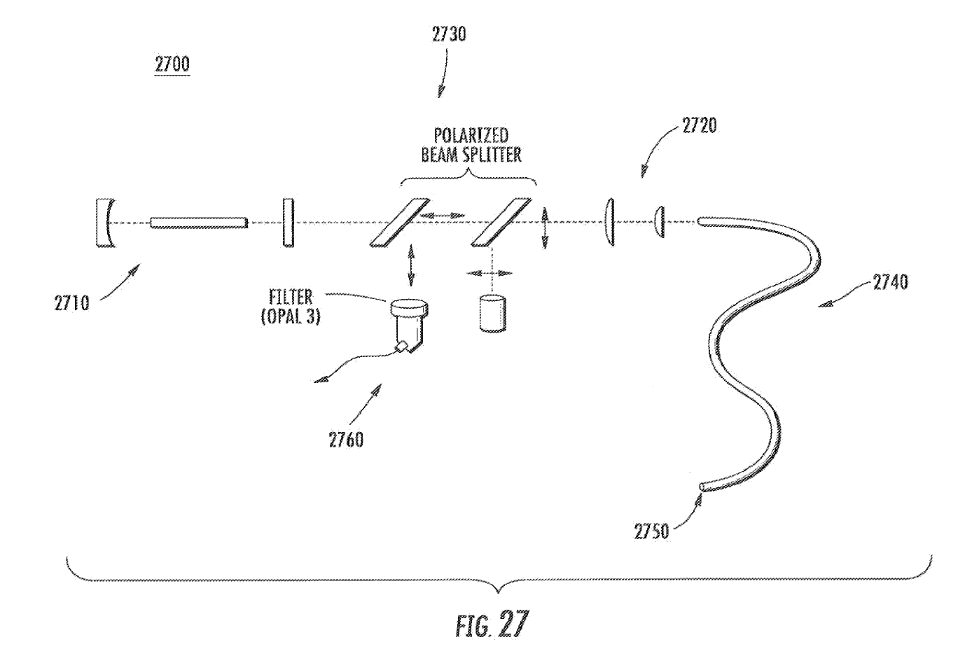

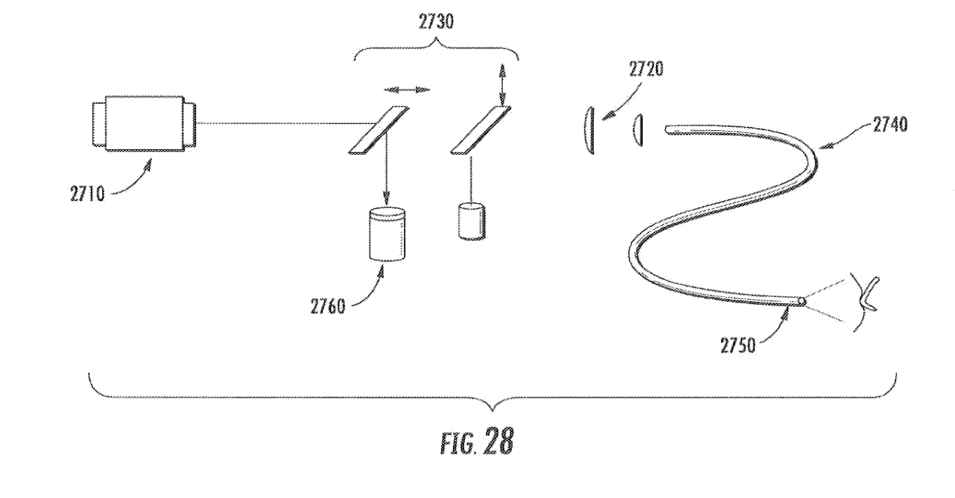

An exemplary probe beam injector 2700 with reflectivity and remittance color sensor is shown in FIGS. 27 and 28. A tissue treatment beam (in this example, with a wavelength of 1064 nm) is propagated from the output coupler (OC) of a treatment beam resonator cavity to a focus assembly 2720 via a polarized beam splitter 2730. The polarizer/beamsplitters are transparent to the 1064 nm treatment beam yet act as polarizers to one or more probe/doping beams. Accordingly, the probe beam sources at one or more wavelengths are coupled in to the path of the treatment beam, directed to the focus assembly 2720, propagated down a fiber 2740 or waveguide to an output tip 2750, and directed to tissue of interest. Similarly, reflected/remitted probe light from the tissue is collected and propagates back along the fiber 2740 or waveguide from the output tip 2750 and back through the focus assembly 2720 and is separated out from the path of the treatment beam and directed to one or more color photodetectors 2760. The photodetectors may include filters for filtering out stray treatment light and/or to distinguish between multiple probe light wavelengths (i.e. colors). Signals from the photodetectors (e.g. color and intensity), are processed, e.g. as described below, to characterize tissue and determine treatment (e.g., treatment beam intensity, pulse duration, etc.). For example, in laser lipolysis applications, if hidden vascular tissue, or other tissue unsuited for treatment is identified, the treatment laser is directed not to fire.

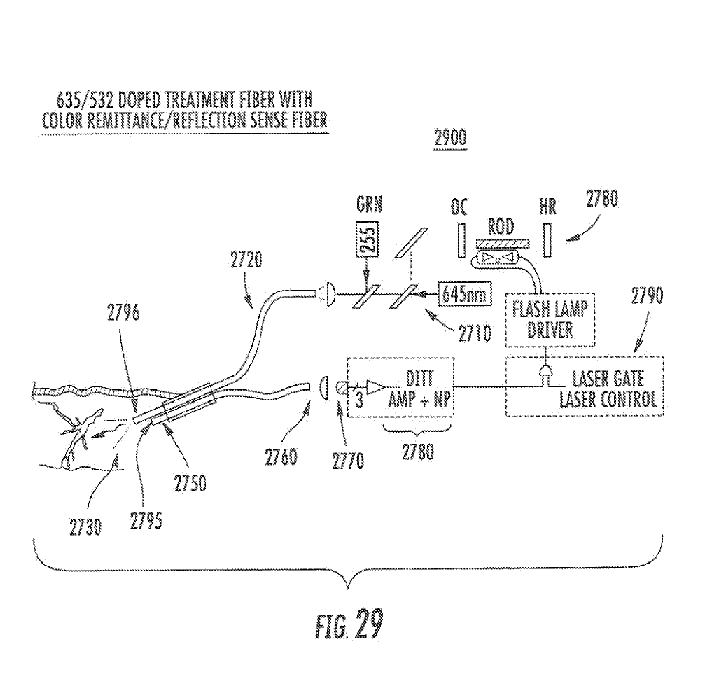

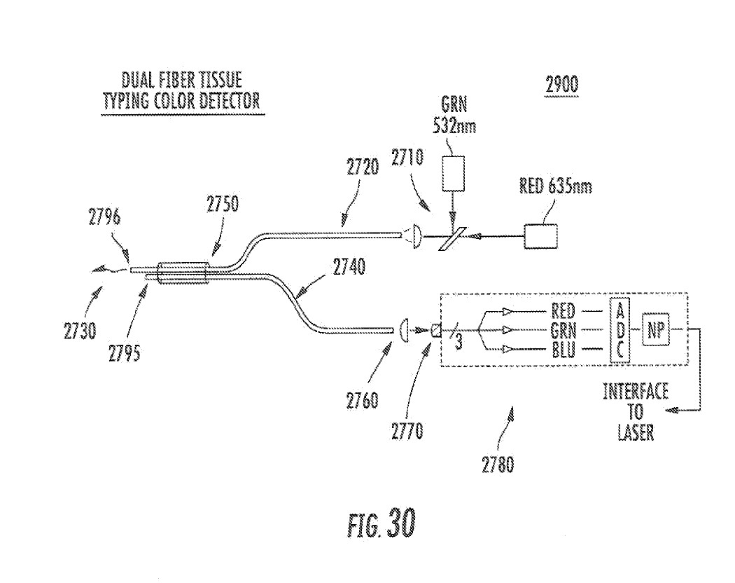

FIGS. 29 and 30 show examples of laser systems with tissue type determination featuring dual waveguides. As in the system described above, doping/probe light at multiple wavelengths/colors (as shown, 532 nm green and 635 nm red light) are coupled into the path of a treatment beam (e.g. using dichroic elements 2710 such as mirrors and/or beam combiners/splitters), and propagated down a treatment waveguide or fiber 2720 to a treatment area 2730. However, unlike the systems above, a second “sense” waveguide or fiber 2740 is included with the treatment fiber, e.g. in a cannula 2750 or catheter inserted into the patient. The sense fiber 2740 collects reflected/remitted light from tissue of interest, and propagates it back to a focus assembly 2760 and on to a color photodetector 2770 (e.g. an RGB photodetector). As with the systems above, signals from the photodetector are processed using processing electronics 2780 (e.g. differential amplifiers, analog to digital converters, microprocessors, etc, see below) for tissue determination. The results of the tissue determination are fed back to the treatments laser source 2780 (or laser source controller 2790) to control (e.g. provide or halt) treatment based on the determined tissue type. In some embodiments, the sense fiber tip 2795 can be offset from the treatment fiber tip 2796, as shown.

In various embodiments, visible or invisible wavelengths can be used for tissue type discrimination. (As mentioned above, in some embodiments the diagnostic and treatment beams are a single beam.) In some embodiments, at least 2 diagnostic wavelengths are used, although more wavelengths will improve precision and resolution. For example, aim-beam style low power visible lasers (e.g. lasers with power outputs in the range of about 1-50 mW) are readily available, low cost, and suitable for discrimination of the major tissues of interest common to laser lipolysis. For example human fat is yellow, fascia is white, and skin contains large amounts of darker pigments including red, etc. In some embodiments, the diagnostic “doping” or probe beams may be continuous wave (CW). In some embodiments, a time multi-plexed or pulsed combination of different wavelengths may also be used.

In some embodiments, it is possible to build a tissue type determination system based on a single wavelength diagnostic beam. The single wavelength is chosen so that there is a large difference in the absorption coefficient of the targeted lipids and the all the other tissues that are not targeted. However, such system heavily relies on a predetermined backscatter coupling efficiency. That is the total efficiency of delivering the diagnostic beam to the tissue in front of the tip, collecting the backscattered signal, and delivering the backscattered signal to a sensor in the laser system. Any changes in the fiber delivery system (like fiber tip contamination) would change the backscatter coupling efficiency and decrease the reliability of a single wavelength diagnostic system.

The reliability of the tissue type diagnostic can be greatly improved by using a multiple wavelength diagnostic beam. Increasing the number of wavelengths will increase the precision of the diagnostic system and allow it, for example, to distinguish between multiple chromophores.

As an example a two wavelength diagnostic system will be considered. In the example the system will be assumed to distinguish between fat (liposomes) and water. Most tissues in the body other than fat contain over 80% water. Therefore a diagnostic system that distinguishes between fat and water can be used to deliver energy when the fiber tip is pointing towards fat and not to deliver energy when the tip is pointing at any other tissue.

Although not intending to be bound by theory, the following example illustrates the operation of a two wavelength diagnostic system designed to determine the fat content in water environment. For each wavelength the signal propagates from the source to the detector. For wavelength 1 the source intensity is S₁. The total optical system and fiber transmission is T. The signal delivered at the end of the fiber is S₁T. Part of that signal is backscattered to the fiber with efficiency B while part of it is absorbed with efficiency A₁. The signal that arrives back at the fiber end is S₁TB(1−A₁). The backscattered signal is coupled to the fiber and transmitted to the detector with efficiency C, the detector has efficiency D₁. The signal arriving at the detector is S₁TB(1−A₁)CD₁. It will be assumed that if the two diagnostic wavelengths are sufficiently close (300 nm in the IR) the backscattering efficiency B does not depend on the wavelength or the fat content f. Then the only fat content dependent parameter is the absorption efficiency A. If the diagnosed tissue has an unknown fat content f, the detected signals in the two detectors V₁ and V₂ for the two wavelengths can be written as

V₁=FS₁TB(1−A₁^{F})CD₁+(1−f)S₁TB(1−A₁^{W})CD₁

V₂=fS₂TB(1−A₂^{F})CD₂+(1−f)S₂TB(1−A₂^{W})CD₂

where the indices 1 and two indicate wavelengths and the superscripts F and W indicate fat and water. The two equations can be rewritten as

V₁=S₁TBCD₁(1−A₁^{W})+fS₁TBCD₁((1−A₁^{F})−(1−A₁^{W}))

V₂=S₂TBCD₂(1−A₂^{W})+fS₂TBCD₂((1−A₂^{F})−(1−A₂^{W})) (1)

The parameters independent of tissue absorption can be eliminated by system calibration—that is by measuring the diagnostic signals V_{1c} and V_{2c} from a known sample with no fat content (f=0). The expressions for the calibration measurements are

V_{1c}=S₁TBCD₁(1−A₁^{W})

V_{2c}=S₂TBCD₂(1−A₁^{W})

The ratio of the two calibration measurements R_{c} can be defined as

$${R}_{c} = \frac{{V}_{2 c}}{{V}_{1 c}} = \frac{{S}_{2} {TBCD}_{2} ( 1 - {A}_{2}^{W} )}{{S}_{1} {TBCD}_{1} ( 1 - {A}_{1}^{W} )}$$

The calibration ratio may be obtained from a calibration tissue phantom before the laser lypolisys procedure begins and stored in the diagnostic system computer to be used in the real time tissue determination. During the laser treatment the diagnostic system runs the tissue determination procedure interspersed between the treatment pulses (or in parallel with a CW treatment beam) while the operator moves the treatment tip. The real time diagnostic signals V_{1d} and V_{2d} can be expressed from (1)

V_{1d}=S₁TBCD₁(1−A₁^{W})+fS₁TBCD₁(A₁^{W}−A₁^{F})

V_{2d}=S₂TBCD₂(1−A₂^{W})+fS₂TBCD₂(A₂^{W}−A₂^{F})

Based on the calibration measurement the last expression can be rewritten as

$${V}_{1 d} = {S}_{1} {TBCD}_{1} ( 1 - {A}_{1}^{W} ) + {fS}_{1} {TBCD}_{1} ( {A}_{1}^{W} - {A}_{1}^{F} )$$

$${V}_{2 d} = {R}_{c} {S}_{1} {TBCD}_{1} ( 1 - {A}_{1}^{W} ) + {fR}_{c} {S}_{1} {TBCD}_{1} \frac{( 1 - {A}_{1}^{W} )}{( 1 - {A}_{2}^{W} )} ( {A}_{2}^{W} - {A}_{2}^{F} ) .$$

The product S₁TBCD₁ can be expressed from the first equation and substituted in the second

$${S}_{1} {TBCD}_{1} = \frac{{V}_{1 d}}{( 1 - {A}_{1}^{W} ) + f ( {A}_{1}^{W} - {A}_{1}^{F} )}$$

$${V}_{2 d} = {R}_{c} \frac{{V}_{1 d}}{( 1 - {A}_{1}^{W} ) + {f ( {A}_{1}^{W} - {A}_{1}^{F} )}_{1}} ( 1 - {A}_{1}^{W} ) + {fR}_{c} \frac{{V}_{1 d}}{( 1 - {A}_{1}^{W} ) + f ( {A}_{1}^{W} - {A}_{1}^{F} )} \frac{( 1 - {A}_{1}^{W} )}{( 1 - {A}_{2}^{W} )} ( {A}_{2}^{W} - {A}_{2}^{F} )$$

The ratio of the two diagnostic measurements R_{d} can be defined as

$$\begin{matrix}{R}_{d} = \frac{{V}_{2 d}}{{V}_{1 d}} \\ = {R}_{c} \frac{( 1 - {A}_{1}^{W} )}{( 1 - {A}_{1}^{W} ) + {f ( {A}_{1}^{W} - {A}_{1}^{F} )}_{1}} + \\ {fR}_{c} \frac{( 1 - {A}_{1}^{W} )}{( 1 - {A}_{1}^{W} ) + f ( {A}_{1}^{W} - {A}_{1}^{F} )} \frac{( {A}_{2}^{W} - {A}_{2}^{F} )}{( 1 - {A}_{2}^{W} )}\end{matrix}$$

The last expression can be used to express the unknown fat content fraction

$$\begin{matrix}f = \frac{( {R}_{c} - {R}_{d} ) ( 1 - {A}_{1}^{W} )}{{R}_{d} ( {A}_{1}^{W} - {A}_{1}^{F} ) - {R}_{c} \frac{( 1 - {A}_{1}^{W} )}{( 1 - {A}_{2}^{W} )} ( {A}_{2}^{W} - {A}_{2}^{F} )} \\ = \frac{( {R}_{c} - {R}_{d} )}{{R}_{d} \frac{( {A}_{1}^{W} - {A}_{1}^{F} )}{( 1 - {A}_{1}^{W} )} - {R}_{c} \frac{( {A}_{2}^{W} - {A}_{2}^{F} )}{( 1 - {A}_{2}^{W} )}}\end{matrix} \qquad ( 2 )$$

The calculated tissue fat content f can be used by the tissue determination system based on a threshold value (for example when f>80%) to determine if the laser should be fired or not.

The expression for the tissue fat content (2) emphasizes the importance of choosing at least one wavelength so that there will be a large difference in the absorbed fractions in fat and water and at least one of the difference terms in the denominator will be large. One such wavelength region is 1300 to 1500 nm. A possible choice for large absorption difference wavelength is 1440 nm. The form of expression (2) would be simplified if the other wavelength is chosen so that the absorbed fractions in fat and water are nearly the same. Such wavelengths are for example around 1190, 1230, 1690 and 1730 nm. If one of the wavelengths (wavelength 1) is chosen so that the absorbed fractions in fat and water are nearly the same, the expression (2) for the fat content f becomes a linear function of the ratio of the two diagnostic measurements R_{d}.

$$f = {R}_{d} \frac{( 1 - {A}_{2}^{W} )}{{R}_{c} ( {A}_{2}^{W} - {A}_{2}^{F} )} - \frac{( 1 - {A}_{2}^{W} )}{( {A}_{2}^{W} - {A}_{2}^{F} )} \qquad ( 3 )$$

The expression (3) can be simplified further if the absorbed fraction in fat is neglected in comparison to the much larger absorbed fraction in water

$$f = {R}_{d} \frac{( 1 - {A}_{2}^{W} )}{{R}_{c} {A}_{2}^{W}} - \frac{( 1 - {A}_{2}^{W} )}{{A}_{2}^{W}} \qquad ( 4 )$$

Expression (4) can be rearranged to express the expected ratio of the diagnostic and calibration ratios (R_{d} and R_{c}) as a function of the fat content f

$${r}_{t} = \frac{{R}_{d}}{{R}_{c}} = \frac{( 1 - {A}_{2}^{W} + {fA}_{2}^{W} )}{( 1 - {A}_{2}^{W} )} \qquad ( 5 )$$

where rₜ can be interpreted as a tissue type ratio. It is clear from equation (5) that for very low fat content f≈0, the diagnostic ratio is equal to the calibration ratio and the tissue type ratio r_{.t}≈1. As the fat content increases (and for wavelength 2 fat having much lower absorption than water), the tissue type ratio grows.

In some embodiments, a diagnostic system a threshold tissue type ratio may be predetermined so that if the tissue type ratio exceeds the threshold, the sampled tissue in front of the tip of the delivery fiber will be considered to be fat. The threshold tissue type ratio can be calculated, for example, using equation (5) and absorbed fraction in water at wavelength 2. In some embodiments, the threshold tissue type ratio can be established by experimental measurements in excised tissue fat from fat reduction surgery.

In some embodiments, the operation of the tissue type determination can be greatly simplified with some loss of precision by a specific choice of diagnostic wavelengths. One such choice is when wavelength 1 is chosen so that water and fat have the same absorption, for example around 1230 nm. Then wavelength 2 is chosen so that water has nearly the same absorption and fat has a much lower absorption A_{1.}^{F=}A_{1.}^{W}=A^{W}≈A₂^{w}>>A₂^{F}. For example wavelength 2 can be chosen around 1290 nm. Other possible combinations of wavelengths 1 and 2 can be 930 nm and 1070 nm, 1730 nm and 1630 nm, 2320 nm and 2100 nm. For these wavelength choices the expressions (1) for the diagnostic signals at the two wavelengths simplify to

V₁=S₁TBCD(1−A^{W})

V₂=S₂TBCD₂(1=A^{W})+fS₂TBCD₂A^{W}

The source intensity is S₁ and S₂ and the detector has efficiencies D₁ and D₂ can be adjusted to be the same (for example using electronics). Then the diagnostic ratio of the two signals reduces to

$${ρ}_{d} = \frac{{V}_{2}}{{V}_{1}} = \frac{1 - {A}^{W} + {fA}^{W}}{1 - {A}^{W}}$$

Then for very low fat content the diagnostic ratio is around 1 and it grows with increasing fat content. A threshold tissue type ratio can be established either by calculations or by experimental measurements in excised tissue fat from fat reduction surgery.

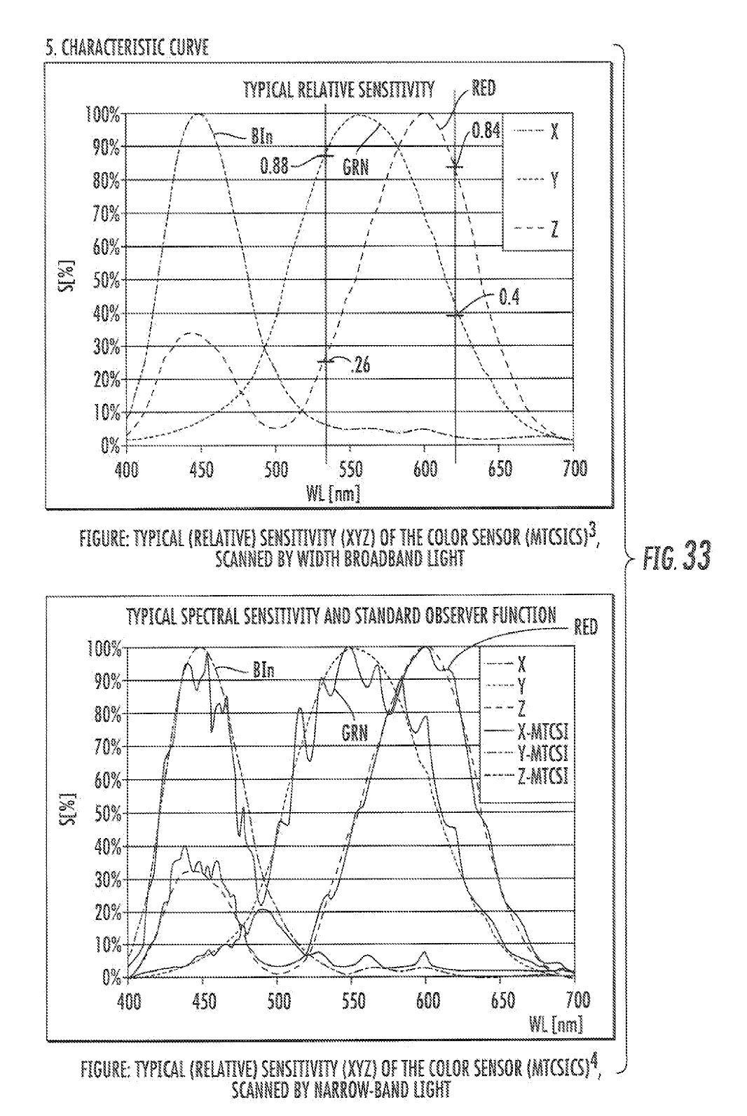

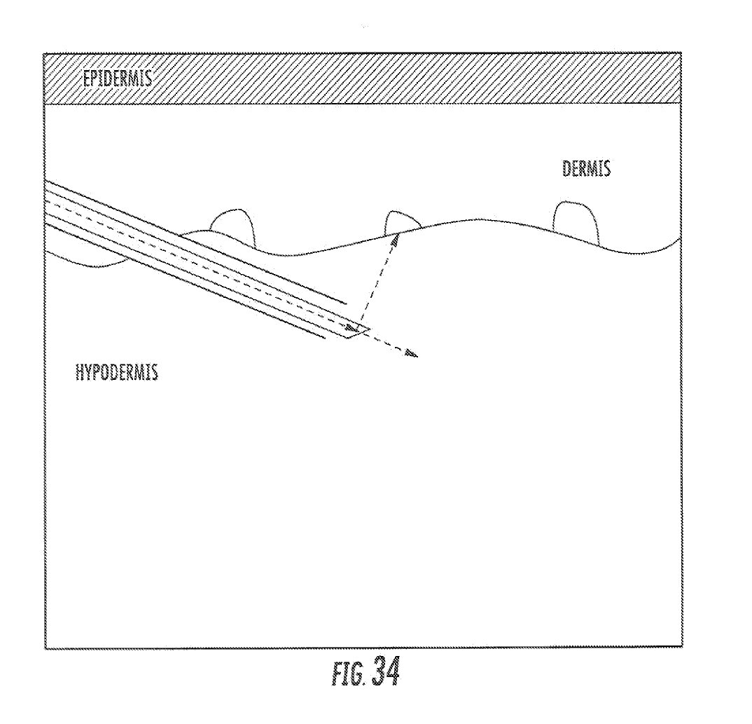

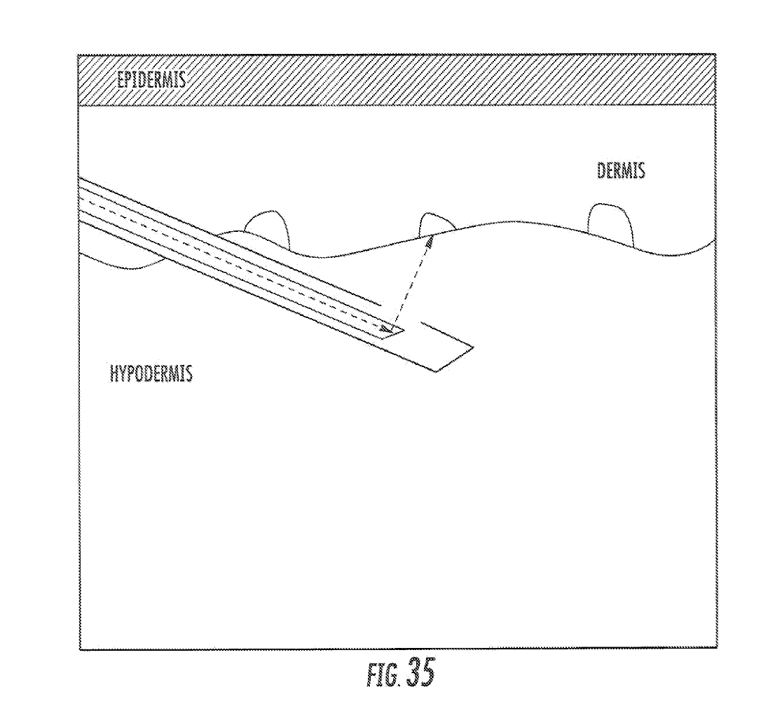

FIG. 31 shows an exemplary circuit 3100 for use in processing signals detected by a color photodetector. As shown, an MTCSiCO Integral True Color Sensor type TO39 is used as the color photodetector. The TO39 includes three photodiodes which each produce photocurrents in response to light at a different frequency (the spectral response characteristics of the respective photodiodes are shown in FIG. 33). An amplifying circuit features a three op amp package OPA491, configured to convert the respective photocurrents from the TO39 into voltages. Variable resistors are provided to selectively adjust the response of the amplifying circuit to each of the three photocurrent “channels.” As described above, such control of detector response efficiencies can be used to simplify tissue determination

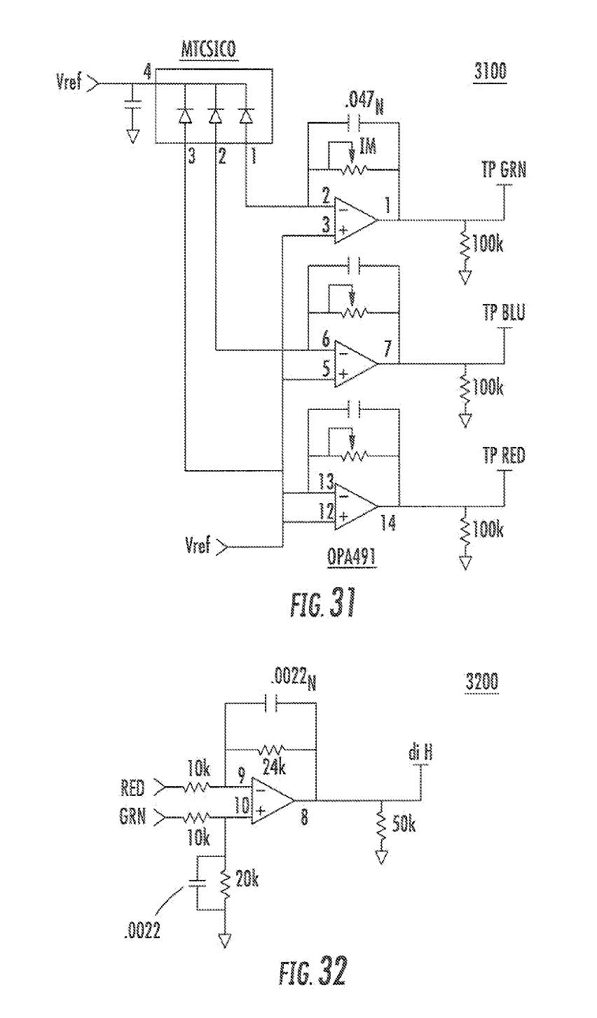

FIG. 32 also shows an example of a differential amplifier 3200 for use in tissue type determination using the techniques described above. The differential amplifier produces a voltage difference across its output terminals which is representative of the difference in photocurrent measured by each of two photodiodes corresponding to different detected wavelengths.

It is to be understood that the light collected for tissue type analysis may include, for example, reflected probe/doping light, scattered or refracted probe/doping light, remitted light, stimulated fluorescence or phosphorescence, or any other light indicative of tissue type.

Embodiments of the present invention described herein are directed to devices and methods that can be used in a surgical procedures. One example of the surgical procedures is lipolysis.

While this invention has been particularly shown and described with references to example embodiments thereof, it will be understood by those skilled in the art that various changes in form and details may be made therein without departing from the scope of the invention encompassed by the appended claims.

One or more or any part thereof of the tissue determination techniques described above can be implemented in computer hardware or software, or a combination of both. The methods can be implemented in computer programs using standard programming techniques following the method and figures described herein. Program code is applied to input data to perform the functions described herein and generate output information. The output information is applied to one or more output devices such as a display monitor. Each program may be implemented in a high level procedural or object oriented programming language to communicate with a computer system. However, the programs can be implemented in assembly or machine language, if desired. In any case, the language can be a compiled or interpreted language. Moreover, the program can run on dedicated integrated circuits preprogrammed for that purpose.

Each such computer program is preferably stored on a storage medium or device (e.g., ROM or magnetic diskette) readable by a general or special purpose programmable computer, for configuring and operating the computer when the storage media or device is read by the computer to perform the procedures described herein. The computer program can also reside in cache or main memory during program execution. The analysis method can also be implemented as a computer-readable storage medium, configured with a computer program, where the storage medium so configured causes a computer to operate in a specific and predefined manner to perform the functions described herein.

### Example 1

### Invasive Treatment of Cellulite

According to various studies, cellulite concerns 85-98% of post pubertal females. The term “cellulite” describes the “orange peel” syndrome or quilted appearance in areas with subcutaneous fat. This condition is most commonly observed on the thighs, the arms and the abdomen. Numerous therapies both non-invasive and invasive have been suggested, such as mesotherapy, treatment with an energy source (such as a laser or radiofrequency device) or a combination of both, and subcision in the subdermal layer. However, none of them has been proven as a permanent cure for cellulite.

A distinctive structural feature of cellulite is the presence of subcutaneous fat herniations into the reticular and papillary dermis. A common goal in most non-invasive treatments is to eliminate the fat intruded in the dermis and alter connective tissue which creates herniations of fat at the dermal-hypodermal interface. Several studies have shown that mesotherapy can temporarily reduce the fat herniation in the dermis and flatten the dermal-hypodermal interface. However, the adipocytes will re-grow into dermal region and the improvement of cellulite only last for couple months.

Subcision is an invasive treatment for cellulite. It is performed using a tri-beveled hypodermic needle inserted it through a puncture in the skin surface. The sharp edges of the needle are maneuvered under the cellulite skin in a repetitive back and forth movement. The idea is to break the connective tissue that has secured the fat-herniated skin to the underlying tissue. It frees up the skin surface from the underlying tissue and cause the skin to appear even and smooth. However, this treatment does not alter the fat pockets intruded in the dermis and the broken connective tissue will eventually reconnect in the same fashion. Therefore, cellulite appearance is not significantly improved. Thus, there remains a need in the field for cellulite treatment methods that result in long lasting improvement for cellulite.

A preferred invasive approach to cellulite treatment delivers energy directly to the dermal-hypodermal interface. Since the energy does not traverse the upper layers of the skin, the possibility exists for an aggressive treatment that can: 1) break the connective tissue to free up the skin surface in a manner similar to subcision; 2) thermally denature the intruded adipocytes in the dermis; and 3) induce significant collagen growth and even subdermal scar formation at dermal-hypodermal junction to tighten the skin. This approach makes possible significant improvement on cellulite over the current therapies.

A preferred device to perform the procedure above consists of a number of components that include an energy source such as a laser, a delivery system such as a “side-firing” optical fiber that can direct the light energy to its side, a means of locating and positioning the fiber underneath the interface between the dermis and hypodermis such as a cannula, and sensors to monitor the treatment process such as temperature and position sensors. In the case of a laser source and “side-firing” optical fiber delivery system the wavelength and laser intensity are chosen to control the extent of the exposure to the neighborhood of the fiber. This allows the practitioner to create regions of thermal damage in deep dermis and hypodermis.

An embodiment that allows the procedure above includes a laser source, a “side-firing” optical fiber, and a cannula to direct the fiber underneath the dermal-hypodermal junction. FIG. 34 shows an embodiment of the invention where an area of a patient is treated. An optical delivery device is inserted into the patient such that a light emitting portion of the device is proximal to an interface between the dermis of the skin and the underlying fascia of the patient, shown in FIG. 34 as the hypodermis. The hypodermis (also called the hypoderm, subcutaneous tissue, or superficial fascia) is the lowermost layer of the integument. Types of cells that are found in the hypodermis are fibroblasts, adipose cells, and macrophages. Therapeutic light is delivered from the light emitting portion of the delivery device to heat a target region located proximal to the interface to cause thermal damage in the target region without causing substantial thermal damage to dermal and epidermal tissue located above the target region. As shown in FIG. 34, the optical delivery device emits light in two perpendicular directions, disrupting target adipocyes within subcutaneous fat herniations, as well as remodeling collagen and cauterizing blood vessels.

FIG. 35 shows a similar device as FIG. 34, wherein in this embodiment the optical delivery device has a side firing optical fiber, which extends along a longitudinal axis from a first end to a second end, and delivers therapeutic light from the light emitting side portion of the delivery device. This device includes a cannula having a sharpened tip, facilitating movement of the device through the patient's tissues.

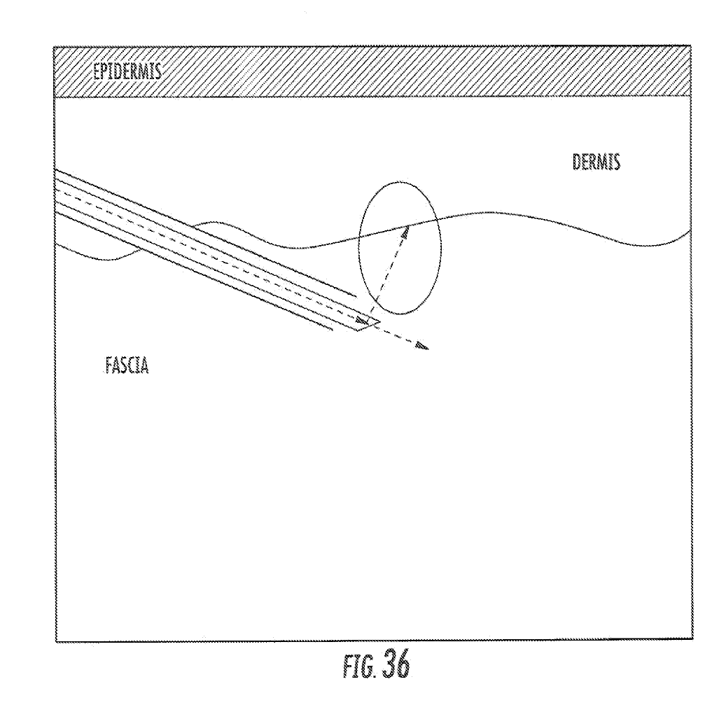

The laser can be one of any of a number of available sources whose radiation is strongly absorbed by either blood or tissue. The wavelength of operation of lasers meeting this requirement can be in the visible or infrared regions of the electromagnetic spectrum. One preferable laser source is a near infrared laser, more preferably one operating at a wavelength in the neighborhood of 1440 nm. This wavelength has been shown in both animal studies and abdominoplasty studies to yield high temperature gradient along the direction of energy deposition. This allows heating of the dermal-hypodermal interface above 50° C. while still keep the upper dermis and epidermis temperatures below 42° C. to avoid tissue damage proximal to the treatment site. FIG. 36 shows an embodiment of the invention wherein therapeutic light is delivered from the light emitting portion of the delivery device to heat a target region located proximal to the interface of the dermis and fascia. The heating of the target region is substantially localized to within a desired distance above and below the interface of these tissues. Heating of the target region proximal to the interface results in a temperature of about 50° C. or more in this target region, while the upper dermal and epidermal tissue located above the target region is maintained at a temperature of about 42° C. or less, disrupting adipocyes within subcutaneous fat herniations in the target region, as well as remodeling collagen and cauterizing blood vessels, without causing substantial thermal injury to the tissues outside of the target region.

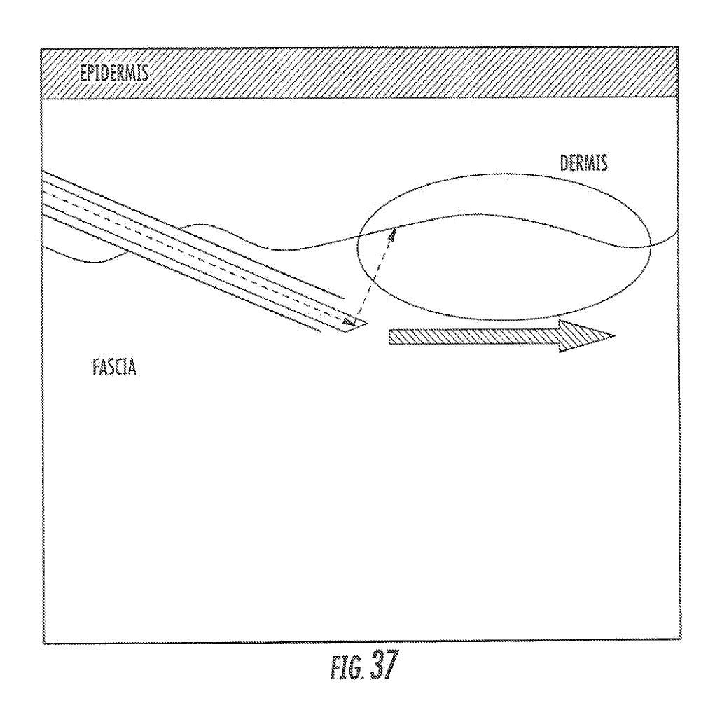

FIG. 37 shows a similar embodiment as FIG. 36, but further illustrates that the device is manipulated so the light emitting portion of the optical delivery device moves along the dermal interface while delivering the therapeutic light across an expanded target region.

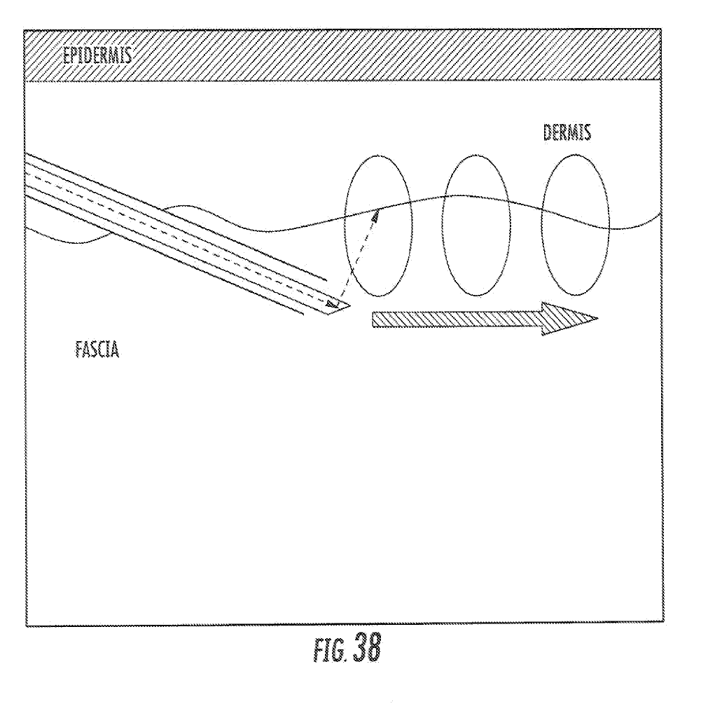

The laser intensity should be sufficient to heat the dermal-hypodermal junction above its normal temperature, preferably ten degrees or more. This will render the tissue nonviable and will result in its replacement with new collagen over the following weeks. At 1440 nm with a 0.6 mm diameter “side-firing” fiber an intensity in the range 4 to 20 watts is preferable, more preferably about 8 watts. The laser pulse duration and repetition rate can vary over a very broad range from continuous wave to short high intensity pulses. At an operating wavelength of 1440 nm pulsed lasers are preferable since these have been shown to provides better hemostasis, a more preferable embodiment being a pulse duration on the order of 0.5 ms, and repetition rate on the order of 40 hz. FIG. 38 illustrates an embodiment wherein therapeutic light from the light emitting portion of the delivery device is generated as a series of light pulses. Exemplary pulse durations of about 0.1 ms to about 1.0 ms and more preferably about 0.5 ms are employed. A repetition rate of about 10 to about 100 Hz and more preferably about 40 Hz is used.

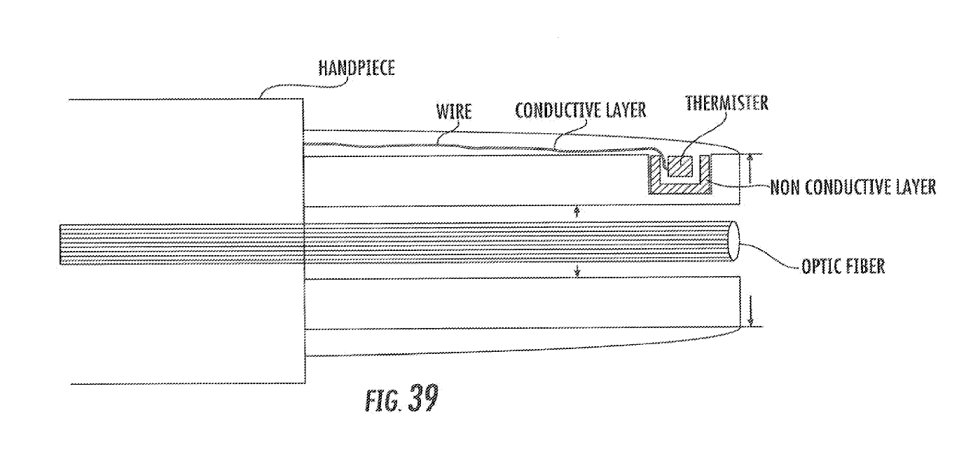

In addition the device is fitted with a thermal sensor such as a thermistor located near the distal end of the fiber. Beneficial additions to the embodiment above are motion sensors such as an accelerometer. Such an addition allows the intensity of the laser to be controlled resulting more uniform treatment regions. The addition of thermal and position sensors permits better control of the treatment environment and improves the safety of the procedure. FIG. 39 illustrates an embodiment of the invention wherein the delivery device includes a thermal sensor means. A thermister is incorporated into the delivery device and is offset from the proximal end of the device. The thermister is in communication with a thermally conductive layer on the outside of the cannula, which allow it to sense the temperature of the target region. It is thermally insulated from the optical fiber and the proximal tip of the device. The insulation prevents heating of the thermister from the beam and further limits heat effects from heated cellular debris at the device tip or matter that is aspirated from the surgical site.

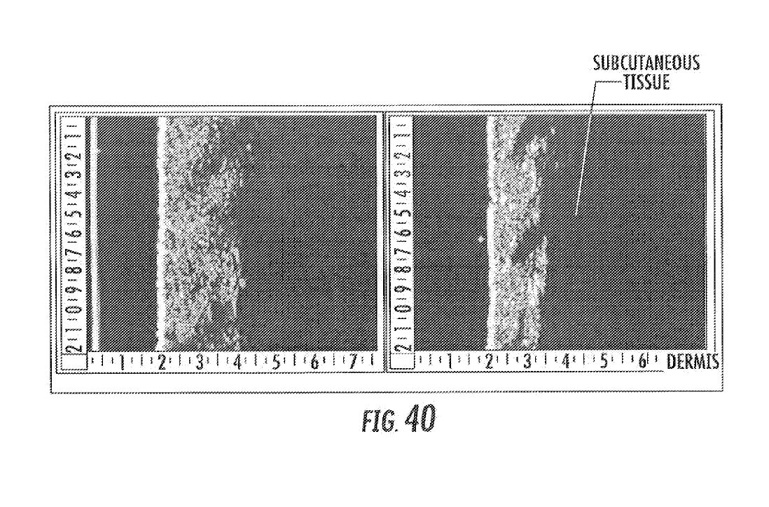

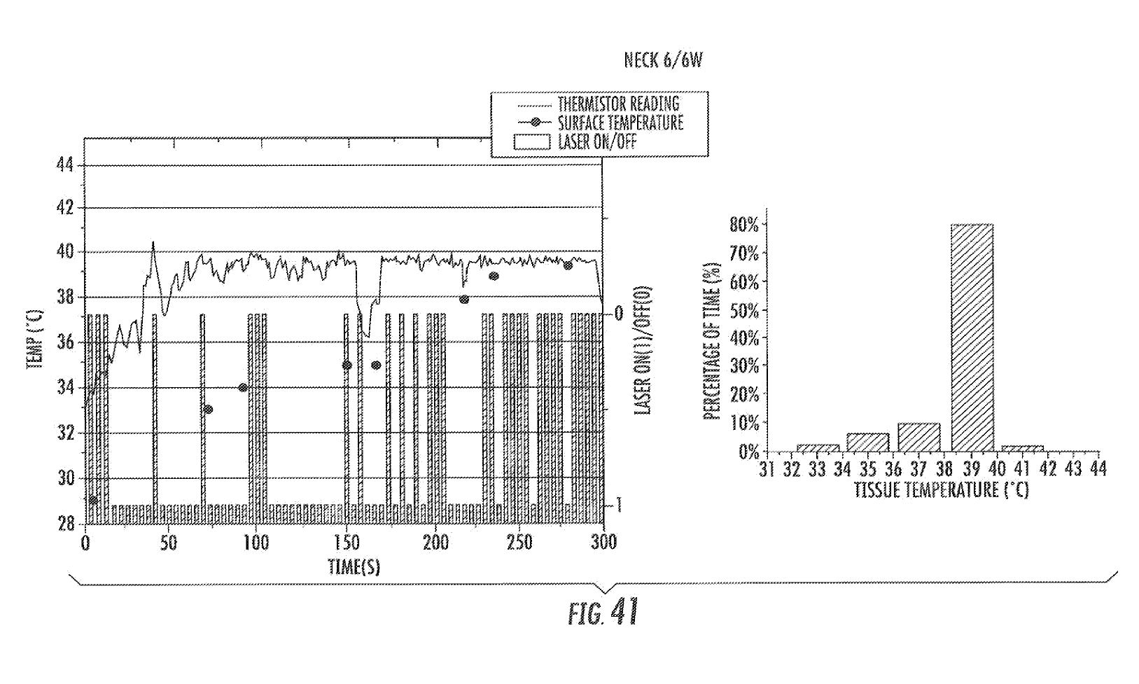

High-frequency ultrasound imaging post laser treatments have shown that the dermal-hypodermal interface was flattened, significant amount of new collagen were deposited under the dermal-hypodermal junction and the fat pockets in the dermal region were gradually replaced by fibrotic tissue. FIG. 40 shows a high frequency ultrasound image of skin. The right panel shows a baseline image on the thigh of a cellulite patient. The left panel shows the treatment site in the same patient one month after therapeutic laser treatment using a 1440 nm wavelength pulse laser with a side firing fiber. The “side-firing” optical fiber can be any of several available fibers which direct laser energy away from its axis. One preferable side-firing design is to redirect part of the laser energy to its side and leave the rest of the energy going forward along its axis. Such side-firing configuration thermally alters the septa underneath the skin while the redirected energy thermally denatures the dermal-hypodermal junction and promotes collagen growth in the dermis and herniated fat pockets.

The device above can be used in conjunction with current cellulite treatments such as mesotherapy or a combination of massage, laser and RF for further smoothing the skin surface and helping in directing new collagen growth during healing.

### Example 2

### Minimally Invasive Face Lift Systems

Anti-aging treatments using lasers and other energy sources range from very mild treatments such as low intensity LED treatments to more aggressive, ablative resurfacing methods. All these treatments result in some degree of skin improvement, not surprisingly the more aggressive treatments being the more efficacious. For the patient with significant laxity and desiring a greater improvement surgical intervention such as a face lifting procedure is the next step. These procedures are generally administered by plastic surgeons and involve extensive surgery and extended recovery. They are by nature costly and more amenable to complications during the recovery period. There is currently a need for an intermediate procedure that allows a controlled delivery of energy subdermally, one that is more aggressive and invasive than the common laser treatments but still less than a full face lift.

Disclosed herein are anti-aging treatment devices and procedures for a controlled subdermal delivery of energy. A common goal in most laser treatment is the stimulation of new collagen growth. In most cases this is achieved by exposing a region of skin to laser radiation. If properly chosen, the radiation will penetrate into the dermis, gently heat the underlying tissue and set in motion a response that will result in new collagen growth. Depending on the amount of new collagen, results can show significant improvement in skin appearance. These techniques are limited by the need to traverse the upper layers of skin which can often be damaged by surface application of laser energy.

In the case of a standard surgical face lift, the overlaying skin is first detached. The underlying fascia is surgically altered and the skin reattached. Here again one relies on the growth of new collagen to anchor the skin back onto the fascia and improve the skin appearance. An intermediate approach between surface application of laser energy and surgical facial detachment and ligation is to deliver energy directly to the interface between the skin and fascia. Since one is not traversing the upper layers, the possibility exists for an aggressive treatment that can induce significant collagen growth and even subdermal scar formation. If this procedure is performed over carefully chosen regions of the face it is possible to obtain significant improvement over the transdermal methods. In addition, if one relocates the skin during the healing process the result can be equivalent to mild lifting. The present device allows the practitioner to perform this intermediate procedure.

The device consists of a number of components that include an energy source such as a laser, a delivery system such as an optical fiber, a means of locating the fiber at the interface between the dermis and fascia such as a cannula, and preferably sensors to monitor the treatment process such as temperature and position/speed of the delivery device. In the case of a laser source and optical fiber delivery system the wavelength and laser intensity are chosen to control the extent of the energy delivered to the target region. This allows the practitioner to create regions of extensive new collagen growth and even scars that are located and oriented to enhance the appearance of the skin. In what follows further details of the proposed device are given using a laser and optical fiber delivery system as the preferred embodiment.

In a preferred embodiment, the procedure utilizes a laser source, an optical fiber, and a cannula to direct the fiber under the dermis and along the dermis fascia interface. In addition the device is fitted with a thermal sensor such as a thermistor located near the distal end of the fiber and position and motion sensors such an accelerometer. The laser can be one of any of a number of available sources whose radiation is strongly absorbed by either blood or tissue. The wavelength of operation of lasers meeting this requirement can be in the visible or infrared regions of the electromagnetic spectrum. One preferable laser source is a near infrared laser, more preferably one operating at a wavelength in the neighborhood of 1440 nm. This wave length has been shown in both animal studies and abdominoplasty studies to yield very localized (several fiber diameter) targeted regions of damage. In histological examinations, the passage of the fiber through the adipose tissue lying between the dermis and fascia was seen to result in a channel of damaged tissue. Adipose cells within this channel were subsequently cleared and replaced with fibrotic tissue. Exposures near the dermis interface resulted in even more intense collagen response. The laser intensity should be sufficient to heat the tissue more than six and preferably about ten degrees above its normal temperature. This will render the tissue non viable and will result in its necrosis over the following weeks. At 1440 nm with a 0.6 mm diameter delivery fiber an intensity in the range 4 to 20 watts is preferable, more preferable about 12 watts. The laser pulse duration and repetition rate can vary over a very broad range from continuous wave to short high intensity pulses. At an operating wavelength of 1440 nm pulsed lasers are preferable since these have been shown to provide better hemostasis, a more preferable embodiment being a pulse duration on the order of 0.5 ms, and repetition rate on the order of 40 hz. Beneficial additions to the embodiment described above are motion sensors such as an accelerometer. Such an addition allows the intensity of the laser to be controlled resulting more uniform treatment regions. The addition of thermal sensors also permits control of the treatment environment and improves the safety of the procedure.

In another embodiment, the device above can be used in conjunction with skin repositioning methods such as lifting threads or dressings to achieve an effect similar to a face lift procedure. The skin is maintained in a desired position while new fibrotic tissue develops under the skin. If sufficient new growth and mild scaring takes place the tissue will be held in place by this new fibrotic growth. In yet another embodiment, the handpiece (fiber and cannula) are slowly pulled or pushed under the skin to form a sub-dermal scar line. Since the speed is known (accelerometer) and laser power under direct control, precise dosimetry of Watts per linear centimeter traversed can be delivered. In this way, uniform scars can be created that are tuned to the desired end point temperature. One further development of this idea is to modulate the power along the scar line to form “barbs” or regions where the tissue damage and subsequent collagen remodeling effect an increased diameter. These barbs act as stays to anchor the scar line and hold the tissue more effectively in place. The regions of variable damage can also be generated with different wavelengths with different penetration depths.

### Example 3

### Minimally Invasive Isothermal Skin Therapy

In laser-based cosmetic surgery procedures, the endpoint temperature is critical to optimize tissue tightening, initiate collagen remodeling, and safely not exceed temperature maxima. For any source (laser, RF, ultrasound, microwave) the endpoint temperature is critical. Several clinical studies have demonstrated that the skin would become necrotic if temperatures went beyond approximately 47 C for a duration on the order of minutes. Also, hard scar tissue could be created if a large volume of subcutaneous tissue was heated over a critical temperature. Adding a temperature monitoring device to the delivery device or cannula is described above, but the following details the results of devices that have been put into clinical practice.

For laser-based procedures, the idea involves penetrating the fat layer plane through a minimum of one, or multiple incision sites, under the dermis with an optical fiber transmitting laser power. The laser power is designed to deliver sufficient power to disrupt fat cells and most of the energy is eventually converted to heat. Initially, this heat is localized to the immediate proximity of the fiber tip, but as the cannula is reciprocated through the tissue, the heat is distributed over a large area (˜20-200 cm2) and as time progresses, hot areas conduct to cooler ones and the temperature distribution becomes more even. This has been confirmed with thermal cameras focused on the surface of the skin: the thermal camera sees a relatively even surface temperature (±5° C.) even when the localized temperatures underneath can have deltas of 30° C. Using a thermistor as a temperature monitoring means incorporated into the device itself, one can regulate the deposition of laser energy to those areas under treatment below the set treatment temperature. Variables such as technique (speed, overlap), laser power, and wavelength can be brought under control since the endpoint, subdermal temperature, is kept constant. By doing so, the differential between surface temperature and deep tissue temperature is maintained and tissue damaging temperature maxima are avoided all together. The method of controlling the laser via the thermistor is simple: When the sensed temperature is above a user adjustable set point, the laser turns off, thus protecting the tissue and optimizing the result. For an ideal probe where the thermal response time of the probe was close to a millisecond, the laser would only deposit energy where the tissue was below the setpoint and soon an even temperature distribution would result, nearly independent of laser power, wavelength or technique. Practically, the speed of the cannula is approximately 10 cm/s and the response time 250 ms. Therefore, the cannula would have traveled 2.5 cm before the cannula would deliver a reading, clearly out of phase. To keep the overall ID to a minimum and keep the thermal response time short, the temperature monitoring device needs to be small. A thermistor was chosen due to its biocompatible components, good accuracy and stability, good operating range, ease of signal processing and small size. An optimized design has the thermistor insulated from the cannula and with good thermal contact to its surroundings. However, to minimize the ID of the cannula, the thermistor was inset into the wall of the cannula inside a machined slot. The cannula and thermistor combo were overcoated with a heatshrink covering to protect the thermistor from surgical wear cycling through fibrous tissue. These two construction elements slow the response of the thermistor (tau=250 ms). A design that uses an insulating layer between the cannula and the thermistor and a conductive, protective layer is preferred. Another advantage of the thermistor mounted to the cannula is that is can detect a fiber tip which has slipped into the cannula. Without the thermistor, the cannula would overheat, destroy the fiber and cause adverse tissue effects. The thermistor can detect the problem and automatically shut off the laser. If the fiber has retracted significantly within the cannula, well past the location of the temperature sensing element near the tip, the laser will briefly overheat the cannula and open circuit the connections to the thermistor also causing a fault event. However, despite these design drawbacks, if the response is averaged over the last few readings and the cannula motion remains vigorous, traversing back and forth at ˜10 cm/s across the surgical field, the result becomes more of an average of the tissue volume temperature. This has been demonstrated in a clinical setting using a datalogger to record the tissue volume temperatures and simultaneously monitoring the surface with a thermal camera. Despite the mismatch of sensor response time and the speed of the cannula, the temperature profiles are amazingly uniform, creating isothermal areas that span the entire treatment area. This effect is due in part to thermal diffusion but also due to the fat cell's ability to hold the heat for long periods, or thermal capacity. The current laser software allows the user to select a treatment temperature limit threshold above which the laser will not fire. The thermistor temperature feedback inhibits laser output for as long as the thermistor reads equal to or above the set-point. While the temperature control circuit fundamentally acts as a “bang-bang” control, the feedback signal has an adjustable running average applied to it such that the feedback signal response time can be varied from about 0.1 to 10 seconds. This filter setting allows the temperature controller to respond quickly to temperature changes (0.1 second) or slowly over the course of 10 seconds. An average of 1 second seems to offer the best control. As the proper dose to a certain area is approached, the laser will periodically stop firing, depositing less energy to that area and more to underexposed (lower temperature) areas. The average power will only be less than the laser power setting. If regulatory hurdles can be overcome, AUTO mode can operate the laser at full power initially (biggest temp differential between start and set point) and throttle back the power as the set point is reached, eventually cutting off the laser all together. The accelerometer still plays a role in any system employing this isothermal technique. For example, if the cannula stops and the laser is still generating high power, the tissue temperature at the tip of the fiber will increase rapidly. It will take an unacceptably long period for temperature rise to be detected by the thermistor which is adjacent to, but not coincident with, the fiber tip. It is therefore important that the fiber be moving for this system to work. It is also be possible to monitor and include the direction of the stroke. Due to the thermistor placement relative to the fiber tip, a slight temperature offset will occur. The highest offset temperatures are read as the heated tissue passes over the fiber tip (fiber pushing into tissue) and the lower offset temperatures as the thermistor pulls through the tissue. An added level of regulation could be applied by monitoring the direction and amplitude of the cannula movement, both within the capabilities of the accelerometer.

An embodiment is disclosed wherein the accelerometer feedback is used to dynamically set the time constant (tau) of the temperature feedback filter. The ideal time constant is directly proportionate to the cannula travel speed. The advantage of this approach is that normal surgical procedure stroke speed variations can be actively compensated such that the temperature controller has a constant filter tau vs stroke speed. In other words, we keep the thermistor phase lag constant as a function of cannula travel speed. This prevents the extreme out of phase behavior that could actually aggravate the evenness of the temperature deposition by the laser (eg. 180 degrees out of phase would actually inhibit the laser while in cool areas, and enable it in hot areas). Additionally, it is possible to manage the cannula direction versus temperature offset since the accelerometer speed feedback is bipolar.

The thermistor control helps not only avoid tissue necrosis due to over-heating, but also regulates the delivery of laser energy in a uniform way to achieve consistent treatment efficacy. FIG. 41 left panel illustrates the correlation of thermistor reading in tissue with skin surface temperature. The set treatment temperature (40 C in this case) was quickly reached while the skin surface temperature did not rise as much. The delivery of laser energy was continued with a thermistor regulation until the surface temperature endpoint was reached. FIG. 41 right panel counts the frequency at each temperature during the treatment. It showed that during 80% of the treatment time, tissue was heated to the set temperature of 39-40 C. This uniform subdermal heating helps physicians achieve consistent efficacy over the whole treatment area.

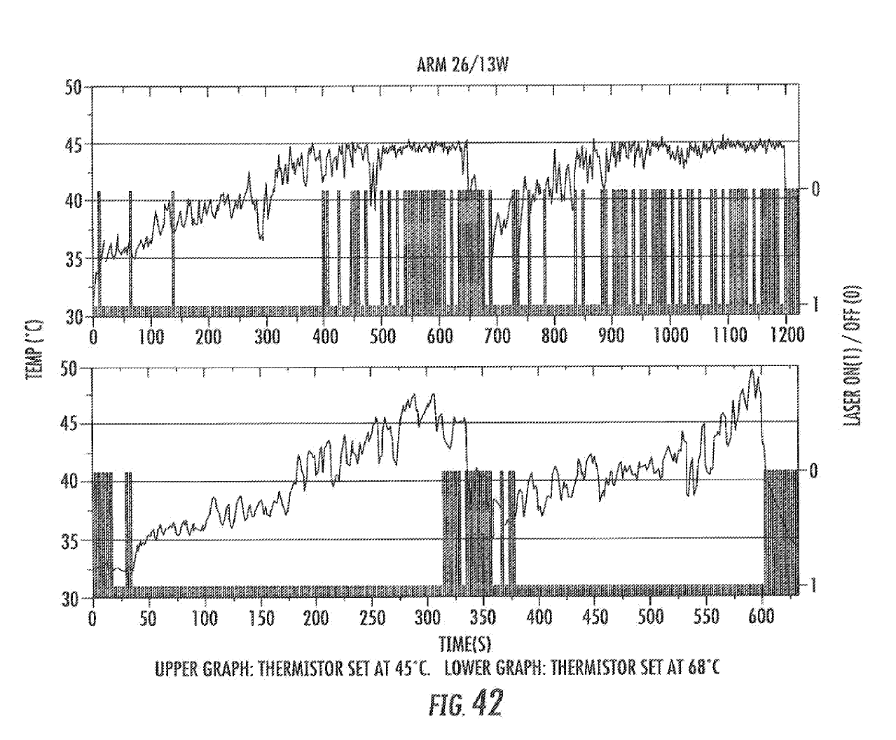

The following FIGs demonstrate the abilities of such a system. The two cases in FIG. 42 show the same procedure done at the same laser power, with and without the temperature feedback. In the upper panel, the thermistor feedback prevented any temperature rise within the tissue to rise above 45 C, the set point adjustable by the user. As a control, the lower panel shows the same procedure without such a control (A high set point of 68 C was chosen).

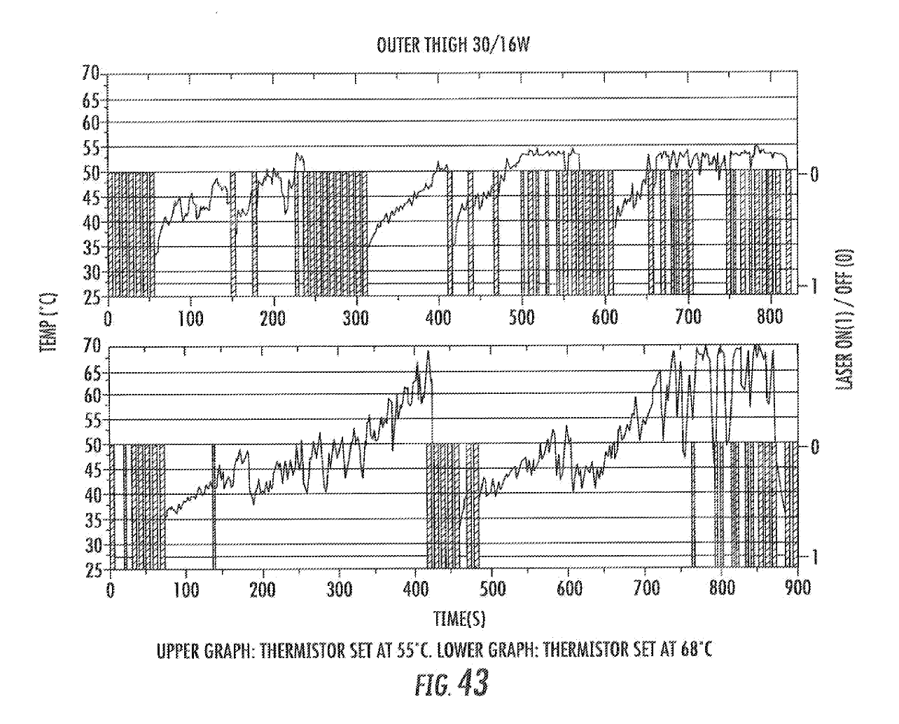

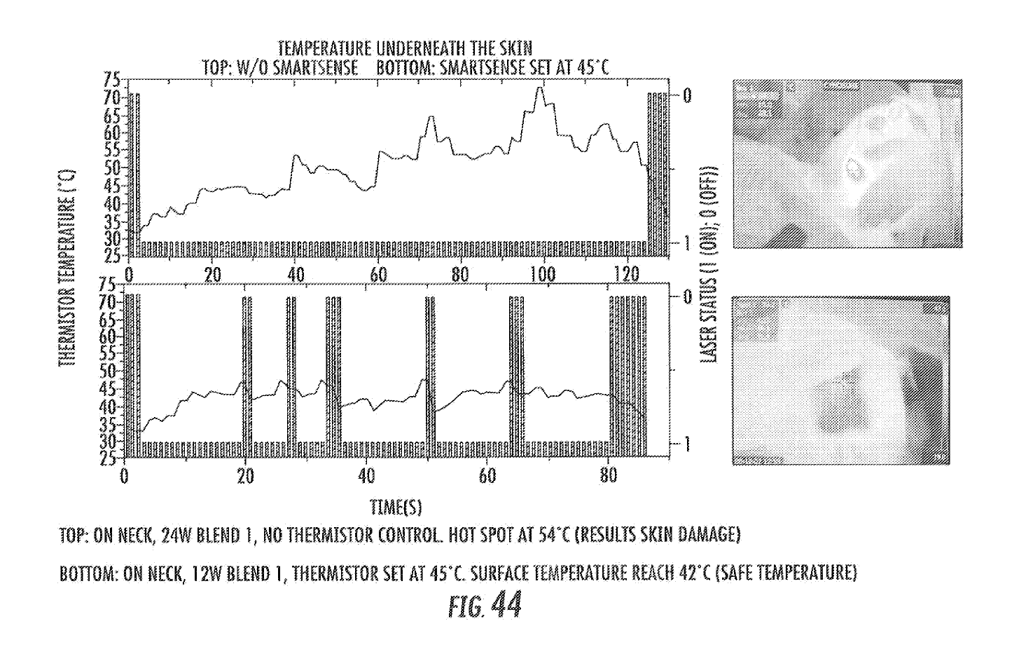

If these high powers are used (39-46W=faster treatments), the possibility exists of excessively high temperatures (>70 C) (FIG. 43). While the body may tolerate some small regions of excessive heat, no physician believes it is desirable. But even with these high power sources capable of undesirable effects, the thermistor can govern the source to achieve tissue temperatures with surprisingly accuracy, ensure safety and optimize the clinical results. An optimized system uses high power to achieve the set point quickly and maintain the set point as efficiently as possible.

FIG. 44 shows two cases where there is no control at high power and where control is applied at low power. One results in localized second-degree burns and a poor distribution of the laser energy subdermally and the other shows a good, even distribution with no chance of burning. These are different doctors with entirely different techniques. The localized burn could have been prevented with the thermistor feedback.

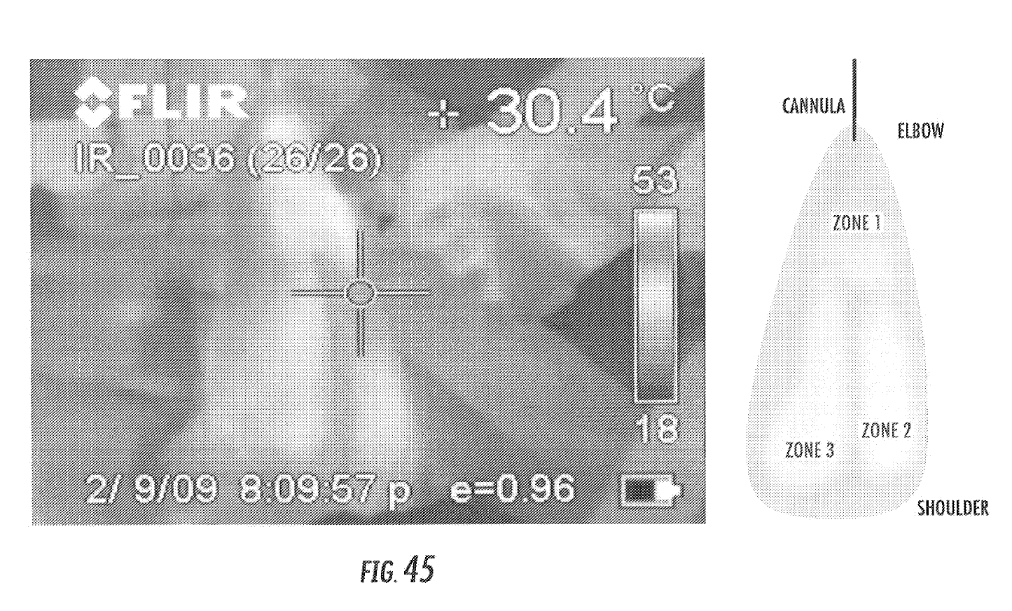

Taking this idea a step further, not only is the safety of even the highest power system kept in check and outcomes yield more consistent results, but also procedures can be designed to reach isothermal set points tuned to the procedural goals. For example, in FIG. 45 the following infrared image indicates the surface temperature of a treatment to an upper arm, three zones shown in yellow-green to have a very even temperature profile. The laser cannula can be shown entering the very top of the picture to the right of the R in FLIR. This image can be matched to the second graph where a set point of 45 C was chosen. All three zones were treated at a set point of 45C. However, due to the capabilities of this system described here, each of the surgical zones could be treated with a different temperature.

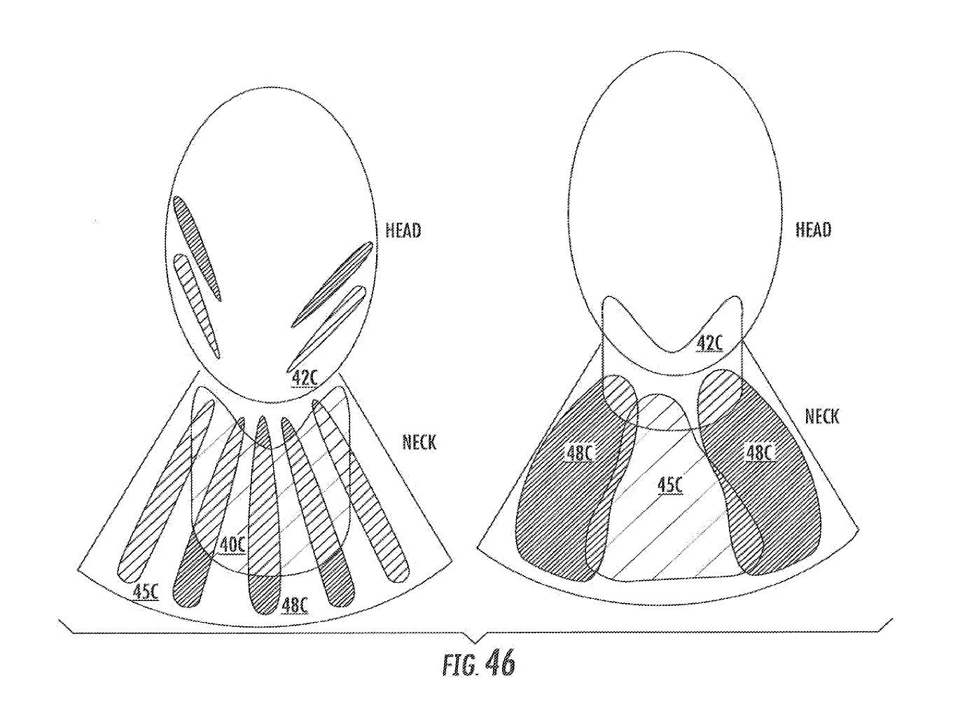

In the case of a hypothetical face and/or neck treatment depicted in FIG. 46, maps could be created as part of the surgical plan with a surgical marker designating the isothermal zones. The tightening effect could be tuned and applied where the tissue anatomically/physiologically/empirically responds the best. It can possibly be feathered by treating some areas with lower temperature near the borders to untreated tissue and higher temperatures at the core of the treatment area. By applying a uniform temperature optimized for tissue shrinkage, tensor paths could be laid down to pull tissue on an intended axis, not just random heating, but biased shrinkage to control results to a new level.

The development of skin therapies where tightening and fat removal are surgical goals will benefit greatly from a system which can precisely heat tissue to a clinical set point. The incorporation of isothermal surgical zones at the onset of the surgical procedure will enable surgeons to optimize outcomes beyond that which is currently available today.

It is often thought that by inhibiting the laser as the cannula is passed through heated tissue, we necessarily slow down the procedure. This is only true if the laser peak uninhibited power is kept constant. By using the temperature control system described herein it is possible and safe to use a higher powered laser to apply the energy. Since a higher powered laser can safely be used with a temperature control system, it follows that procedure times would necessarily decrease. This effect is only limited by the laser inhibition (temperature regulation) as the cannula moves through already heated tissue. In the end, increasing laser power makes the procedure faster, while temperature regulation makes the procedure a small amount slower but allows the use of a higher powered laser. The net result of which, is a faster, more evenly treating laser system.

What is claimed is:

1. A method of treating cellulite in a patient comprising:
inserting an optical delivery device into the patient such that a light emitting portion of the device is located below the interface between the dermis and the hypodermis of the patient;
delivering therapeutic light from the light emitting portion of the delivery device to heat a target region located proximal to the interface to cause thermal damage in the target region without causing substantial thermal damage to dermal and epidermal tissue located above the target region.

2. The method of claim 1, wherein the step of delivering therapeutic light from the light emitting portion of the delivery device to heat a target region located proximal to the interface comprises substantially localizing the heating of the dermis to within a desired distance above the interface.

3. The method of claim 2, wherein the desired distance is about 0.5 mm or less.

4. The method of claim 3, wherein the desired distance is about 1.0 mm or less.

5. The method any preceding claim, comprising heating the target region proximal the interface to a temperature of about 50 C. or more while maintaining the upper dermal and epidermal tissue located above the target region at a temperature of about 42 C or less.

6. The method of any preceding claim, wherein the target region comprises at least one adipocyte extending through the interface into the dermis, and wherein the thermal damage comprises thermal denaturing of the adipocyte.

7. The method of any preceding claim, wherein the target region comprises connective tissue which connects the dermis to underlying hypodermal tissue, and wherein the thermal damage comprises damage to the connective tissue.

8. The method of any preceding claim, further comprising:
inserting a tip of a cannula into the target region;
moving the tip of the cannula within the target region to cause mechanical damage to tissue in the region.

9. The method of claim 8, wherein the target region comprises connective tissue which connects the dermis to underlying hypodermal tissue, and wherein the mechanical damage comprises damage to the connective tissue.

10. The method of claim 8 or 9, wherein the optical delivery device comprises an optical fiber having at least a portion housed in the cannula.

11. The method of any preceding claim, wherein the optical delivery device comprises a side firing optical fiber which extends along a longitudinal axis from a first end to a second end, and wherein the step of delivering therapeutic light from the light emitting portion of the delivery device comprises:
receiving therapeutic light at the first end of the fiber;
transmitting the therapeutic light to the second end of the fiber; and
emitting at a first portion of the therapeutic light from the second end of the fiber along a direction transverse to the longitudinal axis of the fiber.

12. The method of claim 11, wherein the step of delivering therapeutic light from the light emitting portion of the delivery device further comprises emitting a second portion of the therapeutic light from the second end of the fiber along a direction substantially parallel to the longitudinal axis of the fiber.

13. The method of claim 12, further comprising:
directing the first portion of therapeutic light towards the interface; and
directing the second portion of light into the hypodermis.

14. The method of any preceding claim, wherein the therapeutic light comprises laser light.

15. The method of any preceding claim, wherein the therapeutic light comprises light having a wavelength in the visible or near-infrared.

16. The method of any preceding claim, wherein the treatment light has a wavelength of about 1440 nm.

17. The method of any preceding claim, wherein the delivered therapeutic light has a total power in the range of 4 W to 20 W.

18. The method of any preceding claim, wherein the delivered therapeutic light has a total power of about 8 W.

19. The method of any preceding claim, wherein the delivered therapeutic light has a power density in the range of 200 W/cm̂2 to 20,000 W/cm̂2 at the target region.

20. The method of any preceding claim, wherein the step of delivering therapeutic light from the light emitting portion of the delivery device comprises delivering a series of light pulses.

21. The method of claim 21, wherein the series of pulses comprises a pulse having a duration of about 0.5 ms.

22. The method of claim 20 or 21, wherein the series of pulses comprises a pulse having a duration in the range of about 0.1 ms to about 1.0 ms.

23. The method of claim 20, 21 or 22, wherein the series of pulses has a repetition rate of about 40 Hz.

24. The method of claim 20, 21, 22 or 23, wherein the series of pulses has a repetition rate in the range of about 10 to about 100 Hz.

25. The method of any preceding claim, wherein the optical delivery device comprises at least one sensor, and further comprising:
using the at least one sensor, generating a signal indicative of at least one property of the delivery device or the target region;
controlling the delivery of therapeutic light based on the sensor signal.

26. The method of claim 25, wherein the property of the delivery device or the target region comprises at least one selected from the list consisting of: a position of the optical delivery device, a movement of the optical delivery device, temperature of the optical delivery device, a tissue type in the vicinity of the optical delivery device, an amount of energy delivered by the optical delivery device, and a temperature of tissue in the target region.

27. The method of claim 25 or 26, wherein the sensor comprises at least one selected from the list consisting of: a thermister, an accelerometer, and a color sensor.

28. The method of claim 25, 26, or 27 further comprising generating a display based on signal indicative of at least one property of the delivery device or the target region.

29. The method of claim 28, wherein the display comprises a temperature map of a region of the patient undergoing treatment.

30. A an apparatus for treating cellulite in a patient comprising:
an optical delivery device having a light emitting portion configured to be inserted into the patient such that the light emitting portion of the device is located below the interface between the dermis and the hypodermis of the patient;
a controller to control the delivery of therapeutic light from the light emitting portion of the delivery device to heat a target region located proximal to the interface to cause thermal damage in the target region without causing substantial thermal damage to dermal and epidermal tissue located above the target region.

31. A method of treating an area of skin located on or near the face or neck of a patient comprising:
inserting an optical delivery device into the patient such that a light emitting portion of the device is proximal to an interface between the dermis of the skin and the underlying fascia of the patient;
delivering therapeutic light from the light emitting portion of the delivery device to heat a target region located proximal to the interface to cause thermal damage in the target region without causing substantial thermal damage to dermal and epidermal tissue located above the target region.

32. The method of claim 31, wherein the step of delivering therapeutic light from the light emitting portion of the delivery device to heat a target region located proximal to the interface comprises substantially localizing the heating of the dermis to within a desired distance above the interface.

33. The method of claim 32, wherein the desired distance is about 0.5 mm or less.

34. The method of claim 3, wherein the desired distance is about 1.0 mm or less.

35. The method any preceding claim, comprising heating the target region proximal the interface to a temperature of about 50 C. or more while maintaining the upper dermal and epidermal tissue located above the target region at a temperature of about 42 C or less.

36. The method of any preceding claim, wherein the target region extends along the interface, and wherein delivering therapeutic light from the light emitting portion of the delivery device to heat a target region comprises moving the light emitting portion of the optical delivery device along the interface while delivering the therapeutic light.

37. The method of claim 36, further comprising modulating the delivery of therapeutic light while moving the light emitting portion of the optical delivery device along the interface to form localized sub regions of thermal damage within the target region.

38. The method of any preceding claim, further comprising:
inserting a tip of a cannula into the target region;
moving the tip of the cannula within the target region to cause mechanical damage to tissue in the region.

39. The method of claim 38, wherein the target region comprises connective tissue which connects the dermis to underlying fascia, and wherein the mechanical damage comprises damage to the connective tissue.

40. The method of claim 38 or 39, wherein the optical delivery device comprises an optical fiber having at least a portion housed in the cannula.

41. The method of any preceding claim, wherein the optical delivery device comprises a side firing optical fiber which extends along a longitudinal axis from a first end to a second end, and wherein the step of delivering therapeutic light from the light emitting portion of the delivery device comprises:
receiving therapeutic light at the first end of the fiber;
transmitting the therapeutic light to the second end of the fiber; and
emitting at a first portion of the therapeutic light from the second end of the fiber along a direction transverse to the longitudinal axis of the fiber.

42. The method of claim 41, wherein the step of delivering therapeutic light from the light emitting portion of the delivery device further comprises emitting a second portion of the therapeutic light from the second end of the fiber along a direction substantially parallel to the longitudinal axis of the fiber.

43. The method of claim 12, further comprising:
directing the first portion of therapeutic light towards the interface; and
directing the second portion of light into the underlying fascia.

44. The method of any preceding claim, wherein the therapeutic light comprises laser light.

45. The method of any preceding claim, wherein the therapeutic light comprises light having a wavelength in the visible or near-infrared.

46. The method of any preceding claim, wherein the treatment light has a wavelength of about 1440 nm.

47. The method of any preceding claim, wherein the delivered therapeutic light has a total power in the range of 4 W to 20 W.

48. The method of any preceding claim, wherein the delivered therapeutic light has a total power of about 8 W.

49. The method of any preceding claim, wherein the delivered therapeutic light has a power density in the range of 200 W/cm̂2 to 20,000 W/cm̂2 at the target region.

50. The method of any preceding claim, wherein the step of delivering therapeutic light from the light emitting portion of the delivery device comprises delivering a series of light pulses.

51. The method of claim 51, wherein the series of pulses comprises a pulse having a duration of about 0.5 ms.

52. The method of claim 50 or 51, wherein the series of pulses comprises a pulse having a duration in the range of about 0.1 ms to about 1.0 ms.

53. The method of claim 50, 51 or 52, wherein the series of pulses has a repetition rate of about 40 Hz.

54. The method of claim 50, 51, 52 or 53, wherein the series of pulses has a repetition rate in the range of about 10 to about 100 Hz.

55. The method of any preceding claim, wherein the optical delivery device comprises at least one sensor, and further comprising:
using the at least one sensor, generating a signal indicative of at least one property of the delivery device or the target region;
controlling the delivery of therapeutic light based on the sensor signal.

56. The method of claim 55, wherein the property of the delivery device or the target region comprises at least one selected from the list consisting of: a position of the optical delivery device, a movement of the optical delivery device, temperature of the optical delivery device, a tissue type in the vicinity of the optical delivery device, an amount of energy delivered by the optical delivery device, and a temperature of tissue in the target region.

57. The method of claim 55 or 56, wherein the sensor comprises at least one selected from the list consisting of: a thermister, an accelerometer, and a color sensor.

58. The method of claim 55, 56, or 57 further comprising generating a display based on signal indicative of at least one property of the delivery device or the target region.

59. The method of claim 58, wherein the display comprises a temperature map of a region of the patient undergoing treatment.

60. An apparatus for treating an area of skin located on or near the face or neck of a patient comprising:
an optical delivery device having a light emitting portion configured to be inserted into the patient such that a light emitting portion of the device is proximal to an interface between the dermis of the skin and the underlying fascia of the patient;
a controller to control the delivery of therapeutic light from the light emitting portion of the delivery device to heat a target region located proximal to the interface to cause thermal damage in the target region without causing substantial thermal damage to dermal and epidermal tissue located above the target region.

61. The apparatus of claim 60 further comprising a temperature map display.

62. A thermal surgical apparatus comprising:
a handpiece comprising a hollow cannula extending from the handpiece to a distal end, the distal end of the cannula having an outer surface comprising a recess;
an optical fiber extending at least partially along the hollow cannula to the distal end and configured to deliver therapeutic light from a therapeutic light source to a treatment region located proximal the distal end of the cannula;
a temperature sensor located at least partially within the in the recess.

63. The apparatus of claim 62, further comprising a thermally non-conductive inner material layer disposed between the thermister and the outer surface of the cannula.

64. The apparatus of claim 63, wherein the thermally non-conductive material layer substantially thermally insulates the temperature sensor from the outer surface of the cannula.

65. The apparatus of claim 64, wherein the insulating material comprises at least one material from the list consisting of: a plastic, a polymer, polystyrene, and an adhesive material.

66. The apparatus of any preceding claim further comprising an outer material layer disposed on the outer surface of the cannula to secure the temperature sensor within the recess.

67. The apparatus of claim 66, wherein the outer material layer comprises a sleeve disposed about at least a portion of the outer layer of the cannula to secure the temperature sensor within the recess.

68. The apparatus of claim 66 or 67, wherein the outer material layer comprises a thermally conductive material.

69. The apparatus of claim 67, wherein the thermally conductive material comprises at least one material from the list consisting of: a metal, a metal foil, a thermally conductive polymer, a thermally conductive plastic, and a thermally conductive silicone.

70. The apparatus of any of claims 66-69, wherein the outer material layer has higher thermal conductivity than an inner material layer disposed between the thermister and the outer surface of the cannula.

71. The apparatus of any preceding claim, wherein the temperature sensor is a thermister.

72. The apparatus of claim 71 wherein the thermister has a characteristic size of about 1 mm or less.

73. The apparatus of claim 71 or 72, wherein the thermister is characterized by a response time of about 250 ms or less.

74. The apparatus of any preceding claim further comprising a processor in communication with the temperature sensor to receive a signal from the sensor indicative of a temperature in the treatment region and control the delivery of therapeutic light from the therapeutic light source through the optical fiber.

75. The apparatus of claim 74, wherein the handpiece comprises at least one additional sensor configured to in communication with the processor, and wherein:
the additional sensor is configured to generate a signal indicative of at least one property of the handpiece or the treatment region;
the processor is configured to control the delivery of therapeutic light to the treatment region based on the sensor signal.

76. The apparatus of claim 75, wherein the property of the hanpiece or the target region comprises at least one selected from the list consisting of: a position of the handpiece, a movement of the handpiece, a temperature of the handpiece, a tissue type in the vicinity of the distal end of the cannula, an amount of energy delivered to the target region, and a temperature of tissue in the target region.

77. The apparatus of claim 75 or 76, wherein the sensor comprises at least one selected from the list consisting of: a thermister, an inertial sensor, an accelerometer, a gyroscope, and a color sensor.

78. The apparatus of any preceding claim, wherein the distal end of the cannula comprises at least one suction port.

79. The apparatus of any preceding claim, wherein the recess comprises a slot in the cannula.

80. The apparatus of any preceding claim, wherein the substantially the entire temperature sensor is housed within the recess.

81. The apparatus of any preceding claim, wherein at least a portion of the optical fiber is located within the hollow cannula.

82. The apparatus of any preceding claim, wherein the hollow cannula comprises a suction cannula, and further comprising a treatment cannula housing at least a portion of the optical fiber.
